(19) United States
(12) Patent Application Publication
(10) Pub. No.: US 2025/0282838 A1
(43) Pub. Date: Sep. 11, 2025

(54) Multi-Epitope Vaccine for the Treatment of Alzheimer's Disease

(71) Applicant: OTHAIR PROTHENA LIMITED, Dublin (IE)

(72) Inventors: Robin BARBOUR, Walnut Creek, CA (US); Gene KINNEY, Boca Raton, FL (US); Wagner ZAGO, San Carlos, CA (US); Tarlochan S. NIJJAR, Orinda, CA (US)

(21) Appl. No.: 19/064,783

(22) Filed: Feb. 27, 2025

Related U.S. Application Data
(63) Continuation of application No. 18/594,435, filed on Mar. 4, 2024, now Pat. No. 12,247,056.
Application No. 18/594,435 is a division of application No. 18/328,528, filed on Jun. 2, 2023, now Pat. No. 11,945,849.
Application No. 18/328,528 is a division of application No. 17/925,813, filed on Nov. 16, 2022.
(60) Provisional application No. 63/027,150, filed on May 19, 2020.
(60) Provisional application No. 63/062,903, filed on Aug. 7, 2020.
(60) Provisional application No. 63/140,917, filed on Jan. 24, 2021.

(51) Int. Cl. C07K 14/47; A61K 39/00; A61K 39/39; A61P 25/28; A61P 37/04

(52) U.S. Cl. CPC C07K 14/4711; A61K 39/0007; A61K 39/39; A61P 25/28; A61P 37/04; A61K 2039/53; A61K 2039/545; A61K 2039/55516; A61K 2039/55544; A61K 2039/55555; A61K 2039/55572; A61K 2039/55577; A61K 2039/575; A61K 2039/6037; C07K 2319/00

(57) ABSTRACT

The disclosure provides peptide compositions and immunotherapy compositions comprising an amyloid-beta (Aβ) peptide and a tau peptide. The disclosure also provides methods of treating or effecting prophylaxis of Alzheimer's disease or other diseases with beta-amyloid deposition in a subject, including methods of clearing deposits, inhibiting or reducing aggregation of Aβ and/or tau, blocking the uptake by neurons, clearing amyloid, and inhibiting propagation of tau seeds in a subject having or at risk of developing Alzheimer's disease or other diseases containing tau and/or amyloid-beta accumulations. The methods include administering to such patients the compositions comprising an amyloid-beta (Aβ) peptide and a tau peptide.

### RELATED APPLICATIONS

This application is a divisional application of U.S. application Ser. No. 18/594,435, filed Mar. 4, 2024, now U.S. Pat. No. 12,247,056, which is a divisional application of U.S. application Ser. No. 18/328,528, filed Jun. 2, 2023, now U.S. Pat. No. 11,945,849, which is a divisional application of U.S. application Ser. No. 17/925,813, filed Nov. 16, 2022, which is a U.S. national phase application under 35 U.S.C. § 371 of International Application No. PCT/US2021/033222, filed May 19, 2021, and which claims the benefit of U.S. Provisional Patent Application Ser. No. 63/140,917, filed Jan. 24, 2021, U.S. Provisional Patent Application Ser. No. 63/062,903, filed Aug. 7, 2020, and U.S. Provisional Patent Application Ser. No. 63/027,150, filed May 19, 2020, each of which is incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Mar. 14, 2024, having the file name “20-050-WO-US-DIV_SequenceListing.xml” and is 1,171,456 bytes in size.

### FIELD

The disclosure relates to the technical fields of immunology and medicine, and in particular to the treatment of Alzheimer's disease and other diseases of protein misfolding.

### BACKGROUND

Alzheimer's disease (AD) is a progressive disease resulting in senile dementia. Broadly speaking, the disease falls into two categories: late onset, which occurs in old age (65+ years) and early onset, which develops well before the senile period, i.e., between 35 and 60 years. In both types of disease, the pathology is the same, but the abnormalities tend to be more severe and widespread in cases beginning at an earlier age. The disease is characterized by at least two types of lesions in the brain, neurofibrillary tangles and senile plaques. Neurofibrillary tangles are intracellular deposits of microtubule associated tau protein consisting of two filaments twisted about each other in pairs. Senile plaques (i.e., amyloid plaques) are areas of disorganized neuropil up to 150 μm across with extracellular amyloid deposits at the center which are visible by microscopic analysis of sections of brain tissue. The accumulation of amyloid plaques within the central nervous system is also associated with Down's syndrome and other cognitive disorders, Cerebral amyloid angiopathy (CAA), and the ocular disease Age-Related Macular Degeneration.

A principal constituent of the plaques is a peptide termed Aβ or β-amyloid peptide. Aβ peptide is a 4-kDa internal fragment of 38-43 amino acids of a larger transmembrane glycoprotein named amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The cognitive and physical symptoms associated with this type of neural deterioration characterize Alzheimer's disease.

Another protein reported to occur at increased levels in Alzheimer's patients relative to the general population is tau, the principal constituent of neurofibrillary tangles, which together with amyloid plaques are a hallmark characteristic of Alzheimer's disease. Tau tangles constitute abnormal fibrils measuring 10 nm in diameter occurring in pairs wound in a helical fashion with a regular periodicity of 80 nm. The tau within neurofibrillary tangles is abnormally phosphorylated (hyperphosphorylated) with phosphate groups attached to specific sites on the molecule. Severe involvement of neurofibrillary tangles is seen in the layer II neurons of the entorhinal cortex, the CA1 and subicular regions of the hippocampus, the amygdala, and the deeper layers (layers III, V, and superficial VI) of the neocortex in Alzheimer's disease. Tau pathologies are known to correlate to cognitive decline.

Accordingly, there exists the need for new therapies and reagents for the prevention or treatment of Alzheimer's disease, in particular, therapies and reagents capable of causing an immune response to the Aβ and Tau present in patients.

### SUMMARY

In some embodiments, disclosure is directed to a polypeptide including a first peptide comprising 3-10 amino acids from residues 1-10 of SEQ ID NO:01 linked to a second peptide including 3-13 amino acids from residues 244-400 of SEQ ID NO:02. For example, the second peptide may be from the microtubule binding region (MTBR) of tau (residues 244-372 of SEQ ID NO:02). The first peptide may be N-terminal to the second peptide or the first peptide may be C-terminal to the second peptide. In addition, the first peptide may include an amino acid sequence of one of SEQ ID NOS: 3 to 38 or SEQ ID NOS: 1002 to 1057 and the second peptide may include an amino acid sequence of one of SEQ ID NOS: 39-56, 83-86, or 146-996. For example, the first polypeptide may be DAEFRHD (SEQ ID NO:06), DAEFR (SEQ ID NO: 08), or EFRHD (SEQ ID NO:21), and the second polypeptide may be 5-13 amino acids, for example QIVYKPV (SEQ ID NO:39), EIVYKSV (SEQ ID NO:42), EIVYKSP (SEQ ID NO: 43), EIVYKPV (SEQ ID NO:44), NIKHVP (SEQ ID NO:48), VKSKIGST (SEQ ID NO: 801), SKIGSTEN (SEQ ID NO:817), TENLKHQP (SEQ ID NO:695), ENLKHQPG (SEQ ID NO: 689), SKIGSTDNIKH (SEQ ID NO:985), SKIGSKDNIKH (SEQ ID NO:986), or SKIGSLDNIKH (SEQ ID NO:988).

In other embodiments, the first peptide and second peptide may be linked by a cleavable linker, which may be an amino acid sequence. A cleavable peptide linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the cleavable peptide linker is 1 amino acid, 2 amino acids, 3 amino acids, 4 amino acids, 5 amino acids, 6 amino acids, 7 amino acids, 8 amino acids, 9 amino acids, or 10 amino acids. For example, the linker may be arginine-arginine (Arg-Arg), arginine-valine-arginine-arginine (Arg-Val-Arg-Arg (SEQ ID NO:69)), valine-citrulline (Val-Cit), valine-arginine (Val-Arg), valine-lysine (Val-Lys), valine-alanine (Val-Ala), phenylalanine-lysine (Phe-Lys), glycine-alanine-glycine-alanine (Gly-Ala-Gly-Ala; SEQ ID NO:80), Ala-Gly-Ala-Gly (SEQ ID NO:81), or Lys-Gly-Lys-Gly (SEQ ID NO:82). In particular embodiments, the polypeptide may be DAEFRHDRRQIVYKPV (SEQ ID NO:57), DAEFRHDRREIVYKSV (SEQ ID NO:58), DAEFRHDRRVKSKIGSTGGC (SEQ ID NO:997), DAEFRHDRRSKIGSTENGGC (SEQ ID NO:998), DAEFRHDRRTENLKHQPGGC (SEQ ID NO: 999), DAEFRHDRRENLKHQPGGGC (SEQ ID NO:1000), or DAEFRHDRRSKIGSKDNIKHGGC (SEQ ID NO:1001).

In further embodiments, the polypeptide may include a linker to a carrier at a C-terminal portion of the polypeptide, or at a N-terminal portion of the polypeptide. A linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the linker is 1 amino acid, 2 amino acids, 3 amino acids, 4 amino acids, 5 amino acids, 6 amino acids, 7 amino acids, 8 amino acids, 9 amino acids, or 10 amino acids. For example, the linker may include an amino acid sequence of GG, GGG, AA, AAA, KK, KKK, SS and SSS. In addition, the linker to the carrier, if present at the C-terminus, may include a C-terminal cysteine (C). Alternatively, the linker to the carrier, if present at the N-terminus, may include a N-terminal cysteine (C). For example, the polypeptide may include the amino acid sequence of DAEFRHDRRQIVYKPVXXC (SEQ ID NO: 70), wherein XX and C are independently optional and, if present, XX can be GG, AA, KK, SS, GAGA (SEQ ID NO:80), AGAG (SEQ ID NO:81), or KGKG (SEQ ID NO:82). For example, the polypeptide may include the amino acid sequence of DAEFRHDRREIVYKSVXXC (SEQ ID NO:79), wherein XX and C are independently optional and, if present, XX can be GG, AA, KK, SS, GAGA (SEQ ID NO:80), AGAG (SEQ ID NO:81), and KGKG (SEQ ID NO:82).

In other embodiments, the disclosure is directed to an immunotherapy composition including the polypeptides of the disclosure, wherein the polypeptide may be linked to a carrier. The carrier may include serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

Still further, embodiments of the disclosure are directed to a pharmaceutical formulation includes the polypeptides or the immunotherapy compositions of the disclosure, and including at least one adjuvant. The adjuvant may be aluminum hydroxide, aluminum phosphate, aluminum sulfate, 3 De-O-acylated monophosphoryl lipid A (MPL), QS-21, QS-18, QS-17, QS-7, TQL1055, Complete Freund's Adjuvant (CFA), Incomplete Freund's Adjuvant (IFA), oil in water emulsions (such as squalene or peanut oil), CpG, polyglutamic acid, polylysine, Adda Vax™, MF59®, and combinations thereof. In addition, the formulation may include a liposomal formulation, a diluent, or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Still further, embodiments of the disclosure are directed to an immunotherapy composition including a first peptide sequence comprising 3-10 amino acid residues from the first ten N-terminal residues of SEQ ID NO:01 and a second peptide sequence comprising 3-13 amino acids from residues 244-400 of SEQ ID NO:02. The first peptide may include an amino acid sequence of one of SEQ ID NOS: 3 to 38 or SEQ ID NOS: 1002 to 1057, and the second peptide may include an amino acid sequence of one of SEQ ID NOS: 39 to 56, SEQ ID NOS: 83-86 or SEQ ID NOS: 146-996. Each of the first peptide and the second peptide may include a linker to a carrier at a C-terminal portion of the polypeptide, or at a N-terminal portion of the polypeptide. When present, the linker may include an amino acid sequence selected from GG, GGG, AA, AAA, KK, KKK, SS, SSS, GAGA (SEQ ID NO:80), AGAG (SEQ ID NO:81), and KGKG (SEQ ID NO:82), and may include a C-terminal cysteine (C). In some embodiments, where the C-terminal residues in the immunogen are either IVYKPV (SEQ ID NO:194), VYKPV (SEQ ID NO:195), YKPV (SEQ ID NO:196), KPV, or PV the linker is an amino acid linker that does not have a N-terminal glycine (e.g., GG, GAGA (SEQ ID NO:80)). The carrier may include serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

In addition, the immunotherapy composition may include at least one pharmaceutically acceptable diluent and/or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

The immunotherapy composition may be included in a pharmaceutical composition including the immunotherapy composition and at least one adjuvant such as aluminum hydroxide, aluminum phosphate, aluminum sulfate, 3 De-O-acylated monophosphoryl lipid A (MPL), QS-21, QS-18, QS-17, QS-7, TQL1055, Complete Freund's Adjuvant (CFA), Incomplete Freund's Adjuvant (IFA), oil in water emulsions (such as squalene or peanut oil), CpG, polyglutamic acid, polylysine, Adda Vax™, MF59®, and combinations thereof.

Embodiments of the disclosure are also directed to nucleic acid sequences encoding the polypeptides and the immunotherapy compositions of the disclosure. The nucleic acids may be included in a nucleic acid immunotherapy composition including the nucleic acid and at least one adjuvant.

Still further, embodiments of the disclosure are directed to a methods for treating or effecting prophylaxis of Alzheimer's disease in a subject, and methods for inhibiting or reducing aggregation of at least one of Aβ and tau in a subject having or at risk of developing Alzheimer's disease. The methods include administrating to the subject an immunotherapy composition, a nucleic acids immunotherapy composition, or a pharmaceutical formulation of the disclosure.

The methods of the disclosure may include repeating the administering at least a second time, at least a third time, at least a fourth time, at least a fifth time, or at least a sixth time, and may include repeating the administering at an interval of about 21 to about 28 days.

Still further, methods of the disclosure are directed to inducing an immune response in an animal. The methods include administering to the animal a polypeptide, an immunotherapy composition, a pharmaceutical formulation or a nucleic acid immunotherapy composition of the disclosure in a regimen effective to generate an immune response including antibodies that specifically bind to Aβ, tau, or both Aβ and tau. The immune response may include antibodies that specifically bind to the N-terminal region of Aβ and/or the microtubule region of tau.

In other embodiments, the disclosure is directed to an immunization kit including an immunotherapy composition of the disclosure and may include an adjuvant, wherein the immunotherapy composition may be in a first container and the adjuvant may be a second container.

Still further, the disclosure is directed to a kit including a nucleic acid immunotherapy composition of the disclosure and may include an adjuvant. The nucleic acid may be in a first container and the adjuvant may be in a second container.

### BRIEF DESCRIPTION OF THE FIGURES

FIG. 1 shows the results of an experiment comparing the geometric mean titers of Guinea Pig serum for immunogen DAEFRHDRRQIVYKPV (SEQ ID NO:57) on monomeric Aβ amino acids 1-28 (DAEFRHDSGYEVHHQKLFFAEDVGSNKG; SEQ ID NO:67), soluble aggregate species of Aβ (Aβ 42), full length tau, and tau MTBR epitope containing GGGSVQIVYKPVDLS (SEQ ID NO:68) containing an N-terminal biotin.

FIG. 2A shows the results of an experiment comparing the titers of Guinea pig serum for a single peptide immunogen versus dual peptide immunogens of the disclosure on all forms of Aβ and full-length tau protein (DAEFRHD is SEQ ID NO:6; QIVYKPV is SEQ ID NO: 39; DAEFRHDRRQIVYKPV is SEQ ID NO:57).

FIG. 2B shows the results of an experiment comparing the titers of Guinea pig serum for a single peptide immunogen versus dual peptide immunogens of the disclosure on all forms of Aβ and full-length tau protein (DAEFRHD is SEQ ID NO:6; QIVYKPV is SEQ ID NO: 39; EIVYKSP is SEQ ID NO:43; DAEFRHDRRQIVYKPV is SEQ ID NO:57).

FIG. 3A shows staining of Aβ and Tau pathology in fresh frozen human AD brain tissue using serum (1:300 dilution) from a Guinea pig vaccinated with Immunogen 9

(DAEFRHDRRQIVYKPVGGC; SEQ ID NO: 59).

FIG. 3B shows staining of Aβ and Tau pathology in fresh frozen human AD brain tissue using serum (1:300 dilution) from a Guinea pig vaccinated with Immunogen 9

(DAEFRHDRRQIVYKPVGGC; SEQ ID NO: 59).

FIG. 3C shows staining of Aβ and Tau pathology in fresh frozen human AD brain tissue using serum (1:1500 dilution) from a Guinea pig vaccinated with Immunogen 9

(DAEFRHDRRQIVYKPVGGC; SEQ ID NO: 59).

FIG. 3D shows lack of staining of Aβ and Tau pathology in fresh frozen human AD brain tissue using serum (1:300 dilution) from an unvaccinated Guinea pig.

FIG. 3E shows lack of staining of Aβ and Tau pathology in fresh frozen human AD brain tissue using serum (1:300 dilution) from an unvaccinated Guinea pig.

FIG. 3F shows lack of staining of Aβ and Tau pathology in fresh frozen human AD brain tissue using serum (1:1500 dilution) from an unvaccinated Guinea pig.

FIG. 4A shows that Guinea pig serum from animals vaccinated with a dual immunogen peptide, DAEFRHDRRQIVYKPVGGC (SEQ ID NO:59; Immunogen 9), inhibited Abeta soluble aggregates binding to primary neurons in a dose-dependent manner.

FIG. 4B shows examples of Ab staining on primary neurons in the presence or absence of Guinea pig serum from animals vaccinated with a dual immunogen peptide,

(SEQ ID NO: 59; Immunogen 9)
DAEFRHDRRQIVYKPVGGC.

FIG. 5A shows that mice vaccinated with dual peptide antigens with various linkers produce similar titers to Aβ and Tau.

FIG. 5B shows that mice vaccinated with dual peptide antigens with the Tau sequence EIVYKSP (SEQ ID NO:43) produce titers to Aβ and Tau.

FIG. 6A shows staining of Aβ and Tau pathology in fresh frozen human AD brain tissue using serum (1:1000 dilution) from a mouse vaccinated with Immunogen 19.

FIG. 6B shows staining of Aβ and Tau pathology in fresh frozen human AD brain tissue using serum (1:1000 dilution) from a mouse vaccinated with Immunogen 19.

FIG. 6C shows lack of staining of Aβ and Tau pathology in fresh frozen human AD brain tissue using serum (1:1000 dilution) from an unvaccinated mouse.

FIG. 6D shows staining of Tau pathology in fresh frozen human AD brain tissue using anti-Tau antibody m6H3 (0.1 μg/ml).

FIG. 6E shows comparable staining of Tau pathology in fresh frozen human AD brain tissue using serum (1:1500 dilution) from a Guinea pig vaccinated with Immunogen 9

(DAEFRHDRRQIVYKPVGGC; SEQ ID NO: 59).

FIGS. 7A and 7B show titers of dual Aβ-tau immunogen in two injection paradigms. Titers to Aβ1-28, full-length tau and the carrier CRM protein of the two injection schedules ((FIG. 7A) showing 0, 4, 12 and 24 weeks; (FIG. 7B) showing 0, 8 and 24 weeks) over the experiment are expressed as mean titer+/−SEM.

FIG. 8A shows individual animal titers to Aβ and tau two weeks post each injection for the injection schedule of FIG. 7A, i.e., at weeks 2, 6, 14 and 26.

FIG. 8B shows individual animal titers to Aβ and tau two weeks post each injection for the injection schedule of FIG. 7B, i.e., at weeks 2, 14 and 26.

FIG. 9A shows staining of Aβ and Tau pathology in fresh frozen human AD brain tissue using week 26 serum (1:300 dilution) from cynomolgus monkey #1001 vaccinated with Immunogen 15 (DAEFRHDRRQIVYKPVGGC; SEQ ID NO:59) on a four injection vaccination schedule.

FIG. 9B shows a zoomed view of the rectangle indicating staining of Aβ pathology in FIG. 9A.

FIG. 9C shows a shows a zoomed view of the rectangle indicating staining of Tau pathology in FIG. 9A.

FIG. 10A shows staining of Aβ pathology in fresh frozen human AD brain tissue using serum (1:300 dilution) from cynomolgus monkey #1003 vaccinated with Immunogen 15

(DAEFRHDRRQIVYKPVGGC; SEQ ID NO: 59).

FIG. 10B shows modest staining of Tau pathology in fresh frozen human AD brain tissue using serum (1:300 dilution) from cynomolgus monkey #1003.

FIG. 11A shows staining of Aβ pathology in fresh frozen human AD brain tissue using serum (1:300 dilution) from cynomolgus monkey #1501 vaccinated with Immunogen 15

(DAEFRHDRRQIVYKPVGGC; SEQ ID NO: 59).

FIG. 11B shows minimal staining of Tau pathology in fresh frozen human AD brain tissue using serum (1:300 dilution) from cynomolgus monkey #1501.

FIG. 12A shows staining of Aβ pathology in fresh frozen human AD brain tissue using serum (1:300 dilution) from cynomolgus monkey #2501 vaccinated with Immunogen 15

(DAEFRHDRRQIVYKPVGGC; SEQ ID NO: 59).

FIG. 12B shows a lack of staining of Tau pathology in fresh frozen human AD brain tissue using serum (1:300 dilution) from cynomolgus monkey #2501.

FIG. 13 shows animal titers to Aβ and tau 9 weeks post injection at day 0, at 4 weeks and at 8 weeks for the dual Abeta-tau constructs DAEFRHDRRVKSKIGSTGGC (SEQ ID NO: 997); DAEFRHDRRSKIGSTENGGC (SEQ ID NO:998); DAEFRHDRRTENLKHQPGGC (SEQ ID NO:999); DAEFRHDRRENLKHQPGGGC (SEQ ID NO: 1000); and DAEFRHDRRSKIGSKDNIKHGGC (SEQ ID NO:1001).

FIG. 14 shows blocking of tau binding to Heparin by sera from Aβ-tau constructs

(SEQ ID NO: 997)
DAEFRHDRRVKSKIGSTGGC;
(SEQ ID NO: 998)
DAEFRHDRRSKIGSTENGGC;
(SEQ ID NO: 999)
DAEFRHDRRTENLKHQPGGC;
(SEQ ID NO: 1000)
DAEFRHDRRENLKHQPGGGC;
and
(SEQ ID NO: 1001)
DAEFRHDRRSKIGSKDNIKHGGC

FIG. 15A shows competition of tau by sera from DAEFRHDRRVKSKIGSTGGC construct (SEQ ID NO:997).

FIG. 15B shows competition of tau by sera from DAEFRHDRRSKIGSTENGGC construct (SEQ ID NO:998).

FIG. 15C shows competition of tau by sera from

(SEQ ID NO: 999)
DAEFRHDRRTENLKHQPGGC.
construct

FIG. 15D shows competition of tau by sera from

(SEQ ID NO: 1000)
DAEFRHDRRENLKHQPGGGC.
construct

FIG. 15E shows competition of tau by sera from

(SEQ ID NO: 1001)
DAEFRHDRRSKIGSKDNIKHGGC.
construct

FIG. 15F shows competition of tau by sera from controls.

FIG. 15G shows competition of tau by sera from

(SEQ ID NO: 1001)
DAEFRHDRRSKIGSKDNIKHGGC.
construct

FIG. 16A shows Abeta/Tau Dual Vaccine Mouse Serum 2.3_031521 (1:300) Example of staining with DAEFRHDRRVKSKIGSTGGC (SEQ ID NO:997).

FIG. 16B shows Abeta/Tau Dual Vaccine Mouse Serum 3.1_031521 (1:300) Example of staining with DAEFRHDRRSKIGSTENGGC (SEQ ID NO:998).

FIG. 16C shows Abeta/Tau Dual Vaccine Mouse Serum 8.4_031521 (1:300) Example of staining with DAEFRHDRRTENLKHQPGGC (SEQ ID NO:999).

FIG. 16D shows Abeta/Tau Dual Vaccine Mouse Serum 9.2_031521 (1:300) Example of staining with DAEFRHDRRENLKHQPGGGC (SEQ ID NO:1000).

FIG. 16E shows Abeta/Tau Dual Vaccine Mouse Serum 11.1_031521 (1:300) Example of staining with DAEFRHDRRSKIGSKDNIKHGGC (SEQ ID NO: 1001).

### DESCRIPTION

The disclosure provides peptide compositions and immunotherapy compositions comprising an amyloid-beta (Aβ) peptide and a tau peptide. The disclosure also provides methods of treating or effecting prophylaxis of Alzheimer's disease or other diseases with beta-amyloid deposition in a subject, including methods of clearing and preventing formation of deposits, inhibiting or reducing aggregation of Aβ and/or tau, blocking the binding and/or uptake of Aβ and/or tau by neurons, inhibiting transmission of tau species between cells, and inhibiting propagation of pathology between brain regions in a subject having or at risk of developing Alzheimer's disease or other diseases containing tau and/or amyloid-beta accumulations. The methods include administering to such patients the compositions comprising an amyloid-beta (Aβ) peptide and a tau peptide.

A number of terms are defined below. As used herein, the singular forms “a,” “an”, and “the” include plural referents unless the context clearly dictates otherwise. For example, the term “a compound” or “at least one compound” can include a plurality of compounds, including mixtures thereof.

Unless otherwise apparent from the context, the term “about” encompasses insubstantial variations, such as values within a standard margin of error of measurement (e.g., SEM) of a stated value. For example the term “about” as used herein when referring to a measurable value such as a parameter, an amount, a temporal duration, can encompass variations of +/−10% or less, +/−5% or less, or +/−1% or less or less of and from the specified value. Designation of a range of values includes all integers within or defining the range, and all subranges defined by integers within the range. As used herein, statistical significance means p≤0.05.

Compositions or methods “comprising” or “including” one or more recited elements may include other elements not specifically recited. For example, a composition that “comprises” or “includes” a polypeptide sequence may contain the sequence alone or in combination with other sequences or ingredients.

An individual is at increased risk of a disease if the subject has at least one known risk-factor (e.g., age, genetic, biochemical, family history, and situational exposure) placing individuals with that risk factor at a statistically significant greater risk of developing the disease than individuals without the risk factor.

The term “patient” includes human and other mammalian subjects that receive either prophylactic or therapeutic treatment, including treatment naïve subjects. As used herein, the terms “subject” or “patient” refer to any single subject for which treatment is desired, including other mammalian subjects such as, humans, cattle, dogs, guinea pigs, rabbits, and so on. Also intended to be included as a subject are any subjects involved in clinical research trials not showing any clinical sign of disease, or subjects involved in epidemiological studies, or subjects used as controls.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, such as altered gait, as perceived by the subject. A “sign” refers to objective evidence of a disease as observed by a physician.

As used herein, the terms “treat” and “treatment” refer to the alleviation or amelioration of one or more symptoms or effects associated with the disease, prevention, inhibition or delay of the onset of one or more symptoms or effects of the disease, lessening of the severity or frequency of one or more symptoms or effects of the disease, and/or increasing or trending toward desired outcomes as described herein.

The terms “prevention”, “prevent”, or “preventing” as used herein refer to contacting (for example, administering) the peptide(s) or immunotherapy compositions of the present disclosure with a subject before the onset of a disease, with or without Aβ and/or tau pathology already present (primary and secondary prevention), thereby delaying the onset of clinical symptoms and/or alleviating symptoms of the disease after the onset of the disease, compared to when the subject is not contacted with the peptide or immunotherapy compositions, and does not refer to completely suppressing the onset of the disease. In some cases, prevention may occur for limited time after administration of the peptide or immunotherapy compositions of the present disclosure. In other cases, prevention may occur for the duration of a treatment regimen comprising administering the peptide or immunotherapy compositions of the present disclosure.

The terms “reduction”, “reduce”, or “reducing” as used herein refer to decreasing the amount of Aβ and/or tau present in a subject or in tissue of the subject, or suppressing an increase in the amount of Aβ and/or tau present in a subject or in tissue of the subject, which encompasses decreasing or suppressing an increase in (e.g., decreasing the rate of increase) the amount of Aβ and/or tau present, accumulated, aggregated, or deposited in the subject or tissue in the subject. In certain embodiments, the decrease in or suppression of an increase in (e.g., decreasing the rate of increase) the amount of Aβ and/or tau present, accumulated, aggregated, or deposited in the subject refers to an amount of Aβ and/or tau present, accumulated, aggregated, or deposited in the central nervous system (CNS) of the subject. In certain embodiments, the decrease in or suppression of an increase in (e.g., decreasing the rate of increase) the amount of Aβ and/or tau present, accumulated, aggregated, or deposited in the subject refers to an amount of Aβ and/or tau present, accumulated, aggregated, or deposited in the periphery (e.g., peripheral circulatory system) of the subject. In certain embodiments, the decrease in or suppression of an increase in (e.g., decreasing the rate of increase) the amount of Aβ and/or tau present, accumulated, aggregated, or deposited in the subject refers to an amount of Aβ and/or tau present, accumulated, aggregated, or deposited in the brain of the subject. In some embodiments, the Aβ and/or tau reduced is the pathological form(s) of the Aβ (e.g., extracellular plaque deposits of the β-amyloid peptide (Aβ), neuritic amyloid plaques), and/or tau (e.g., neurofibrillary tangles of tau, dystrophic neurites). In yet other embodiments, pathological indicators of neurodegenerative disease are decreased.

The terms “epitope” or “antigenic determinant” refers to a site on an antigen to which B and/or T cells respond, or to a site on an antigen to which an antibody binds. Epitopes can be formed both from contiguous amino acids or from noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, at least 4, at least 5, at least 6, at least 7, at least 8, at least 9, at least 10, at least 11, at least 12, or at least 13 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996).

An “immunogenic agent” or “immunogen” or “antigen” is capable of inducing an immunological response against itself or modified/processed versions of itself upon administration to an animal, optionally in conjunction with an adjuvant. The terms “immunogenic agent” or “immunogen” or “antigen” refer to a compound or composition comprising a peptide, polypeptide or protein which is “antigenic” or “immunogenic” when administered in an appropriate amount (an “immunogenically effective amount”), i.e., capable of inducing, eliciting, augmenting or boosting a cellular and/or humoral immune response and of being recognized by the products of that response (T cells, antibodies). An immunogen can be a peptide, or a combination of two or more same or different peptides, that includes at least 3, at least 4, at least 5, at least 6, at least 7, at least 8, at least 9, at least 10, at least 11, at least 12, or at least 13 amino acids in a liner or spatial conformation.

An immunogen may be effective when given alone or in combination, or linked to, or fused to, another substance (which can be administered at one time or over several intervals). An immunogenic agent or immunogen may include an antigenic peptide or polypeptide that is linked to a carrier as described herein.

A nucleic acid such as DNA or RNA that encodes an antigenic peptide, or polypeptide is referred to as a “DNA [or RNA] immunogen,” as the encoded peptide or polypeptide is expressed in vivo after administration of the DNA or RNA. The peptide or polypeptide can be recombinantly expressed from a vaccine vector, which can be naked DNA or RNA that comprises the peptide or polypeptide coding sequence operably linked to a promoter, e.g., an expression vector or cassette as described herein.

The term “adjuvant” refers to a compound that, when administered in conjunction with an antigen, augments the immune response to the antigen, but when administered alone does not generate an immune response to the antigen. Adjuvants can augment an immune response by several mechanisms including lymphocyte recruitment, stimulation of B and/or T cells, and stimulation of macrophages. An adjuvant may be a natural compound, a modified version of or derivative of a natural compound, or a synthetic compound.

The terms “peptide” and “polypeptide” are used interchangeably herein and refer to a chain of two or more consecutive amino acids. If and when a distinction is made, context makes the meaning clear. For example, if two or more peptides described herein are joined to make a dimeric or multimeric peptide, polypeptide may be used to indicate “poly” or “more than one” peptide.

The term “pharmaceutically acceptable” means that the carrier, diluent, excipient, adjuvant, or auxiliary is compatible with the other ingredients of a pharmaceutical formulation and not substantially deleterious to the recipient thereof.

The terms “immunotherapy” or “immune response” refer to the development of a beneficial humoral (antibody mediated) and/or a cellular (mediated by antigen-specific T cells or their secretion products) response directed against an Aβ and/or tau peptide in a recipient. Such a response can be an active response induced by administration of immunogen (e.g. an Aβ and/or tau peptide). A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

### Amyloid Beta (Aβ)

Aβ (also referred to herein as beta amyloid peptide or Abeta) peptide is about a 4-kDa internal fragment of 38-43 amino acids of APP (Aβ39, Aβ40, Aβ41, Aβ42, and Aβ43). Aβ40, for example, consists of residues 672-711 of APP and Aβ42 consists of residues 673-713 of APP. As a result of proteolytic processing of APP by different secretase enzymes in vivo or in situ, Aβ is found in both a “short form”, 40 amino acids in length, and a “long form”, ranging from 42-43 amino acids in length. Epitopes or antigenic determinants, as described herein, are located within the N-terminus of the Aβ peptide and include residues within amino acids 1-10 and 12-25 of Aβ, for example from residues 1-3, 1-4, 1-5, 1-6, 1-7, or 3-7, 2-4, 2-5, 2-6, 2-7, or 2-8 of Aβ, residues 3-5, 3-6, 3-7, 3-8, or 3-9 of Aβ, or residues 4-7, 4-8, 4-9, or 4-10 residues 12-24, 12-23, 12-22, 13-25, 13-24, 13-23, 13-22, 14-25, 14-24, 14-23, 14-22, 15-25, 15-24, 15-23, or 15-22 of Aβ. For example, from residues 12-17, 12-18, 12-19, 12-20, 12-21, 13-17, 13-18, 13-19, 13-20, 13-21, 13-22, 14-17, 14-18, 14-19, 14-20, 14-21, 14-22, 14-23, 15-17, 15-18, 15-19, 15-20, 15-21, 15-22, 15-23, or 15-24 of Aβ42. Additional examples of epitopes or antigenic determinants include residues 16-18, 16-19, 16-20, 16-21, 16-22, 16-23, 16-24, 16-25, 17-19, 17-20, 17-21, 17-22, 17-23, 17-24 or 17-25 of Aβ42. Other examples of epitopes or antigenic determinants include residues 18-20, 18-21, 18-22, 18-23, 18-24, 18-25, 19-21, 19-22, 19-23, 19-24, 19-25, 20-22, 20-23, 20-24, 20-25, 21-23, 21-24 or 21-25 of Aβ42.

Aβ (Abeta) is the principal component of characteristic plaques of Alzheimer's disease. Aβ is generated by processing of a larger protein APP by two enzymes, termed beta and gamma secretases. Known mutations in APP associated with Alzheimer's disease occur proximate to the site of beta or gamma secretase, or within Aβ. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The physical symptoms associated with this type of neural deterioration characterize Alzheimer's disease.

### Tau

Tau is a protein with a molecular weight of about 50,000 that is normally present in nerve axons or the like, and contributes to microtubular stability. The tau proteins (or t proteins) are a group of six highly-soluble protein isoforms produced by alternative splicing from the gene MAPT (microtubule-associated protein tau). They have roles primarily in maintaining the stability of microtubules in axons and are abundant in the neurons of the central nervous system (CNS). They are less common elsewhere but are also expressed at very low levels in CNS astrocytes and oligodendrocytes. Pathologies and dementias of the nervous system such as Alzheimer's disease and Parkinson's disease are associated with tau proteins that have become hyperphosphorylated insoluble aggregates called neurofibrillary tangles. Pathogenic tau species causes toxic effects through direct binding to cells and/or accumulation inside cells and/or initiation of misfolding processes (seeding) and is can be propagated from one cell to another via cell-to-cell transmission. Toxicity could also happen by neurofibrillary tangles (NFTs), which leads to cell death and cognitive decline. Other tauopathies include, for example, progressive supranuclear palsy, corticobasal syndrome, some frontotemporal dementias, and chronic traumatic encephalopathy.

### Aβ/Tau Polypeptides of an Immunogen

An agent used for active immunization can induce in a patient an immune response and can serve as an immunotherapy. Agents used for active immunization can be, for example, the same types of immunogens used for generating monoclonal antibodies in laboratory animals, and may include 3, 4, 5, 6, 7, 8, 9, 10, 11, 12, or 13 or more contiguous amino acids from a region of Aβ and/or tau peptide. In each of the embodiments of the peptides described herein, the peptides may comprise, consist, or consist essentially of the recited sequences.

In some embodiments of the disclosure, an Aβ/tau immunogen can include an Aβ peptide comprising 3-10 amino acids from residues 1-10 or 12-25 of the N-terminal sequence of Aβ (SEQ ID NO:01) linked to a tau peptide comprising 3-10 amino acids from residues 244-400 of the long form of tau (SEQ ID NO:02). For example, the tau peptide may comprise 3-13 amino acids from the microtubule binding region of tau (residues 344-372 of SEQ ID NO:02).

In some embodiments of the disclosure, the Aβ peptide can include 3-10 amino acids from residues 1-10 or 12-25 of DAEFRHDSGYEVHHQKLVFFAEDVGSNKGAIIGLMVGGVVIA (SEQ ID NO:01). For example, the Aβ peptide is selected from the following:

(SEQ ID NO: 03)

DAEFRHDSGY,

(SEQ ID NO: 04)

DAEFRHDSG,
(SEQ ID NO: 05)
DAEFRHDS,
(SEQ ID NO: 06)
DAEFRHD,
(SEQ ID NO: 07)
DAEFRH,
(SEQ ID NO: 08)
DAEFR,
(SEQ ID NO: 09)
DAEF,
DAE,
(SEQ ID NO: 11)
AEFRHDSGY,
(SEQ ID NO: 12)
AEFRHDSG,
(SEQ ID NO: 13)
AEFRHDS,
(SEQ ID NO: 14)
AEFRHD,
(SEQ ID NO: 15)
AEFRH,
(SEQ ID NO: 16)
AEFR,
AEF,
(SEQ ID NO: 18)
EFRHDSGY,
(SEQ ID NO: 19)
EFRHDSG,
(SEQ ID NO: 20)
EFRHDS,
(SEQ ID NO: 21)
EFRHD,
(SEQ ID NO: 22)
EFRH,
EFR,
(SEQ ID NO: 24)
FRHDSGY,
(SEQ ID NO: 25)
FRHDSG,
(SEQ ID NO: 26)
FRHDS,
(SEQ ID NO: 27)
FRHD,
FRH,
(SEQ ID NO: 29)
RHDSGY,
(SEQ ID NO: 30)
RHDSG,
(SEQ ID NO: 31)
RHDS,
RHD,
(SEQ ID NO: 33)
HDSGY,
(SEQ ID NO: 34)
HDSG,
HDS,
(SEQ ID NO: 36)
DSGY,
DSG,
SGY,
(SEQ ID NO: 1002)
VHHQKLVFFA,
(SEQ ID NO: 1003)
VHHQKLVFF,
(SEQ ID NO: 1004)
VHHQKLVF,
(SEQ ID NO: 1005)
VHHQKLV,
(SEQ ID NO: 1006)
VHHQKL,
(SEQ ID NO: 1007)
HHQKLVFFAE,
(SEQ ID NO: 1008)
HHQKLVFFA,
(SEQ ID NO: 1009)
HHQKLVFF,
(SEQ ID NO: 1010)
HHQKLVF,
(SEQ ID NO: 1011)
HHQKLV,
(SEQ ID NO: 1012)
HHQKL,
(SEQ ID NO: 1013)
HQKLVFFAED,
(SEQ ID NO: 1014)
HQKLVFFAE,
(SEQ ID NO: 1015)
HQKLVFFA,
(SEQ ID NO: 1016)
HQKLVFF,
(SEQ ID NO: 1017)
HQKLVF,
(SEQ ID NO: 1018)
HQKLV,
(SEQ ID NO: 1019)
HQKL,
(SEQ ID NO: 1020)
QKLVFFAEDV,
(SEQ ID NO: 1021)
QKLVFFAED,
(SEQ ID NO: 1022)
QKLVFFAE,
(SEQ ID NO: 1023)
QKLVFFA,
(SEQ ID NO: 1024)
QKLVFF,
(SEQ ID NO: 1025)
QKLVF,
(SEQ ID NO: 1026)
QKLV,
QKL,
(SEQ ID NO: 1028)
KLVFFAEDVG,
(SEQ ID NO: 1029)
KLVFFAEDV,
(SEQ ID NO: 1030)
KLVFFAED,
(SEQ ID NO: 1031)
KLVFFAE,
(SEQ ID NO: 1032)
KLVFFA,
(SEQ ID NO: 1033)
KLVFF,
(SEQ ID NO: 1034)
KLVF,
KLV,
(SEQ ID NO: 1036)
LVFFAEDVG,
(SEQ ID NO: 1037)
LVFFAEDV,
(SEQ ID NO: 1038)
LVFFAED,
(SEQ ID NO: 1039)
LVFFAE,
(SEQ ID NO: 1040)
LVFFA,
(SEQ ID NO: 1041)
LVFF,
LVF,
(SEQ ID NO: 1043)
VFFAEDVG,
(SEQ ID NO: 1044)
VFFAEDV,
(SEQ ID NO: 1045)
VFFAED,
(SEQ ID NO: 1046)
VFFAE,
(SEQ ID NO: 1047)
VFFA,
VFF,
(SEQ ID NO: 1049)
FFAEDVG,
(SEQ ID NO: 1050)
FFAEDV,
(SEQ ID NO: 1051)
FFAED,
(SEQ ID NO: 1052)
FFAE,
FFA,
(SEQ ID NO: 1054)
FAEDVG,
(SEQ ID NO: 1055)
FAEDV,
(SEQ ID NO: 1056)
FAED,
and
FAE.

In certain embodiments, the Aβ peptide is DAEFRHD (SEQ ID NO:06), DAEFR (SEQ ID NO:08) or EFRHD (SEQ ID NO:21).

The tau peptide can correspond to a peptide comprising 3-13 amino acids from residues 244-400 of SEQ ID NO:02. In some embodiments, the fragment is unphosphorylated. In some embodiments, the fragment is phosphorylated. In some embodiments, the tau peptide comprises an amino acid sequence represented by the consensus motif (Q/E)IVYK(S/P) (SEQ ID NO: 996). In some embodiments, the tau peptide comprises an amino acid sequence represented by the consensus motif KXXSXXNX(K/H)H (SEQ ID NO: 995) where X is any amino acid. In some embodiments, the tau peptide is selected from SEQ ID NOS: 146-996. In some embodiments, the tau peptide is selected from the following:

(SEQ ID NO: 39)
QIVYKPV,
(SEQ ID NO: 40)
QIVYKP,
(SEQ ID NO: 41)
QIVYKSV,
(SEQ ID NO: 42)
EIVYKSV,
(SEQ ID NO: 83)
QIVYKS,
(SEQ ID NO: 43)
EIVYKSP,
(SEQ ID NO: 84)
EIVYKS,
(SEQ ID NO: 44)
EIVYKPV,
(SEQ ID NO: 85)
EIVYKP,
(SEQ ID NO: 45)
IVYKSPV,
(SEQ ID NO: 46)
IVYK,
(SEQ ID NO: 86)
CNIKHVPG,
(SEQ ID NO: 47)
CNIKHVP,
(SEQ ID NO: 48)
NIKHVP,
(SEQ ID NO: 49)
HVPGGG,
(SEQ ID NO: 50)
HVPGG,
(SEQ ID NO: 51)
HKPGGG,
(SEQ ID NO: 52)
HKPGG,
(SEQ ID NO: 53)
KHVPGGG,
(SEQ ID NO: 54)
KHVPGG,
(SEQ ID NO: 55)
HQPGGG,
(SEQ ID NO: 56)
HQPGG
(SEQ ID NO: 801)
VKSKIGST,
(SEQ ID NO: 817)
SKIGSTEN,
(SEQ ID NO: 695)
TENLKHQP,
(SEQ ID NO: 689)
ENLKHQPG,
(SEQ ID NO: 985)
SKIGSTDNIKH,
(SEQ ID NO: 986)
SKIGSKDNIKH,

and
(SEQ ID NO: 988)

SKIGSLDNIKH.

In each of these embodiments, the peptide may comprise, consist, or consist essentially of the recited sequences.

In some embodiments, the Aβ and tau peptides are linked to form a dual Aβ/Tau polypeptide. The Aβ and tau peptides can be linked by an intra-peptide linker. For example, a polypeptide linker located between the C-terminal of the first peptide and the N terminal of the second peptide. With or without the intra-peptide linker, the Aβ peptide and the tau peptide may be positioned in a dual Aβ/tau polypeptide in any order. For example, the Aβ peptide may be positioned at the N-terminal portion of the dual polypeptide and the tau peptide may be positioned at the C-terminal portion of the dual polypeptide. Or, the tau peptide may be positioned at the N-terminal portion of the dual polypeptide and the Aβ peptide may be positioned at the C-terminal portion of the dual polypeptide side of the tau peptide. Reference to a first peptide or a second peptide herein is not intended to suggest an order of the Aβ or tau peptides in the polypeptide of the immunogens.

In addition, the C-terminal portion of the Aβ peptide, the tau peptide, or the dual Aβ-tau polypeptide can include a linker for conjugating the peptides or the polypeptide to a carrier. Linkers that couple the peptides or dual polypeptide to the carrier may include, for example, GG, GGG, KK, KKK, AA, AAA, SS, SSS, GAGA (SEQ ID NO:80), AGAG (SEQ ID NO: 81), KGKG (SEQ ID NO:82), and the like between the peptides or dual polypeptide and the carrier and may further include a C-terminal or a N-terminal cysteine to provide a short peptide linker (e.g., G-G-C-, K-K-C-, A-A-C-, or S-S-C-). In some embodiments, where the C-terminal residues in the immunogen are either IVYKPV (SEQ ID NO:194), VYKPV (SEQ ID NO:195), YKPV (SEQ ID NO:196), KPV, or PV the linker is an amino acid linker that does not have a N-terminal glycine (e.g., GG, GAGA (SEQ ID NO:80)). In some embodiments, the linker comprises an amino acid sequence any one of AA, AAA, KK, KKK, SS, SSS, AGAG (SEQ ID NO: 81), GG, GGG, GAGA (SEQ ID NO:80), and KGKG (SEQ ID NO:82). In some embodiments, any of the Aβ peptide, the tau peptide, and the dual Aβ/tau polypeptide may include a C-terminal cysteine without the spacer. In some embodiments, any of the Aβ peptide, the tau peptide, and the dual Aβ/tau polypeptide may include a N-terminal cysteine without the spacer.

When the Aβ and tau polypeptides are linked to form a dual Aβ/tau polypeptide, the linker may be a cleavable linker. As used herein, the term “cleavable linker” refers to any linker between the antigenic peptides that promotes or otherwise renders the Aβ peptide and the tau peptide more susceptible to separation from each other by cleavage (for example, by endopeptidases, proteases, low pH or any other means that may occur within or around the antigen-presenting cell) and, thereby, processing by the antigen-presenting cell, than equivalent peptides lacking such a cleavable linker. In some compositions, the cleavable linker is a protease-sensitive dipeptide or oligopeptide cleavable linker. In certain embodiments, the cleavable linker is sensitive to cleavage by a protease of the trypsin family of proteases. In some compositions, the cleavable linker comprises an amino acid sequence selected from the group consisting of arginine-arginine (Arg-Arg), arginine-valine-arginine-arginine (Arg-Val-Arg-Arg; SEQ ID NO:69), valine-citrulline (Val-Cit), valine-arginine (Val-Arg), valine-lysine (Val-Lys), valine-alanine (Val-Ala), phenylalanine-lysine (Phe-Lys), GAGA (SEQ ID NO:80), AGAG (SEQ ID NO:81), and KGKG (SEQ ID NO:82). In some compositions, the cleavable linker is arginine-arginine (Arg-Arg).

In some embodiments of the disclosure, the dual Aβ/tau polypeptide comprises, consists or consists essentially of an amino acid sequence selected from DAEFRHDRRQIVYKPV (SEQ ID NO:57) or DAEFRHDRREIVYKSV (SEQ ID NO:58), or DAEFRHDRRQIVYKPVXXC (SEQ ID NO:70), wherein XX and the C-terminal cysteine are each independently optional, or DAEFRHDRREIVYKSVXXC (SEQ ID NO:79), wherein XX and C are independently optional and, if present, XX can be GG, AA, KK, SS, GAGA (SEQ ID NO: 80), AGAG (SEQ ID NO:81), and KGKG (SEQ ID NO:82).

In some embodiments, the dual Aβ/tau polypeptide is as follows:

[first peptide]-[linker 1]-[second peptide]-[linker 2]-[Cys],

wherein, if the [first peptide] is an Aβ peptide then the [second peptide] is a tau peptide, and if the [first peptide] is a tau peptide, then the [second peptide] is an Aβ peptide, each of [linker 1], [linker 2] and [Cys] are optional, and [linker 1] and [linker 2] are the same or different linkers.

In certain embodiments, the dual Aβ/tau polypeptide is as follows:

[Cys]-[linker 2]-[first peptide]-[linker 1]-[second peptide]

wherein, if the [first peptide] is an Aβ peptide then the [second peptide] is a tau peptide, and if the [first peptide] is a tau peptide, then the [second peptide] is an Aβ peptide, and each of [linker 1], [linker 2] and [Cys] are optional, and [linker 1] and [linker 2] are the same or different linkers.

Examples of the Aβ peptide include any one SEQ ID NOS 3-38 or SEQ ID NOS: 1002-1057.

Examples of the tau peptide include any one of SEQ ID NOS: 39-56, 83-86, or 146-996.

[Linker 1] is optional, and when present, may be a cleavable linker. A cleavable linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the cleavable linker is 1 amino acid, 2 amino acids, 3 amino acids, 4 amino acids, 5 amino acids, 6 amino acids, 7 amino acids, 8 amino acids, 9 amino acids, or 10 amino acids. In some embodiments, the linker may be a cleavable linker having an amino acid sequence selected from the group consisting of arginine-arginine (Arg-Arg), arginine-valine-arginine-arginine (Arg-Val-Arg-Arg; SEQ ID NO: 69), valine-citrulline (Val-Cit), valine-arginine (Val-Arg), valine-lysine (Val-Lys), valine-alanine (Val-Ala), phenylalanine-lysine (Phe-Lys), glycine-alanine-glycine-alanine (Gly-Ala-Gly-Ala; SEQ ID NO:80), alanine-glycine-alanine-glycine (Gly-Ala-Gly-Ala; SEQ ID NO:81), and lysine-glycine-lysine-glycine (Lys-Gly-Lys-Gly; SEQ ID NO:82).

[Linker 2] is optional, and when present is a linker that couples the polypeptide to a carrier. A linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the linker is 1 amino acid, 2 amino acids, 3 amino acids, 4 amino acids, 5 amino acids, 6 amino acids, 7 amino acids, 8 amino acids, 9 amino acids, or 10 amino acids. In some embodiments, the amino acid composition of a linker can mimic the composition of linkers found in natural multidomain proteins, where certain amino acids are overrepresented, underrepresented or equi-represented in natural linkers as compared to their abundance in whole protein. For example, threonine (Thr), serine (Ser), proline (Pro), glycine (Gly), aspartic acid (Asp), lysine (Lys), glutamine (Gln), asparagine (Asn), arginine (Arg), phenylalanine (Phe), glutamic acid (Glu) and alanine (Ala) are overrepresented in natural linkers. In contrast, isoleucine (Ile), tyrosine (Tyr), tryptophan (Trp), and cysteine (Cys) are underrepresented. In general, overrepresented amino acids were polar uncharged or charged residues, which constitute approximately 50% of naturally encoded amino acids, and Pro, Thr, and Gln were the most preferable amino acids for natural linkers. In some embodiments, the amino acid composition of a linker can mimic the composition of linkers commonly found in recombinant proteins, which can generally by classified as flexible or rigid linkers. For example, flexible linkers found in recombinant proteins are generally composed of small, non-polar (e.g. Gly) or polar (e.g. Ser or Thr) amino acids whose small size provides flexibility and allows for mobility of the connecting functional domains. The incorporation of, e.g., Ser or Thr can maintain the stability of the linker in aqueous solutions by forming hydrogen bonds with the water molecules, and therefore can reduce interactions between the linker and the immunogens. In some embodiments, a linker comprises stretches of Gly and Ser residues (“GS” linker). An example of a widely used flexible linker is (Gly-Gly-Ser) n, (Gly-Gly-Gly-Ser) n (SEQ ID NO:1062) or (Gly-Gly-Gly-Gly-Ser) n (SEQ ID NO: 1063), where n=1-3. Adjusting the copy number “n” can optimize a linker to achieve sufficient separation of the functional immunogen domains to, e.g., maximize an immunogenic response. Many other flexible linkers have been designed for recombinant fusion proteins that can be used herein. In some embodiments, linkers can be rich in small or polar amino acids such as Gly and Ser but also contain additional amino acids such as Thr and Ala to maintain flexibility, as well as polar amino acids such as Lys and Glu to improve solubility. See, e.g., Chen, X. et al., “Fusion Protein Linkers: Property, Design and Functionality” Adv Drug Deliv Rev., 15; 65(10): 1357-1369 (203). In certain embodiments, when present, the linker can be an amino acid sequence selected from the group consisting of as GG, GGG, KK, KKK, AA, AAA, SS, SSS, G-A-G-A (SEQ ID NO:80), A-G-A-G (SEQ ID NO:81), and K-G-K-G (SEQ ID NO: 82).

[Cys] is optional and can be helpful to conjugate the polypeptide to a carrier. When present, the Cys can be at the C-terminal portion of the polypeptide, or at the N-terminal portion of the polypeptide.

Examples of the [first peptide]-[linker 1]-[second peptide]-[linker 2]-[Cys] dual Aβ/tau polypeptide of the disclosure include the following:

TABLE 1

| | | Abeta | Endo | | tau | | | Immunogen |
|---|---|---|---|---|---|---|---|---|
| Immuno- | | SEQ | pepti- | | SEQ | | | SEQ |
| gen | Abeta | ID | dase | Tau | ID | C-Terminal | | ID |
| Lab ID | Sequence | NO. | linker | Sequence | NO. | linker | Cys | NO |
| 9 | DAEFRHD | 06 | RR | QIVYKPV | 39 | GG | C | 59 |
| 10 | DAEFRHD | 06 | RR | QIVYKPV | 39 | None | C | 60 |
| 18 | DAEFRHD | 06 | RR | QIVYKPV | 39 | AA | C | 61 |
| 19 | DAEFRHD | 06 | RR | QIVYKPV | 39 | KK | C | 62 |
| 20 | DAEFRHD | 06 | RR | EIVYKSP | 43 | GG | C | 63 |
| 21 | DAEFRHD | 06 | RR | EIVYKSP | 43 | AA | C | 64 |
| 22 | DAEFRHD | 06 | RR | EIVYKSP | 43 | None | C | 65 |
| 23 | DAEFRHD | 06 | RR | EIVYKSP | 43 | KK | C | 66 |
| 12 | DAEFRHD | 06 | RR | NIKHVP | 48 | GG | C | 78 |

### Polypeptide Immunogens

The Aβ peptide, the tau peptide and, the dual Aβ/tau polypeptide are immunogens in accordance with the disclosure. In some embodiments, the peptides and the dual Aβ-tau polypeptide can be linked to a suitable carrier to help elicit an immune response. Accordingly, one or more the peptides and dual Aβ-tau polypeptides of the disclosure can be linked to a carrier. For example, each of the Aβ peptide, tau peptide and the Aβ-tau polypeptide may be linked to the carrier with or without spacer amino acids (e.g., Gly-Gly, Gly-Gly-Gly, Ala-Ala, Ala-Ala-Ala, Lys-Lys, Lys-Lys-Lys, Ser-Ser, Ser-Ser-Ser, Gly-Ala-Gly-Ala (SEQ ID NO:80), Ala-Gly-Ala-Gly (SEQ ID NO:81), or Lys-Gly-Lys-Gly (SEQ ID NO:82)). In certain embodiments, the dual Aβ-tau polypeptide can be linked to a suitable carrier using a C-terminal cysteine to provide a linker between the peptide(s) and the carrier or the dual Aβ/tau polypeptide and the carrier. In certain embodiments, the dual Aβ-tau polypeptide can be linked to a suitable carrier using an N-terminal cysteine to provide a linker between the peptide(s) and the carrier. In some embodiments, where the C-terminal residues in the immunogen are either IVYKPV (SEQ ID NO: 194), VYKPV (SEQ ID NO:195), YKPV (SEQ ID NO:196), KPV, or PV the linker is an amino acid linker that does not have a N-terminal glycine (e.g., GG, GAGA (SEQ ID NO:80)).

Suitable carriers include, but are not limited to serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking peptide immunogens of the invention to an immunostimulatory polymer molecule (e.g., tripalmitoyl-S-glycerine cysteine (Pam3Cys), mannan (a mannose polymer), or glucan (a β 1-2 polymer)), cytokines (e.g., IL-1, IL-1 alpha and β peptides, IL-2, γ-INF, IL-10, GM-CSF), and chemokines (e.g., MIP1-α and β, and RANTES). Additional carriers include virus-like particles. In some compositions, immunogenic peptides can also be linked to carriers by chemical crosslinking. Techniques for linking an immunogen to a carrier include the formation of disulfide linkages using N-succinimidyl 3-(2-pyridylthio) propionate (SPDP), and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine resides on one protein and an amide linkage through the epsilon-amino on a lysine, or other free amino group in other amino acids. In some embodiments, chemical crosslinking can comprise use of SBAP (succinimidyl 3-(bromoacetamido) propionate), which is a short (6.2 angstrom) cross-linker for amine-to-sulfhydryl conjugation via N-hydroxysuccinimide (NHS) ester and bromoacetyl reactive groups. A variety of such disulfide/amide-forming agents are described by Jansen et al., “Immunotoxins: Hybrid Molecules Combining High Specificity and Potent Cytotoxicity” Immunological Reviews 62:185-216 (February 1982). Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimido-methyl) cy-clohexane-1-carboxylic acid. The carboxyl groups can be activated by combining them with succinimide or 1-hydroxyl-2-nitro-4-sulfonic acid, sodium salt. Virus-like particles (VLPs), also called pseudovirions or virus-derived particles, represent subunit structures composed of multiple copies of a viral capsid and/or envelope protein capable of self-assembly into VLPs of defined spherical symmetry in vivo. (Powilleit, et al., (2007) PLOS ONE 2(5):e415.) Alternatively, peptide immunogens can be linked to at least one artificial T-cell epitope capable of binding a large proportion of MHC Class II molecules, such as the pan DR epitope (“PADRE”). Pan DR-binding peptides (PADRE) are described in U.S. Pat. No. 5,736,142, WO 95/07707, and Alexander, et al, Immunity, 1:751-761 (1994).

Active immunogens can be presented in multimeric form in which multiple copies of an immunogen (peptide of polypeptide) are presented on a carrier as a single covalent molecule. In some embodiments, the carrier includes various forms of the dual Aβ/tau polypeptide. For instance, the dual Aβ/tau polypeptide of the immunogen can include polypeptides that have the Aβ antigen and the tau antigen in different orders, or may be present with or without an intrapeptide linker and/or a linker to a carrier.

In some compositions, the immunogenic peptides can also be expressed as fusion proteins with carriers. In certain compositions, the immunogenic peptides can be linked at the amino terminus, the carboxyl terminus, or internally to the carrier. In some compositions, the carrier is CRM197. In some compositions, the carrier is diphtheria toxoid.

### Nucleic Acids

The disclosure further provides nucleic acids encoding any of the amyloid-beta (Aβ) peptides and the tau peptides as disclosed herein. The nucleic acid immunotherapy compositions, as disclosed herein, comprise, consist of, or consist essentially of, a first nucleic acid sequence encoding an amyloid-beta (Aβ) peptide, and a second nucleic acid sequence encoding a tau peptide as disclosed herein. For example, the Aβ peptide is a sequence that is 3-10 amino acid residues in length and from the first ten N-terminal residues of SEQ ID NO:01, and the tau peptide is a sequence that is 3-13 amino acids in length and from residues 244-400 of SEQ ID NO:02. Accordingly, a nucleic acid encoding any of SEQ ID NOS: 3-38 or SEQ ID NOS: 1002-1057 may be combined with a nucleic acid encoding any of SEQ ID NOS: 39-56, 83-86, or 146-996 to provide an immunogen and a component of pharmaceutical composition of the disclosure. Likewise, one or more nucleic acids encoding any of Abeta and tau sequences may include the codons for an RR- N-terminal or -RR C-terminal dipeptide. In certain embodiments, the Aβ and tau peptide sequences may be encoded by the same nucleic acid sequence or by separate nucleic acid sequences. In some embodiments, the nucleic acid sequences may also encode a linker to a carrier and/or a C-terminal cysteine as described herein. In addition, when a single nucleic acid sequence encodes both peptides, the sequence may also encode an intra-peptide linker as described herein. The nucleic acid compositions described herein (pharmaceutical compositions) can be used in methods for treating or effecting prophylaxis and/or prevention of Alzheimer's disease. In another embodiment, the nucleic acid immunotherapy compositions as disclosed herein provide compositions for reducing pathogenic forms of Aβ and/or tau in the subject and/or in the tissue of the subject. In some embodiments, the Aβ and/or tau reduced by the immunotherapy compositions is the pathological form(s) of the Aβ (e.g. extracellular plaque deposits of the β-amyloid peptide (Aβ); neuritic amyloid plaques), and/or tau (e.g. flame-shaped neurofibrillary tangles of tau; neurofibrillary tangles of tau). In yet other embodiment, pathological indicators of neurodegenerative disease are decreased by the nucleic acid immunotherapy compositions. In another embodiment, the nucleic acid immunotherapy compositions as disclosed herein provide compositions for reducing brain Aβ and brain tau.

A nucleic acid such as DNA that encodes an immunogen and is used as a vaccine can be referred to as a “DNA immunogen” or “DNA vaccine” as the encoded polypeptides are expressed in vivo after administration of the DNA. DNA vaccines are intended to induce antibodies against the proteins of interest they encode in a subject by: integrating DNA encoding the proteins of interest into a vector (a plasmid or virus); administering the vector to the subject; and expressing the proteins of interest in the subject in which the vector has been administered to stimulate the immune system of the subject. A DNA vaccine remains in the body of the subject for a long time after the administration, and continues to slowly produce the encoded proteins. Thus, excessive immune responses can be avoided. DNA vaccines can also be modified using a genetic engineering techniques. Optionally, such nucleic acids further encode a signal peptide and can be expressed with the signal peptide linked to peptide. Coding sequences of nucleic acids can be operably linked with regulatory sequences to ensure expression of the coding sequences, such as a promoter, enhancer, ribosome binding site, transcription termination signal, and the like. The nucleic acids encoding Aβ and tau can occur in isolated form or can be cloned into one or more vectors. The nucleic acids can be synthesized by, for example, solid state synthesis or PCR of overlapping oligonucleotides. Nucleic acids encoding Aβ and tau peptides and polypeptides with and without linkers or cleavable linkers and with our without protein based carriers can be joined as one contiguous nucleic acid, e.g., within an expression vector.

DNA is more stable than RNA, but involves some potential safety risks such as induction of anti-DNA antibodies, thus in some embodiments, the nucleic acid can be RNA. RNA nucleic acid that encodes an immunogen and is used as a vaccine can be referred to as a “RNA immunogen” or “RNA vaccine” or “mRNA vaccine” as the encoded polypeptides are expressed in vivo after administration of the RNA. Ribonucleic acid (RNA) vaccines can safely direct a subject's cellular machinery to produce one or more polypeptide(s) of interest. In some embodiments, a RNA vaccine can be a non-replicating mRNA (messenger-RNA) or a virally derived, self-amplifying RNA. mRNA-based vaccines encode the antigens of interest and contain 5′ and 3′ untranslated regions (UTRs), whereas self-amplifying RNAs encode not only the antigens, but also the viral replication machinery that enables intracellular RNA amplification and abundant protein expression. In vitro transcribed mRNA can be produced from a linear DNA template using a T7, a T3 or an Sp6 phage RNA polymerase. The resulting product can contain an open reading frame that encodes the peptides of interest as disclosed herein, flanking 5′- and 3′-UTR sequences, a 5′ cap and a poly(A) tail. In some embodiments, a RNA vaccine can comprise trans-amplifying RNA (for example, see Beissert et al., Molecular Therapy January 2020 28(1):119-128). In certain embodiments, RNA vaccines encode an Aβ peptide and a tau peptide as disclosed herein, and are capable of expressing the Aβ and a tau peptides, in particular if transferred into a cell such as an immature antigen presenting cell. RNA may also contain sequences which encode other polypeptide sequences such as immune stimulating elements. In some embodiments, the RNA of a RNA vaccine can be modified RNA. The term “modified” in the context of the RNA can include any modification of RNA which is not naturally present in RNA. For example, modified RNA can refer to RNA with a 5′-cap; however, RNA may comprise further modifications. A 5′-cap can be modified to possess the ability to stabilize RNA when attached thereto. In certain embodiments, a further modification may be an extension or truncation of the naturally occurring poly(A) tail or an alteration of the 5′- or 3′-untranslated regions (UTR). In some embodiments, the RNA e.g. or mRNA vaccine is formulated in an effective amount to produce an antigen specific immune response in a subject. For example, the RNA vaccine formulation is administered to a subject in order to stimulate the humoral and/or cellular immune system of the subject against the Aβ and tau antigens, and thus may further comprise one or more adjuvant(s), diluents, carriers, and/or excipients, and is applied to the subject in any suitable route in order to elicit a protective and/or therapeutic immune reaction against the Aβ and tau antigens.

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J D et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

Techniques for the manipulation of nucleic acids, such as, e.g., generating mutations in sequences, sub-cloning, labeling probes, sequencing, hybridization and the like are well described in the scientific and patent literature. See, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immunofluorescence assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

### Pharmaceutical Compositions

Each of the peptides and immunogens described herein can be presented in a pharmaceutical composition that is administered with pharmaceutically acceptable adjuvants and pharmaceutically acceptable excipients. The adjuvant increases the titer of induced antibodies and/or the binding affinity of induced antibodies relative to the situation if the peptide were used alone. A variety of adjuvants can be used in combination with an immunogen of the disclosure to elicit an immune response. Some adjuvants augment the intrinsic response to an immunogen without causing conformational changes in the immunogen that affect the qualitative form of the response. An adjuvant may be a natural compound, a modified version of or derivative of a natural compound, or a synthetic compound.

Some adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). As used herein, MPL refers to natural and synthetic versions of MPL. Examples of synthetic versions include PHAD®, 3D-PHAD® and 3D(6A)-PHAD® (Avanti Polar Lipids, Alabaster, Alabama).

QS-21 is a triterpene glycoside or saponin isolated from the bark of the Quillaja saponaria Molina tree found in South America (see Kensil et al., in Vaccine Design: The Subunit and Adjuvant Approach (eds. Powell & Newman, Plenum Press, NY, 1995)) QS-21 products include Stimulon® (Antigenics, Inc., New York, NY; now Agenus, Inc. Lexington, MA) and QS-21 Vaccine Adjuvant (Desert King, San Diego, CA). QS-21 has been disclosed, characterized, and evaluated in U.S. Pat. Nos. 5,057,540, and 8,034,348, the disclosures of which are herein incorporated by reference. Additionally, QS-21 has been evaluated in numerous clinical trials in various dosages. See, NCT00960531 (clinicaltrials.gov/ct2/show/study/NCT00960531), Hüll et al., Curr Alzheimer Res. 2017 July; 14(7): 696-708 (evaluated 50 mcg of QS-21 in with various doses of vaccine ACC-001); Gilman et al., “Clinical effects of Abeta immunization (AN1792) in patients with AD in an interrupted trial” Neurology. 2005 May 10; 64(9):1553-62; Wald et al., “Safety and immunogenicity of long HSV-2 peptides complexed with rhHsc70 in HSV-2 seropositive persons” Vaccine 2011; 29(47):8520-8529; and Cunningham et al., “Efficacy of the Herpes Zoster Subunit Vaccine in Adults 70 Years of Age or Older.” NEJM. 2016 Sep. 15; 375(11):1019-32. QS-21 is used in FDA approved vaccines including SHINGRIX. SHINGRIX contains 50 mcg of QS-21. In certain embodiments, the amount of QS-21 is from about 10 μg to about 500 μg.

TQL1055 is an analogue of QS-21 (Adjuvance Technologies, Lincoln, NE). The semi-synthetic TQL1055 has been characterized in comparison to QS-21 as having high purity, increased stability, decreased local tolerability, decreased systemic tolerability. TQL1055 has been disclosed, characterized, and evaluated in US20180327436 A1, WO2018191598 A1, WO2018200656 A1, and WO2019079160 A1, the disclosures of which are herein incorporated by reference. US20180327436 A1 teaches that 2.5 fold more TQ1055 was superior to 20 μg QS-21 but there was not an improvement over 50 μg TQ1055. However, unlike QS-21 there was no increase in either weight loss or hemolysis of RBC as the TQL1055 dose increased. WO2018200656 A1 teaches that with an optimal amount of TQ1055, one can lower the amount of antigen and achieve superior titers. In certain embodiments, the amount of TQL1055 is from about 10 μg to about 500 μg.

Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween 80. Ribi also contains refined mycobacterial products which act as immunostimulants and bacterial monophosphoryl lipid A. Other adjuvants can be CpG oligonucleotides (see WO 98/40100), cytokines (e.g., IL-1, IL-1 alpha and β peptides, IL-2, γ-INF, IL-10, GM-CSF), chemokines (e.g., MIP1-α and β, and RANTES), saponins, RNA, and/or TLR agonists (for example, TLR4 agonists such as MPL and synthetic MPL molecules), aminoalkyl glucosaminide phosphate and other TLR agonists. Adjuvants can be administered as a component of a therapeutic composition with an active agent or can be administered separately, before, concurrently with, or after administration of the therapeutic agent.

In various embodiments of the disclosure, the adjuvant is QS-21 (Stimulon™). In some compositions, the adjuvant is MPL. In certain embodiments, the amount of MPL is from about 10 μg to about 500 μg. In some compositions, the adjuvant is TQL1055. In certain embodiments, the amount of TQL1055 is from about 10 μg to about 500 μg. In some compositions, the adjuvant is QS21. In certain embodiments, the amount of QS21 is from about 10 μg to about 500 μg. In some compositions, the adjuvant is a combination of MPL and QS-21. In some compositions, the adjuvant is a combination of MPL and TQL1055. In some compositions, the adjuvant can be in a liposomal formulation.

In addition, some embodiments of the disclosure can comprise a multiple antigen presenting system (MAP). Multiple antigen-presenting peptide vaccine systems have been developed to avoid the adverse effects associated with conventional vaccines (i.e., live-attenuated, killed or inactivated pathogens), carrier proteins and cytotoxic adjuvants. Two main approaches have been used to develop multiple antigen presenting peptide vaccine systems: (1) the addition of functional components, e.g., T-cell epitopes, cell-penetrating peptides, and lipophilic moieties; and (2) synthetic approaches using size-defined nanomaterials, e.g., self-assembling peptides, non-peptidic dendrimers, and gold nanoparticles, as antigen-displaying platforms. Use of a multiple antigenic peptide (MAP) system can improve the sometimes poor immunogenicity of subunit peptide vaccines. In a MAP system, multiple copies of antigenic peptides are simultaneously bound to the a- and ε-amino groups of a non-immunogenic Lys-based dendritic scaffold, helping to confer stability from degradation, thus enhancing molecular recognition by immune cells, and induction of stronger immune responses compared with small antigenic peptides alone. In some compositions, the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the peptides of the disclosure can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, peptide compositions can be in lyophilized form for constitution with a suitable vehicle, e.g., sterile pyrogen-free water, before use.

Peptides (and optionally a carrier fused to the peptide(s)) can also be administered in the form of a nucleic acid encoding the peptide(s) and expressed in situ in a subject. A nucleic acid segment encoding an immunogen is typically linked to regulatory elements, such as a promoter and enhancer that allow expression of the DNA segment in the intended target cells of a subject. For expression in blood cells, as is desirable for induction of an immune response, promoter and enhancer elements from, for example, light or heavy chain immunoglobulin genes or the CMV major intermediate early promoter and enhancer are suitable to direct expression. The linked regulatory elements and coding sequences are often cloned into a vector.

DNA and RNA can be delivered in naked form (i.e., without colloidal or encapsulating materials). Alternatively a number of viral vector systems can be used including retroviral systems (see, e.g., Boris-Lawrie and Teumin, Cur. Opin. Genet. Develop. 3(1):102-109 (1993)); adenoviral vectors (see, e.g., Bett et al, J. Virol. 67(10); 5911-21 (1993)); adeno-associated virus vectors (see, e.g., Zhou et al., J. Exp. Med. 179(6):1867-75 (1994)), viral vectors from the pox family including vaccinia virus and the avian pox viruses, viral vectors from the alpha virus genus such as those derived from Sindbis and Semliki Forest Viruses (see, e.g., Dubensky et al., J. Virol. 70(1):508-519 (1996)), Venezuelan equine encephalitis virus (see U.S. Pat. No. 5,643,576) and rhabdoviruses, such as vesicular stomatitis virus (see WO 96/34625) and papillomaviruses (WO 94/12629; Ohe et al., Human Gene Therapy 6(3):325-333 (1995); and Xiao & Brandsma, Nucleic Acids. Res. 24(13):2620-2622 (1996)).

DNA and RNA encoding an immunogen, or a vector containing the same, can be packaged into liposomes, nanoparticles or lipoproteins complexes. Suitable other polymers, include, for example, protamine liposomes, polysaccharide particles, cationic nanoemulsion, cationic polymer, cationic polymer liposome, cationic lipid nanoparticles, cationic lipid, cholesterol nanoparticles, cationic lipid-cholesterol, PEG nanoparticle, or dendrimer nanoparticles. Additional suitable lipids and related analogs are described by U.S. Pat. Nos. 5,208,036, 5,264,618, 5,279,833, and 5,283,185, each of which are herein incorporated by reference in their entirety. Vectors and DNA encoding an immunogen can also be adsorbed to or associated with particulate carriers, examples of which include polymethyl methacrylate polymers and polylactides and poly(lactide-co-glycolides), (see, e.g., McGee et al., J. Micro Encap. March-April 1997; 14(2):197-210).

Pharmaceutically acceptable carrier compositions can also include additives, including, but not limited to, water, pharmaceutically acceptable organic solvents, collagen, polyvinyl alcohol, polyvinylpyrrolidone, carboxyvinyl polymers, carboxymethylcellulose sodium, sodium polyacrylate, sodium alginate, water-soluble dextran, carboxymethyl starch sodium, pectin, methylcellulose, ethylcellulose, xanthan gum, gum arabic, casein, agar, polyethylene glycol, diglycerine, glycerine, propylene glycol, petrolatum, paraffin, stearyl alcohol, stearic acid, human serum albumin, mannitol, sorbitol, lactose, and surfactants acceptable as pharmaceutical additives.

### Subjects Amenable to Treatment

The presence of Aβ plaques and/or neurofibrillary tangles has been found in several diseases including Alzheimer's disease, Down's syndrome, mild cognitive impairment, cerebral amyloid angiopathy, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer's disease (LBVAD), chronic traumatic encephalopathy (CTE), globular glial tauopathy (GGT), Parkinson's disease, progressive supranuclear palsy (PSP), dry age-related macular degeneration (AMD), and inclusion-body myositis.

The compositions and methods of the disclosure can be used in treatment or prophylaxis of any of these diseases. Because of the widespread association between neurological diseases and Aβ and/or tau, the compositions and methods of the disclosure can be used in treatment or prophylaxis of any subject showing elevated levels of Aβ and/or tau (e.g., in the CSF) compared with a mean value in individuals without neurological disease. The compositions and methods of the disclosure can also be used in treatment or prophylaxis of neurological disease in individuals having a mutation in Aβ and/or tau associated with neurological disease. The methods are particularly suitable for treatment or prophylaxis of Alzheimer's disease.

Subjects amenable to treatment include individuals at risk of disease but not showing symptoms, as well as patients presently showing symptoms, including treatment naïve subjects that have not been previous treated for disease. Subjects at risk of disease include those in an aging population, asymptomatic subjects with Aβ and/or tau pathologies and having a known genetic risk of disease. Such individuals include those having relatives who have experienced this disease, and those whose risk is determined by analysis of genetic or biochemical markers. Genetic markers of risk include mutations in Aβ and/or tau, as well as mutations in other genes associated with neurological disease. For example, the ApoE4 allele in heterozygous and even more so in homozygous form is associated with risk of Alzheimer's disease (AD). Other markers of risk of Alzheimer's disease include mutations in the APP gene, particularly mutations at position 717 and positions 670 and 671 referred to as the Hardy and Swedish mutations respectively, mutations in the presenilin genes, PS1 and PS2, a family history of AD, hypercholesterolemia or atherosclerosis. Individuals presently suffering from Alzheimer's disease can be recognized by PET imaging, from characteristic dementia, as well as the presence of risk factors described above. In addition, a number of diagnostic tests are available for identifying individuals who have AD. These include measurement of CSF or blood tau or phospho-tau and Aβ42 levels. Elevated tau or phospho-tau and decreased Aβ42 levels signify the presence of AD. Some mutations associated with Parkinson's disease, for example, Ala30Pro or Ala53Thr, or mutations in other genes associated with Parkinson's disease such as leucine-rich repeat kinase (LRRK2 or PARK8). Subjects can also be diagnosed with any of the neurological diseases mentioned above by the criteria of the DSM IV TR.

In asymptomatic subjects, treatment can begin at any age (e.g., 10, 20, 30, or more). Usually, however, it is not necessary to begin treatment until a subject reaches 20, 30, 40, 50, 60, 70, 80, or 90 years of age. Treatment typically entails multiple dosages over a period of time. Treatment can be monitored by assaying antibody levels over time. If the response falls, a booster dosage is indicated. In the case of potential Down's syndrome patients, treatment can begin antenatally by administering therapeutic agent to the mother or shortly after birth.

### Methods of Treatments and Uses

The disclosure provides methods of inhibiting or reducing aggregation of Abeta and/or tau in a subject having or at risk of developing a neurodegenerative disease (e.g., Alzheimer's disease). The methods include administering to the subject the compositions as disclosed herein. A therapeutically effective amount is a dosage that, when given for an effective period of time, achieves the desired immunological or clinical effect. Dosage regimens may be adjusted to provide the optimum therapeutic response. For example, several divided doses may be administered at set intervals (e.g., weekly, monthly) or the dose may be proportionally reduced as indicated by the exigencies of the therapeutic situation.

In prophylactic applications, the compositions described herein can be administered to a subject susceptible to, or otherwise at risk of a disease (e.g., Alzheimer's disease) in a regimen (dose, frequency and route of administration) effective to reduce the risk, lessen the severity, or delay the onset of at least one sign or symptom of the disease. In particular, the regimen is effective to inhibit or delay Aβ plaque formation and/or inhibit or delay tau or phospho-tau and paired filaments formed from it in the brain, and/or inhibit or delay its toxic effects and/or inhibit/or delay development of behavioral deficits. In therapeutic applications, the compositions described herein are administered to a subject suspected of, or a patient already suffering from a disease (e.g., Alzheimer's disease) in a regimen (dose, frequency and route of administration) effective to ameliorate or at least inhibit further deterioration of at least one sign or symptom of the disease. In particular, the regimen is preferably effective to reduce or at least inhibit further increase of levels of Aβ plaques and/or tau, phosphor-tau, or paired filaments formed from it, associated toxicities and/or behavioral deficits.

A regimen is considered therapeutically or prophylactically effective if an individual treated achieves an outcome more favorable than the mean outcome in a control population of comparable subjects not treated by methods of the invention, or if a more favorable outcome is demonstrated in treated subjects versus control subjects in a controlled clinical trial (e.g., a phase II, phase II/III or phase III trial) at the p<0.05 or 0.01 or even 0.001 level.

Effective doses of vary depending on many different factors, such as means of administration, target site, physiological state of the patient, whether the patient is an ApoE carrier, whether the patient is human or an animal, other medications administered, and whether treatment is prophylactic or therapeutic.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times. In some embodiments, a RNA (e.g., mRNA) vaccine is administered to a subject by intradermal, intramuscular injection, or by intranasal administration.

In some embodiments, the amount of an agent for active immunotherapy varies from 1 to 1,000 micrograms (μg), or from 0.1-500 μg, or from 10 to 500 μg, or from 50 to 250 μg per patient and can be from 1-100 or 1-10 μg per injection for human administration. The timing of injections can vary significantly from once a day, to once a week, to once a month, to once a year, to once a decade. A typical regimen consists of an immunization followed by booster injections at time intervals, such as 6 week intervals or two months. Another regimen consists of an immunization followed by one or more booster injections 1, 2, 3, 4, 5, 6, 7, 8, 9, 10, 11, or 12 months later. Another regimen entails an injection every two months for life. Alternatively, booster injections can be on an irregular basis as indicated by monitoring of immune response. The frequency of administration may be once or more as long as the side effects are within a clinically acceptable range.

In some embodiments, the compositions or methods as disclosed herein comprise administering to a subject a nucleic acid vaccine comprising one or more DNA or RNA polynucleotides having an open reading frame encoding a first peptide and a second peptide wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid is administered to the subject by intradermal or intramuscular injection. In some embodiments, the nucleic acid is administered to the subject on day zero. In some embodiments, a second dose of the nucleic acid is administered to the subject on day seven, or fourteen, or twenty one.

The compositions described herein are preferably administered via a peripheral route (i.e., one in which the administered composition results in a robust immune response and/or the induced antibody population crosses the blood brain barrier to reach an intended site in the brain, spinal cord, or eye). For peripheral diseases, the induced antibodies leave the vasculature to reach the intended peripheral organs. Routes of administration include oral, subcutaneous, intranasal, intradermal, or intramuscular. Some routes for active immunization are subcutaneous and intramuscular. Intramuscular administration and subcutaneous administration can be made at a single site or multiple sites. Intramuscular injection is most typically performed in the arm or leg muscles. In some methods, agents are injected directly into a particular tissue where deposits have accumulated.

The number of dosages administered can be adjusted to result in a more robust immune response (for example, higher titers). For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. For chronic disorders, a vaccine/immunotherapy as disclosed herein can be administered at regular intervals, e.g., weekly, fortnightly, monthly, quarterly, every six months for at least 1, 5 or 10 years, or the life of the patient.

An effective amount of a DNA or RNA encoded immunogen can be between about 1 nanogram and about 1 gram per kilogram of body weight of the recipient, or about between about 0.1 μg/kg and about 10 mg/kg, or about between about 1 μg/kg and about 1 mg/kg. Dosage forms suitable for internal administration preferably contain (for the latter dose range) from about 0.1 μg to 100 μg of active ingredient per unit. The active ingredient may vary from 0.5 to 95% by weight based on the total weight of the composition. Alternatively, an effective dose of dendritic cells loaded with the antigen is between about 10⁴ and 10⁸ cells. Those skilled in the art of immunotherapy will be able to adjust these doses without undue experimentation.

The nucleic acid compositions may be administered in a convenient manner, e.g., injection by a convenient and effective route. Routes can include, but are not limited to, intradermal “gene gun” delivery or intramuscular injection. The modified dendritic cells are administered by subcutaneous, intravenous or intramuscular routes. Other possible routes include oral administration, intrathecal, inhalation, transdermal application, or rectal administration.

Depending on the route of administration, the composition may be coated in a material to protect the compound from the action of enzymes, acids and other natural conditions which may inactivate the compound. Thus, it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol) or in an appropriate carrier such as liposomes (including water-in-oil-in-water emulsions) as well as conventional liposomes (Strejan et al., J. Neuroimmunol 7(1):27-41, 1984).

The immunotherapeutic compositions disclosed herein may also be used in combination with other treatments for diseases associated with the accumulation of Aβ or tau, for example, anti-Aβ antibodies such as antibodies that specifically bind to any of the Aβ epitopes disclosed herein. For example, aducanumab or any of the antibodies disclosed in, for example, U.S. Patent Publication No. 20100202968 and U.S. Pat. No. 8,906,367, and/or anti-tau antibodies such as antibodies that specifically bind to any of the tau epitopes disclosed herein, ABBV-8E12, gosuranemab, zagotenemab, RG-6100, BIIB076 or any of the antibodies disclosed in WO2014/165271, U.S. Pat. No. 10,501,531, WO2017/191559, WO2017/191560, WO2017/191561, US20190330314, US20190330316, and WO2018/204546. In some combination therapy methods, the patient receives passive immunotherapy prior to the active immunotherapy methods disclosed herein. In other methods, the patient receives passive and active immunotherapy during the same period of treatment. Alternatively, patients may receive active immunotherapy prior to passive immunotherapy. Combinations may also include small molecule therapies and non-immunogenic therapies such as RAZADYNE® (galantamine), EXELON® (rivastigmine), and ARICEPT® (donepezil) and other compositions that improve the function of nerve cells in the brain.

The compositions of the disclosure may be used in the manufacture of medicaments for the treatment regimens described herein.

### Treatment Regimens

Desired outcomes of the methods of treatment as disclosed herein vary according to the disease and patient profile and are determinable to those skilled in the art. Desired outcomes include an improvement in the patient's health status. Generally, desired outcomes include measurable indices such as reduction or clearance of pathologic amyloid fibrils, decreased or inhibited amyloid aggregation and/or deposition of amyloid fibrils, and increased immune response to pathologic and/or aggregated amyloid fibrils. Desired outcomes also include amelioration of amyloid disease-specific symptoms. As used herein, relative terms such as “improve,” “increase,” or “reduce” indicate values relative to a control, such as a measurement in the same individual prior to initiation of treatment described herein, or a measurement in a control individual or group. A control individual is an individual afflicted with the same amyloid disease as the individual being treated, who is about the same age as the individual being treated (to ensure that the stages of the disease in the treated individual and the control individual are comparable), but who has not received treatment using the disclosed immunotherapy/vaccine formulations. Alternatively, a control individual is a healthy individual, who is about the same age as the individual being treated. Changes or improvements in response to therapy are generally statistically significant and described by a p-value less than or equal to 0.1, less than 0.05, less than 0.01, less than 0.005, or less than 0.001 may be regarded as significant.

Effective doses of the compositions as disclosed herein, for the treatment of a subject vary depending upon many different factors, including means of administration, target site, physiological state of the patient, whether the patient is human or an animal, other medications administered, if any, and whether treatment is prophylactic or therapeutic. Treatment dosages can be titrated to optimize safety and efficacy. The amount of immunogen can also depend on whether adjuvant is also administered, with higher dosages being required in the absence of adjuvant. The amount of an immunogen for administration sometimes varies from 1-500 μg per patient and more usually from 5-500 μg per injection for human administration. Occasionally, a higher dose of 1-2 mg per dosage is used. Typically, about 10, 20, 50 or 100 μg is used for each human dosage. The timing of dosages can vary significantly from once a day, to once a year, to once a decade. On any given day that a dosage of immunogen is given, the dosage is greater than 1 μg/patient and usually greater than 10 μg/patient if adjuvant is also administered, and greater than 10 μg/patient and usually greater than 100 μg/patient in the absence of adjuvant. A typical regimen consists of an immunization followed by booster dosage(s) at 6-week intervals. Another regimen consists of an immunization followed by booster dosage(s) 1, 2, 3, 4, 5, 6, 7, 8, 9, 10, 11, or 12 months later. Another regimen entails dosage(s) every two months for life. Alternatively, booster dosage(s) can be on an irregular basis as indicated by monitoring of immune response.

When administered in combination with a second treatment for Alzheimer's disease, such as, Razadyne® (galantamine), Exelon® (rivastigmine), and Aricept® (donepezil), the second treatment can be administered according the product label or as necessary in view of the treatment with the compositions of the disclosure.

### Kits

The disclosure further provides kits (e.g., containers) comprising the compositions disclosed herein and related materials, such as instructions for use (e.g., package insert). The instructions for use may contain, for example, instructions for administration of the compositions and optionally one or more additional agents. The containers of peptide and/or nucleic acid compositions may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products. Kits can also include a second container comprising a pharmaceutically acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

### Uses

Each of the peptides, polypeptides, immunogens, and pharmaceutical compositions described herein may be for use in treating one or more of the diseases as described herein. In addition, each of the peptides, polypeptides, immunogens, and pharmaceutical compositions described herein may be for use in methods for treating one or more of the diseases as described herein. Each of the peptides, polypeptides, immunogens, and pharmaceutical compositions described herein may be used in a method for manufacturing a medicament for treating or use in treating one or more of the diseases as described herein.

The following are provided for exemplification purposes only and are not intended to limit the scope of the invention described in broad terms above.

All U.S. and international patent applications identified herein are incorporated by reference in their entirety.

### EXAMPLES

### Example 1: Immunization of Guinea Pigs

Guinea pigs were injected intramuscularly with 50 μg of a test immunogen, 25 μg QS21 in 200 μl of Addavax on day 0, 21, 49 and 77. Bleeds were done 7 days post immunization. The peptides tested included DAEFRHD (SEQ ID NO:06), QIVYKPV (SEQ ID NO: 39), and DAEFRHDRRQIVYKPV (SEQ ID NO:57). The specific immunogens were DAEFRHDC (SEQ ID NO:71), QIVYKPVGGC (SEQ ID NO:72), and DAEFRHDRRQIVYKPVGGC (SEQ ID NO:59). The peptides were coupled through the C-terminal cysteine to CRM-197 with a maleimide linkage.

Female Guinea Pigs were at least 5 weeks old at the start of the study having an approximate body weight of 350-500 g. Appropriate animal housing and research procedures for animal husbandry and care were conducted in an accredited facility in accordance with the guidelines of the U.S. Department of Agriculture's (USDA) and the Assessment and Accreditation of Laboratory Animal Care (AAALAC) International.

The immunogen concentration was 0.5 mg/ml. Prior to each administration of the test immunogen, approximately a 3 cm² area on each hind limb was shaved and wiped with ethanol for visualization of the injection site. Each animal received a test immunogen dose of 200 microliters (0.25 micrograms/microliter) divided into two separate sites each of 100 microliter per injection (i.e., animals received 50 μg of immunogen in 100 μl PBS+25 μg of QS21 in 100 μl MF59). A 25 G-27 G needle was inserted intramuscularly into the hind limb, approximately 0.25-0.5 cm deep, and injected at 100 microliters per site. Injection sites were rotated each administration between four separate sites per hind limb and separated by at least 2 cm.

### Example 2: Measurement of Antibody Titers

Whole blood samples were collected into clot activator tubes via jugular vein at 250-350 microliters per collection at weeks 1, 4, and 8. The maximum volume of whole blood was collected into clot activator tubes via cardiac puncture at termination on week 12. All blood samples were allowed to clot at room temperature for greater than 30 minutes, centrifuged at ambient temperature (approximately 20-25° C.) at 3,000 RPM for 10-15 minutes, and serum supernatant was transferred individually into clean cryovials. Serum supernatant was stored frozen at −80° C. (+12° C.).

### Titer Guinea Pig Bleed on Soluble Aβ Aggregates

2.5 μg/ml of a soluble aggregate Aβ prep (HFIP film of Aβ 42 was resuspended and incubated overnight with shaking, then spun to remove insoluble aggregate) was coated on to the plate at 100 μl per well in PBS and incubated overnight at room temperature. Plates were blocked for 1 hour with 1% BSA in PBS. Plates were aspirated and to row A, 200 μl of 0.1% BSA in PBS Tween was added to 1-4. In 1 neg. GP serum was added at 1/100 while 2-4 contained 1/100 test serum. Rows B-H contained 100 μl of 0.1% BSA in PBS Tween. Rows were serially diluted ½ down the plate giving dilution of 1/100 to 1/12800. Wells were incubated 2 hours at room temperature, then were washed and a 1/5000 dilution of anti Guinea Pig IgG HRP in 0.1% BSA in PBS Tween was prepared and then 100 μl added to the washed well. This incubated for 1 hour and was washed. OPD substrate was prepared using Thermo-Fisher OPD tablets at 1 tablet per 10 mls. Thermo fisher substrate buffer was added at 1/10 and each well had 100 μl added and incubated for 15 minutes. 50 μl of 2N H₂SO₄ was added to stop the reaction and plates were read on a molecular devices spectromax at 490 nM. Titer defined as the dilution giving 50% maximum OD and was extrapolated if it fell between dilutions

### Titer Guinea Pig Bleeds on Tau

2 μg/ml recombinant WT Tau 4R2N was coated on to the plate using 100 μl per well in PBS and incubated overnight at room temperature. Plates were blocked for 1 hour with 1% BSA in PBS. Plates were aspirated and to row A 200 μl of 0.1% BSA in PBS Tween was added. In column 1 neg. GP serum was added at 1/100 while the rest of the row contained 1/100 test serums. Rows were serially diluted ½ down the plate giving dilution of 1/100 to 1/12800. Wells incubated 2 hours at room temperature then were washed and a 1/5000 dilution of anti-Guinea Pig IgG HRP in 0.1% BSA in PBS Tween was prepared and then 100 μl added to the washed well. This incubated for 1 hour and was washed. OPD substrate was prepared using Thermo-Fisher OPD tablets at 1 tablet per 10 mls. Thermo fisher substrate buffer was added at 1/10 and each well had 100 μl added and was incubated for 15 minutes. 50 μl of 2N H₂SO₄ was added to stop the reaction and plates were read on a molecular devices spectromax at 490 nM. Titer was defined as the dilution giving 50% maximum OD and was extrapolated if it fell between dilutions.

### Titer Guinea Pig Bleed on Aβ 1-28 or Aβ 1-15

Aβ 1-15 and Aβ 1-28 were both used at different parts of the study. Both of these will not form aggregates. 2 μg/ml Aβ monomers were coated at coated on to the plate 100 μl per well in PBS and incubated overnight at room temperature. Plates were blocked for 1 hour with 1% BSA in PBS. Plates were aspirated and to row A 200 μl of 0.1% BSA in PBS Tween was added. In column 1 neg. GP serum was added at 1/100 while the rest of the row contained 1/100 test serums. Rows were serially diluted ½ down the plate giving dilution of 1/100 to 1/12800. Wells incubated 2 hours at room temperature then were washed and a 1/5000 dilution of anti Guinea Pig IgG HRP in 0.1% BSA in PBS Tween was prepared and then 100 μl added to the washed well. This incubated for 1 hour and was washed. OPD substrate was prepared using Thermo-Fisher OPD tablets at 1 tablet per 10 mls. Thermo fisher substrate buffer was added at 1/10 and each well had 100 μl added and was incubated for 15 minutes. 50 μl of 2N H₂SO₄ was added to stop the reaction and plates were read on a molecular devices spectromax at 490 nM. Titer was defined as the dilution giving 50% maximum OD and was extrapolated if it fell between dilutions.

### Titering the Tau Against a Peptide Containing its Epitope

Thermofisher neutavidin plates were rehydrated with 0.05% Tween in TBS and aspirated. Peptide GGGSVQIVYKPVDLS (SEQ ID NO:68) containing a biotin was made up in a 1/500 dilution in 0.1% BSA in PBS tween. Adding 100 μl per well for 1 hour and then washed. To row A of the plate 200 μl of 0.1% BSA in PBS Tween was added. In column 1 neg. GP serum was added at 1/100 while the rest of the rows contained 1/100 test serums. Rows were serially diluted ½ down the plate giving dilution of 1/100 to 1/12800. Wells incubated 2 hours at room temperature then were washed and a 1/5000 dilution of anti Guinea Pig IgG HRP in 0.1% BSA in PBS Tween was prepared and then 100 μl added to the washed well. This incubated for 1 hour and was washed. OPD substrate was prepared using Thermo-Fisher OPD tablets at 1 tablet per 10 mls. Thermo fisher substrate buffer was added at 1/10 and each well had 100 μl added and was incubated for 15 minutes. 50 μl of 2N H₂SO₄ was added to stop the reaction and plates were read on a molecular devices spectromax at 490 nM. Titer defined as the dilution giving 50% maximum OD and was extrapolated if it fell between dilutions.

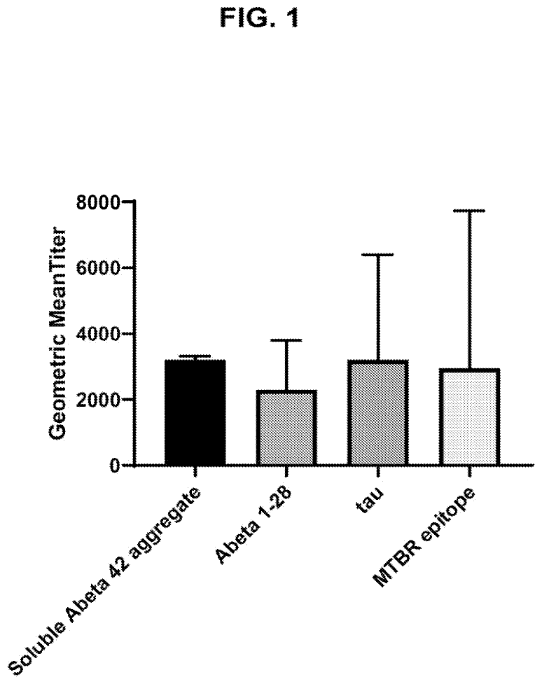

FIG. 1 shows the results comparing the geometric mean titers of Guinea Pig serum for immunogen DAEFRHDRRQIVYKPV (SEQ ID NO:57) titered on monomeric Aβ 1-28, soluble aggregates of Aβ, full length tau, and tau MTBR epitope containing

(SEQ ID NO: 68)
GGGSVQIVYKPVDLS.

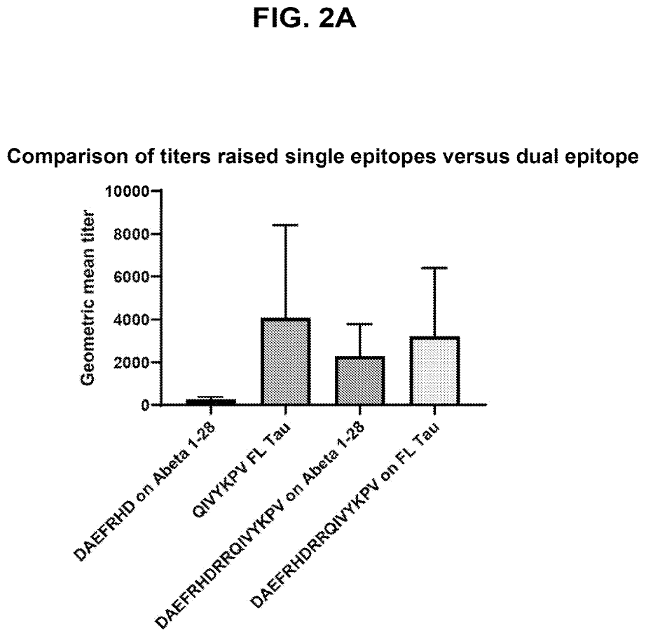

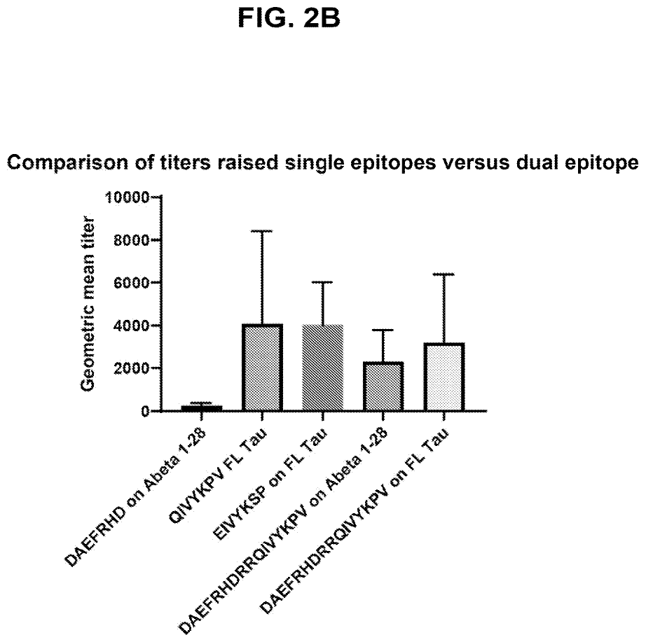

FIG. 2 shows the results for a single peptide immunogen Aβ and tau peptides (DAEFRHD (SEQ ID NO:06) and QIVYKPV (SEQ ID NO:39)) versus dual peptide immunogen DAEFRHDRRQIVYKPV (SEQ ID NO:57) on Aβ 1-28 and full-length tau.

### Example 3: Staining of Alzheimer's Brain Tissue with Sera from Guinea Pigs Immunized with a Vaccine as Disclosed Herein

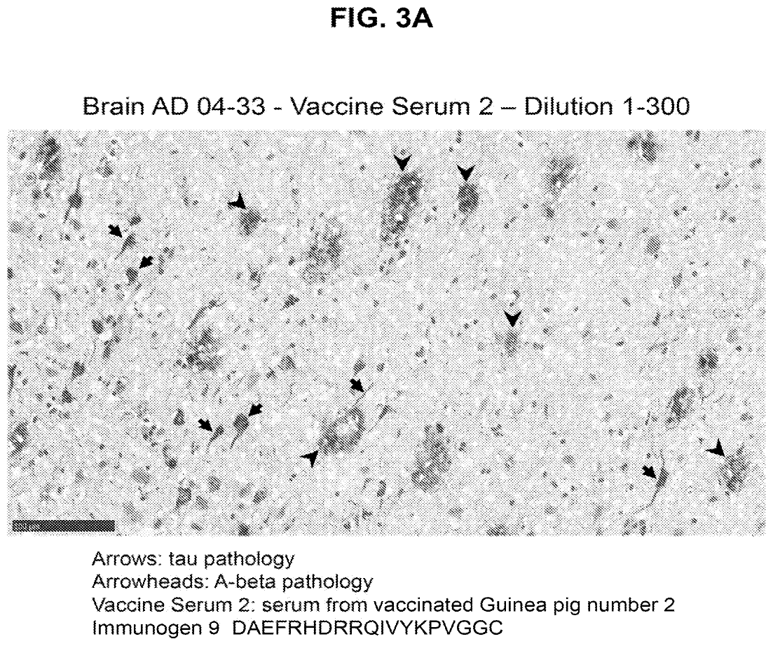

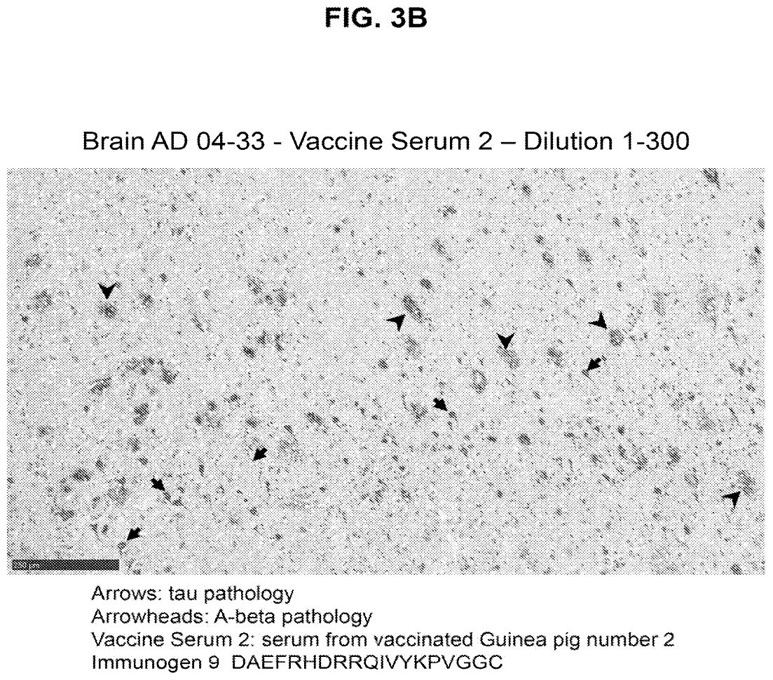

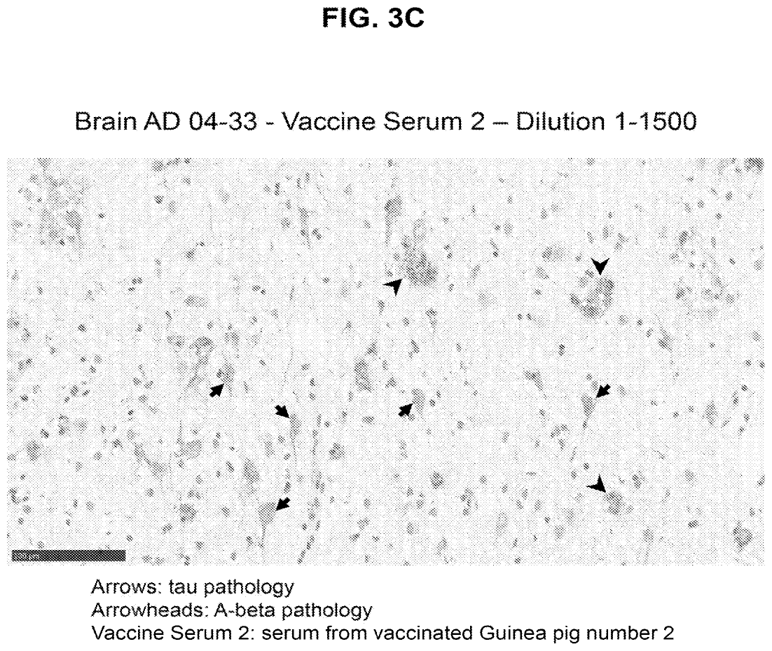

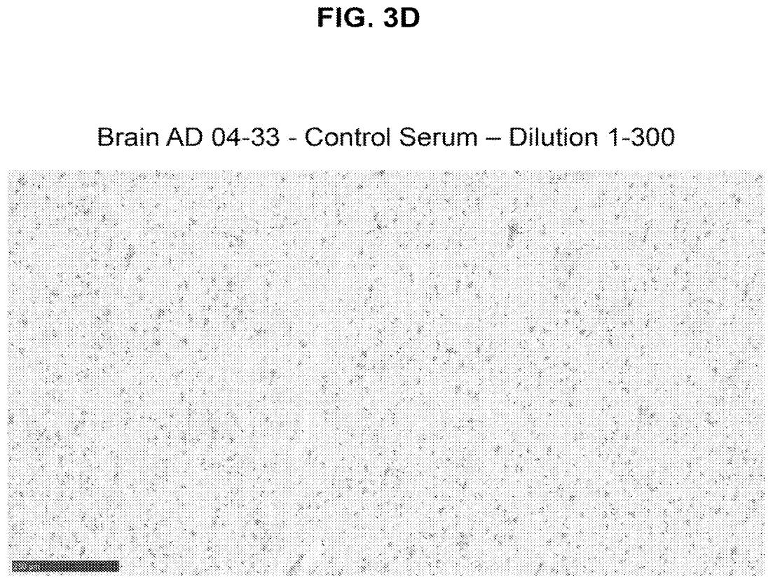

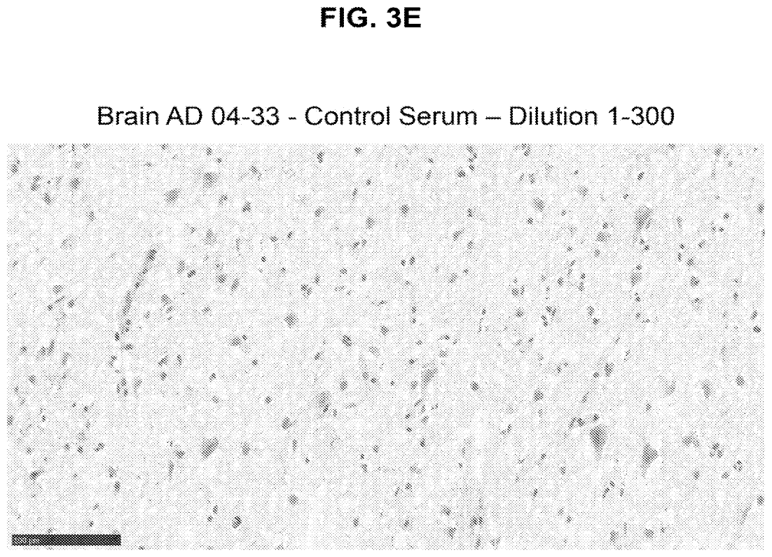

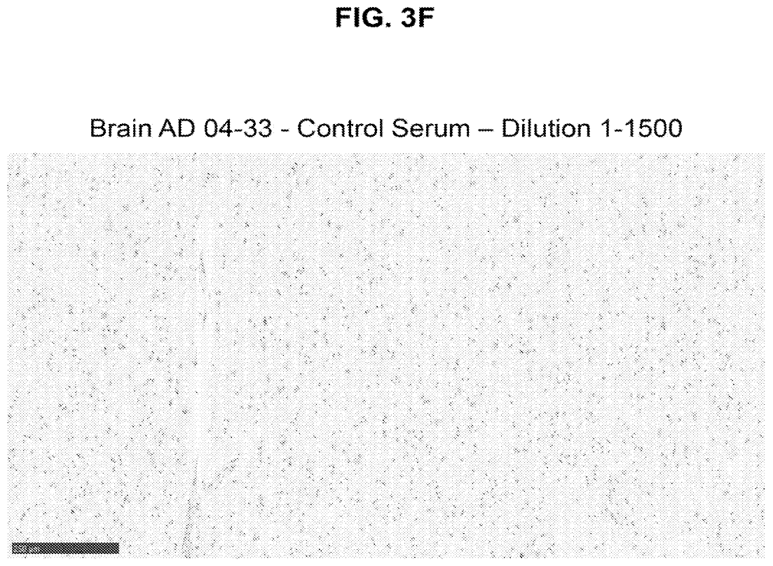

Autopsy blocks of fresh frozen human brain tissue (˜ 0.5 g) were embedded in optimal cutting temperature compound (OCT compound) and cut using a cryostat to generate 10 μm sections. The sections were placed into a solution of glucose oxidase and beta D-glucose, in the presence of sodium azide, to block endogenous peroxidase. Once tissue sections were prepared, the staining with the specified Guinea pig sera from Guinea pigs immunized with a vaccine as disclosed herein was carried out at two dilutions (1:300 and 1:1500), using a rabbit anti-guinea pig secondary antibody and a DAKO DAB Detection Kit as per the manufacturer's instructions. The staining was processed using an automated Leica Bond Stainer. The results indicate that sera from Guinea pigs immunized with a vaccine as disclosed herein comprises antibodies specific to Aβ and tau in human brain tissue of Alzheimer's patients (see FIG. 3A-3F)

### Example 4: Serum from Vaccinated Animals Blocks Soluble Aβ Aggregates from Binding to Neurons

E18 primary rat hippocampal neurons were cultured as described previously (Zago, et al. “Neutralization of Soluble, Synaptotoxic Amyloid β Species by Antibodies Is Epitope Specific,” J Neurosci. 2012 Feb. 22; 32(8): 2696-2702). Soluble Aβ aggregate was pre-incubated with or without guinea pig vaccine serum on culture DIV14-21 to block soluble Aβ aggregate from neuritic binding. Guinea pig serum was isolated from animals vaccinated with dual immunogen peptide: DAEFRHDRRQIVYKPVGGC (SEQ ID NO:59; Immunogen 9). Fresh unlabeled, biotinylated or (9:1) soluble Aβ was prepared one day prior and incubated overnight at 4° C. Each diluted serum sample (1:1000, 1:300, and 1:100) and soluble Aβ solution was prepared at 2× the final concentration in one-half of final treatment volume using NeuroBasal-no phenol red (NB-NPR) medium. This was combined with one-half final volume of 2× soluble Aβ and with one-half final volume of 2× diluted guinea pig vaccine serum to make up a 1× final concentration in total final treatment volume, which was mixed well and then pre-incubated for 30 minutes at 37° C. E18 neurons were rinsed with NB-NPR at 150 μL/well before adding binding treatment. Guinea pig serum from vaccinated animals/Aβ treatment was added to E18 neurons at 60 μL/well, and then incubated for 30 minutes at 37° C. under normal incubator conditions (5% CO₂; 9% (2). Cells were rinsed twice using 150 μL/well of NB-NPR, and then fixed in 4% paraformaldehyde in 1×DPBS for 20 minutes. Cells were permeabilized using 0.1 TX-100 for 5 minutes, and blocked using 10% normal goat serum (NGS) for 1 hour at room temperature (RT). Cells were incubated with MAP2 & NeuN primary antibodies in 100 μL/well, 1×DPBS containing 1% BSA+1% NGS overnight at 4° C. The next day, cells were rinsed twice in 150 μL/well 1×DPBS for 5 minutes each wash. Secondary antibodies were added for 1 hour at RT in 100 μL/well 1×DPBS+1% BSA+1% NGS. High-content imaging (HCl) analysis was performed to quantify soluble aggregate Aβ neuritic binding using Operetta HCl CLS instrument (Perkin Elmer; modified Neurite Outgrowth algorithm: 40×H₂O objective; 40 fields per well; (n=3) per condition; data shown as mean (+/−) SD); MAP2 & NeuN (Abcam) neuronal markers used to each trace neurite tree and count cell body number per optical field; Neuritic Aβ soluble aggregate spots detected using streptavidin-488 or polyclonal Aβ antibody (Thermo; Millipore); and data reported as Aβ soluble aggregate spots/neuron (or as Integrated Intensity)).

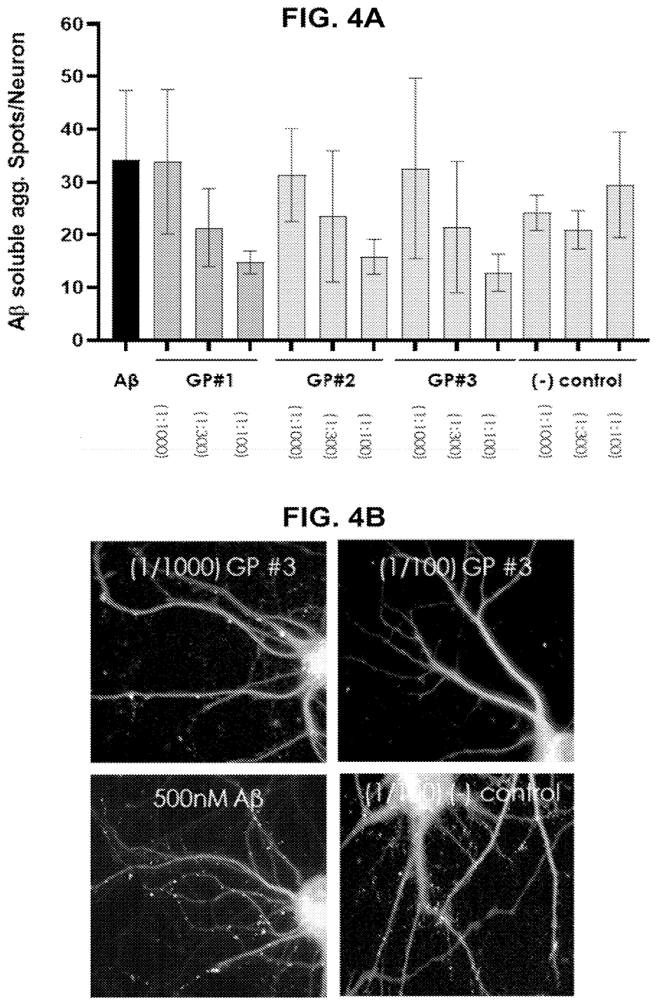

Approximately 80-150 neurons were observed per well for each condition tested. The results demonstrate that Guinea pig serum from animals vaccinated with a dual immunogen peptide, DAEFRHDRRQIVYKPVGGC (SEQ ID NO:59; Immunogen 9), inhibited Abeta binding to neurons in a dose dependent manner (see FIG. 4).

### Example 5: Mice Vaccinated with Dual Peptide Antigens Produce Titers to Aβ and Tau

Swiss Webster Female mice were injected on day 0, 14 and 28 with 25 μg of a dual peptide immunogen (Table 2) and 25 μg QS21 (Desert King) in PBS total 200 μl/injection. Each mouse received 200 μl subcutaneously. Mice were bled on day 21 and 35.

TABLE 2

| | Dual peptide immunogen sequence |
|---|---|
| Immunogen | (SEQ ID NO.) |
| 9 | DAEFRHDRRQIVYKPVGGC (SEQ ID NO: 59) |
| 10 | DAEFRHDRRQIVYKPVC (SEQ ID NO: 60) |
| 18 | DAEFRHDRRQIVYKPVAAC (SEQ ID NO: 61) |
| 19 | DAEFRHDRRQIVYKPVKKC (SEQ ID NO: 62) |
| 20 | DAEFRHDRREIVYKSPGGC (SEQ ID NO: 63) |
| 21 | DAEFRHDRREIVYKSPAAC (SEQ ID NO: 64) |
| 22 | DAEFRHDRREIVYKSPC (SEQ ID NO: 65) |
| 23 | DAEFRHDRREIVYKSPKKC (SEQ ID NO: 66) |

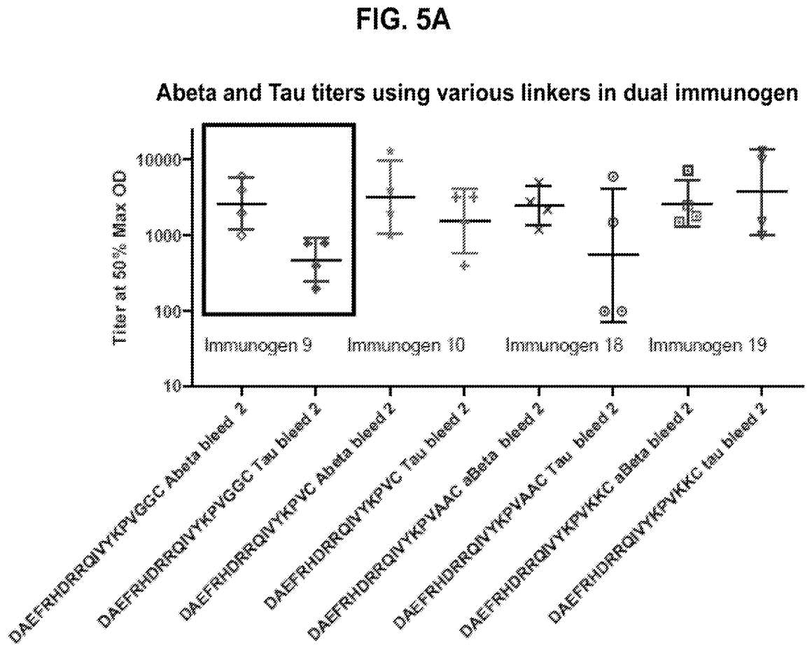

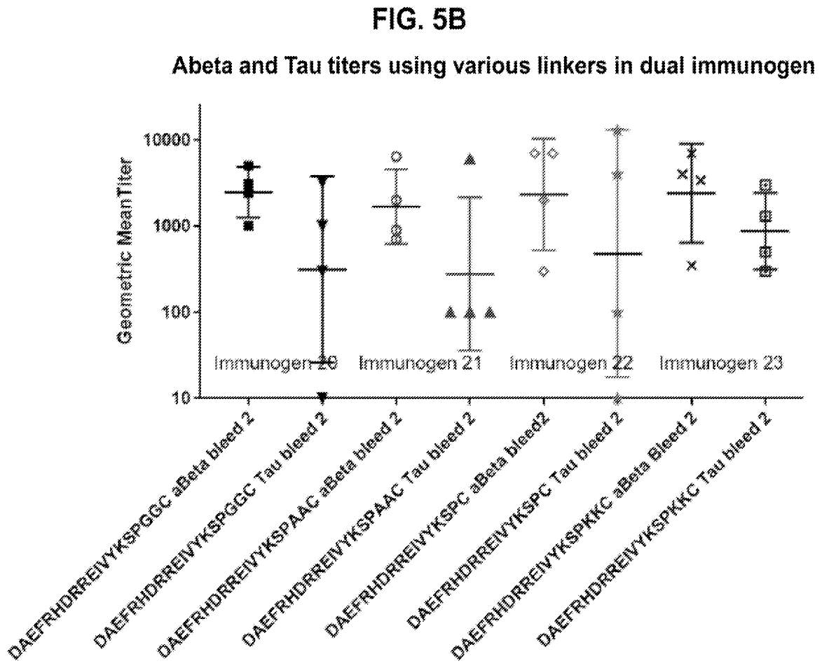

The immunogens containing Tau peptide EIVYKSP (SEQ ID No:43); see FIG. 5B) showed greater variability overall in tau titers (with the exception of immunogen 23) than those with the tau MTBR sequence QIVYKPV (SEQ ID NO:39); therefore the base microtubule binding region (MTBR) peptide containing DAEFRHDRRQIVYKPV (SEQ ID NO:57) was also tested (FIG. 5A). Titers of three immunogens with different linkers (no linker, AA and KK) compared to the GGC linker are shown in FIG. 5.

Titers observed from the second bleed are set forth in Tables 3 and 4:

TABLE 3
Titer Against Abeta 1-28
(at 50% Max OD)

| | | | | | | geometric |
|---|---|---|---|---|---|---|
| Immunogen | mouse # | | | | geometric | mean SD |
| number | 1 | 2 | 3 | 4 | mean | factor |
| 9 | 4000 | 2000 | 6000 | 1000 | 2632.15 | 2.20 |
| 10 | 13000 | 2000 | 4000 | 1000 | 3193.44 | 2.98 |
| 18 | 2000 | 2800 | 1200 | 5000 | 2407.6 | 1.80 |
| 19 | 7200 | 2500 | 1500 | 1800 | 2640.34 | 2.02 |
| 20 | 4000 | 2000 | 6000 | 1000 | 2470 | 1.96 |
| 21 | 13000 | 2000 | 4000 | 1000 | 1665 | 2.71 |
| 22 | 300 | 7000 | 2000 | 7000 | 2329 | 4.43 |
| 23 | 350 | 3400 | 4000 | 7000 | 2403 | 3.74 |

TABLE 4
Titer Against Tau
(at 50% Max OD)

| | | | | | | geometric |
|---|---|---|---|---|---|---|
| Immunogen | mouse # | | | | geometric | mean SD |
| number | 1 | 2 | 3 | 4 | mean | factor |
| 9 | 400 | 800 | 200 | 800 | 475.7 | 1.94 |
| 10 | 400 | 3200 | 3200 | 1500 | 1574.4 | 2.67 |
| 18 | 1500 | 100 | 6000 | 100 | 547.7 | 7.72 |
| 19 | 10000 | 1000 | 13000 | 1500 | 3738.9 | 3.68 |
| 20 | 300 | 3200 | No titer | 1000 | 313 | 12.07 |
| 21 | 1500 | 100 | 6000 | 100 | 278.3 | 7.75 |
| 22 | 100 | 4000 | No titer | 13000 | 477.5 | 27.33 |
| 23 | 300 | 3000 | 1300 | 500 | 874.8 | 2.78 |

Titers observed in guinea pigs immunized with Immunogen 9 DAEFRHDRRQIVYKPVGGC (SEQ ID NO:59) are set forth in Table 5 below.

TABLE 5

| | | Titer Against | | |
|---|---|---|---|---|
| | Titer | Abeta | Titer | Titer Against bio- |
| Guinea | Against | soluble | Against | GGSQIVYKPVDLS |
| Pig | Abeta 1-28 | aggregate | Tau | (SEQ ID NO: 73) |
| 1 | 3100 | 1500 | 1600 | 1000 |
| 2 | 3200 | 4000 | 6400 | 6400 |
| 3 | 3300 | 2000 | 3200 | 4000 |

### Example 6: Serum from Vaccinated Animals Stains Aβ Plaques and Tau Pathologies in Human Brain Tissue

Fresh frozen human brain tissues from autopsied Alzheimer's disease donors or non-diseased controls was embedded in OCT, and cut in a cryostat to generate 10 μm frozen sections. The tissue sections were incubated in a solution of glucose oxidase and beta D-glucose, in the presence of sodium azide, to block endogenous peroxidase. The staining with sera from vaccinated mice. Mice were vaccinated with the following dual antigen peptides: DAEFRHDRRQIVYKPVGGC (SEQ ID NO:59, Immunogen 9); DAEFRHDRRQIVYKPVC (SEQ ID NO:60, Immunogen 10); DAEFRHDRRQIVYKPVAAC (SEQ ID NO:61, Immunogen 18); and DAEFRHDRRQIVYKPVKKC (SEQ ID NO:62, Immunogen 19) or control mice. Staining was then carried out at 1:1000 dilution, in an automated Leica Bond Rx Stainer (Leica Biosystems). Antibody binding was detected using the Bond Polymer Refine Detection Kit (DS9800, Leica Biosystems), which is based on an anti-mouse polymer detection, DAB visualization and hematoxylin nuclear counter-staining. After cover-slipping, the stained tissue slides were digitally imaged with a Hamamatsu NanoZoomer 2.0HT slide scanner (Hamamatsu Corporation) with an NDP.scan, 2.5.85 software. The digitized images were viewed and analyzed using the NDP.view, 2.7.43.0 software.

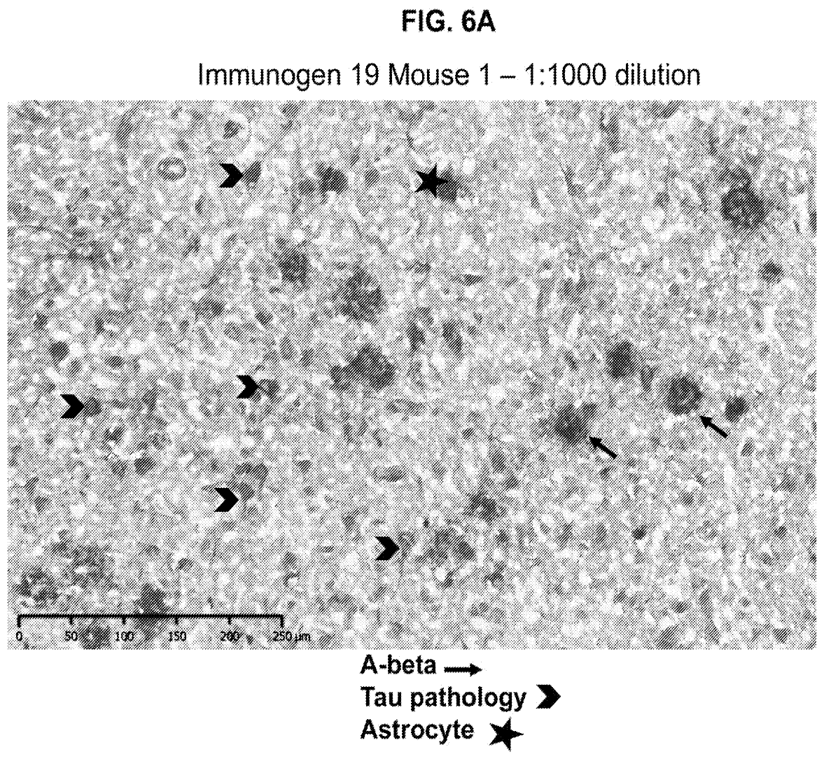

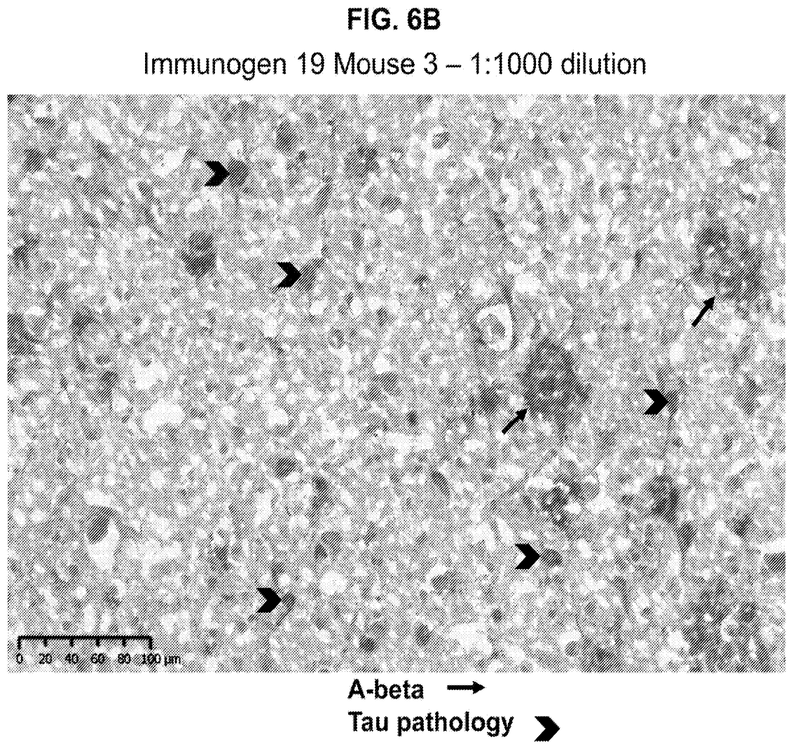

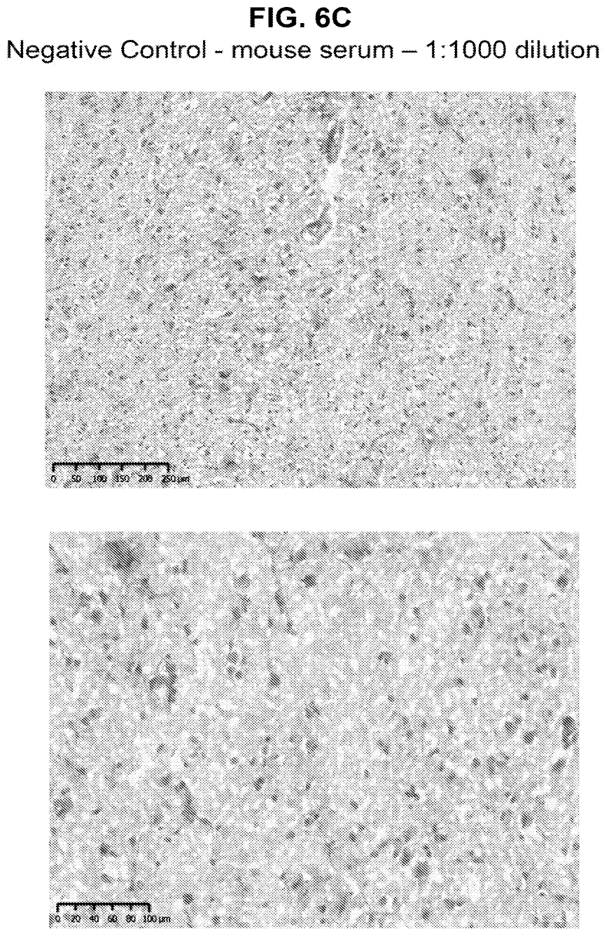

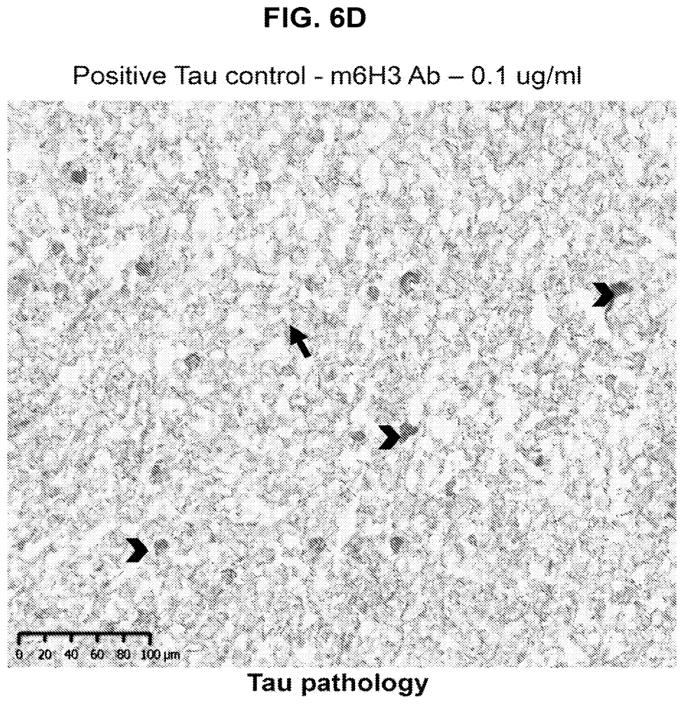

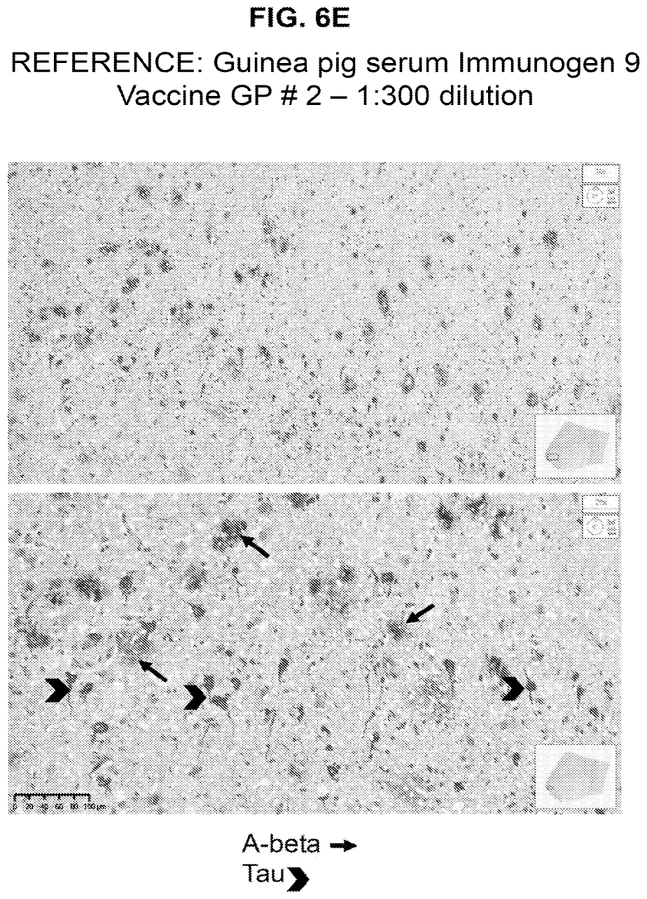

Results demonstrate Aβ plaques and tau neurofibrillary tangles were identified based on their typical histopathological characteristics. Such pathologies were absent from tissues incubated with control mouse serum. Also, non-diseased tissue had no such pathological staining after incubation with the sera from vaccinated mice. FIGS. 6A-E and Table 6 summarize the results of Aβ and Tau staining using mouse sera from animals vaccinated with dual antigen peptides (1:1000 dilution of sera used to stain human brain tissue from AD patients).

TABLE 6

| | Aβ | Tau | |
|---|---|---|---|
| Serum ID | staining | pathology | Background |
| 9-1 | + | | Clear |
| 9-2 | + | | Dark |
| 9-3 | +++++ | | Clear |
| 9-4 | ++ | | Clear |
| 10-1 | +++++ | | OK |
| 10-2 | +++ | | Clear |
| 10-3 | ++++ | * | OK |
| 10-4 | ++++ | * | Dark |
| 18-1 | ++ | | Dark |
| 18-3 | + | | Clear |
| 19-1 | +++++ | +++ | OK |
| 19-2 | ++ | * | OK |
| 19-3 | + | ++ | Dark |
| 19-4 | ++ | | OK |

9: DAEFRHDRRQIVYKPVGGC (SEQ ID NO: 59)
10: DAEFRHDRRQIVYKPVC (SEQ ID NO: 60)
18: DAEFRHDRRQIVYKPVAAC (SEQ ID NO: 61)
19: DAEFRHDRRQIVYKPVKKC (SEQ ID NO: 62)
* possible tau pathology, too weak to clearly identify staining as tau

### Example 7: Immunization and Determination of Titers in Cynomolgus Monkeys

The study described in this example was designed to assess the development of titers to Aβ and tau in cynomolgus monkeys using the dual antigen peptide DAEFRHDRRQIVYKPVGGC (SEQ ID NO:59). We also assessed two injection schedules and persistence of titers.

### Immunizations

Several dual Aβ-tau linear immunogens with a dendritic cell cleavage site were screened in mice for balanced titer on Aβ and tau proteins (see Example 5, above). A subset of immunogens were further evaluated in guinea-pig (see Examples 1-5, above) and cynomolgus monkey (this example). Two groups of four monkeys were immunized intramuscularly with 50 μg of immunogen (SEQ ID NO:59; DAEFRHDRRQIVYKPVGGC) coupled to CRM197 carrier protein, and 50 μg of the adjuvant QS21. Group 1 was injected on week 0, 4, 12 and 24 with bleeds taken every 2 weeks through week 38. Group 2 was injected at week 0, 8, 24 with bleeds taken every 2 weeks through week 38. Serum titer levels were determined against Aβ and full-length tau. Fresh frozen human AD or control brain sections were stained with sera from immunized and control animals. The activity of the guinea-pig immune sera was also assessed on soluble Aβ oligomer binding in primary rat hippocampal neurons.

### Titer Protocol

### Measurement of Antibody Titer to Aβ 1-28 in Cynomolgus Monkey

Cynomolgus monkey bleeds were titered by enzyme-linked immunosorbent assay (ELISA). Plates were coated overnight at 2 μg/mL with Aβ 1-28 (SEQ ID NO:67) in phosphate-buffered saline (PBS) and then blocked 1 hour with 1% bovine serum albumin (BSA) in PBS. Pre-bleed cynomolgus monkey was used as a negative control while known positive anti-serum from previous mouse studies was used as a positive control at the same dilutions of test serum. Bleeds were diluted in PBS/0.1% BSA/0/1% Tween 20 (PBS/BSA/T) starting at 1/100 and serially diluted 1:2 down the plate. Plates were washed with TBS/Tween 20 and goat anti-monkey immunoglobulin G (IgG) (heavy+light chains) horseradish peroxidase (HRP) (IgG [H+L] HRP; Invitrogen) was added and incubated 1 hour at room temperature. Plates were washed in TBS/Tween 20, and antibody binding was detected with o-phenylenediamine dihydrochloride (OPD) substrate (Thermo Fisher Scientific, Waltham, MA) following manufacturer's instructions. Plates were read at 490 nM on a Molecular Devices Spectromax. Titer was defined as the dilution giving 50% Maximum OD or 4× background (defined in graphs and tables) extrapolation was used if it fell in between dilutions.

### Measurement of Antibody Titer to Tau in Cynomolgus Monkey

Cynomolgus monkey bleeds were titered by enzyme-linked immunosorbent assay (ELISA) against full-length recombinant tau (Proteos, Kalamazoo, MI; SEQ ID NO:02). Plates were coated overnight at 2 μg/mL tau in phosphate-buffered saline (PBS) and then blocked 1 hour with 1% bovine serum albumin (BSA) in PBS. Pre-bleed guinea pig serum was used as a negative control while known positive anti-serum from previous mouse studies was used as a positive control at the same dilutions of test serum. Bleeds were diluted in PBS/0.1% BSA/0/1% Tween 20 (PBS/BSA/T) starting at 1/100 and serially diluted 1:2 down the plate. Plates were washed with TBS/Tween 20 and goat anti-monkey immunoglobulin G (IgG) (heavy+light chains) horseradish peroxidase (HRP) (IgG [H+L] HRP; Invitrogen) was added and incubated 1 hour at room temperature. Plates were washed in TBS/Tween 20, and antibody binding was detected with o-phenylenediamine dihydrochloride (OPD) substrate (Thermo Fisher Scientific, Waltham, MA) following manufacturer's instructions. Plates were read at 490 nM on a Molecular Devices Spectromax. Titer was defined as the dilution giving 50% Maximum OD or 4× background (defined in graphs and tables) extrapolation was used if it fell in between dilutions.

### Titer Cynomolgus Monkey Bleeds on the Carrier Protein CRM197

Cynomolgus monkey bleeds were titered by enzyme-linked immunosorbent assay (ELISA) against CRM197 (FinaBio, Maryland) carrier protein. Plates were coated overnight at 2 μg/mL tau in phosphate-buffered saline (PBS) and then blocked 1 hour with 1% bovine serum albumin (BSA) in PBS. Pre-bleed guinea pig serum was used as a negative control while known positive anti-serum from previous mouse studies was used as a positive control at the same dilutions of test serum. Bleeds were diluted in PBS/0.1% BSA/0/1% Tween 20 (PBS/BSA/T) starting at 1/100 and serially diluted 1:2 down the plate. Plates were washed with TBS/Tween 20 and goat anti-monkey immunoglobulin G (IgG) (heavy+light chains) horseradish peroxidase (HRP) (IgG [H+L] HRP; Invitrogen) was added and incubated 1 hour at room temperature. Plates were washed in TBS/Tween 20, and antibody binding was detected with o-phenylenediamine dihydrochloride (OPD) substrate (Thermo Fisher Scientific, Waltham, MA) following manufacturer's instructions. Plates were read at 490 nM on a Molecular Devices Spectromax. Titer was defined as the dilution giving 50% Maximum OD or 4× background (defined in graphs and tables) extrapolation was used if it fell in between dilutions.

### Results

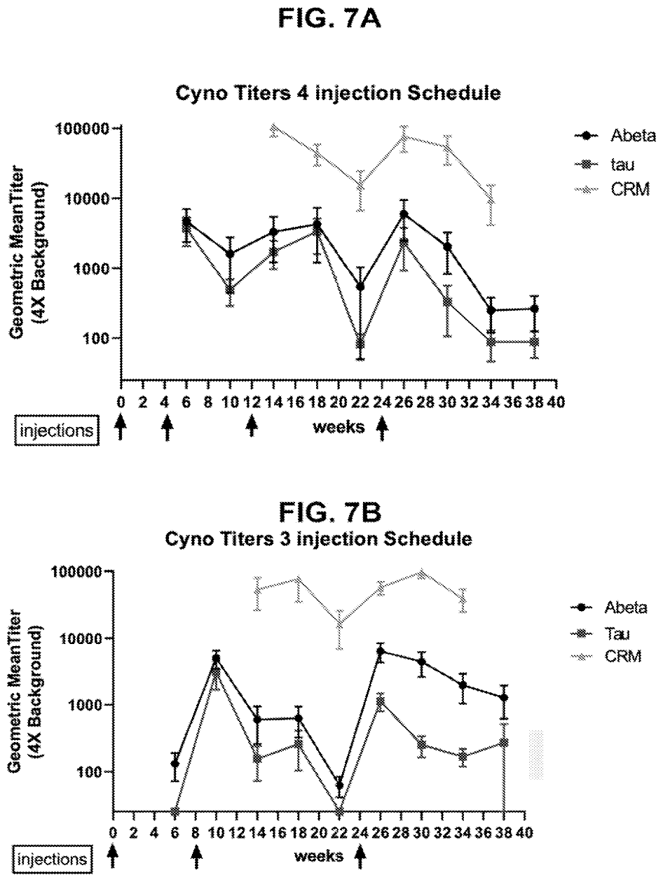

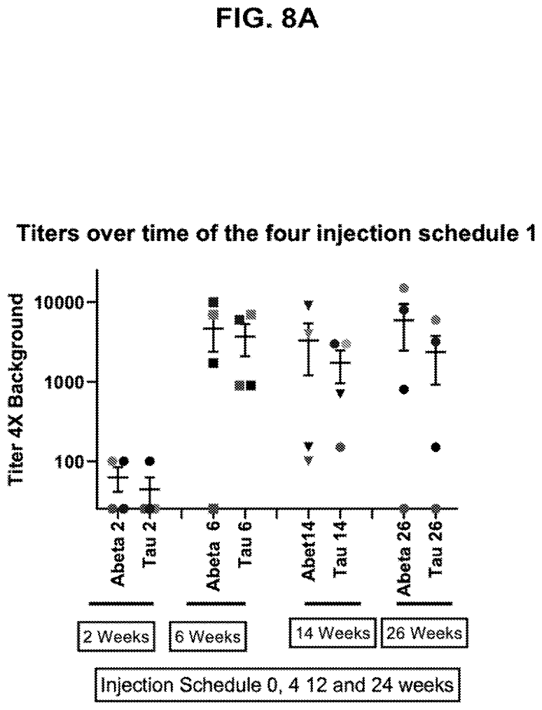

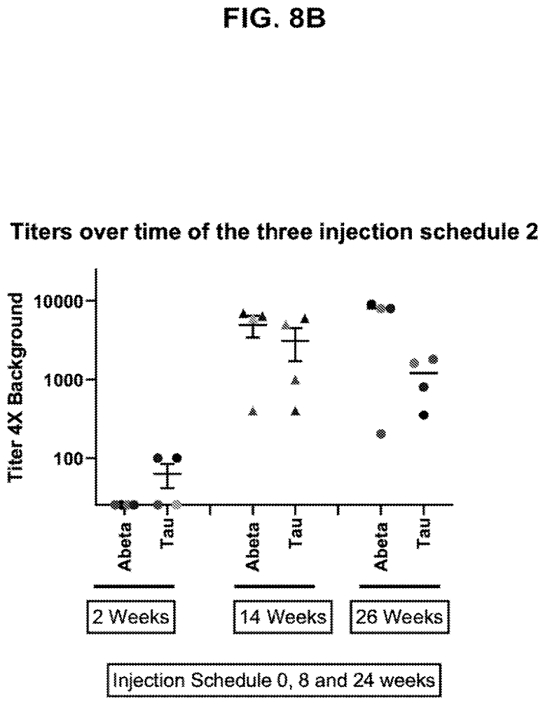

Titers levels were similar for Aβ and tau. Monkeys subject to the group 1 vaccination schedule had titers with a greater area under the curve than those of group 2 but did not show a significant improvement in maximum titer under these conditions (FIG. 7A, FIG. 7B). Overall titer levels were lower than the guinea pig study described above. This change may be attributable to a change of adjuvant from QS21 in ADDVAX in the Guinea pig to QS21 in phosphate buffered saline (PBS) in cynomolgus monkeys to match approved vaccines. Persistence of titer was modest for both four injection and three injection vaccination schedules (FIG. 8A, FIG. 8B). These results demonstrate that these immunogens can generate a balanced immune response to Aβ and tau in primates. Optimization of dosing schedule and/or formulation may improve titer levels and persistence.

### Example 8: Immunohistochemistry on AD and Control Brain Sections

Sera from immunized monkeys were evaluated for the ability to bind pathological Aβ plaques and tau tangles in human brain tissue from subjects with AD. Binding both Aβ plaques and tau tangles is expected reduce plaque burden and reduce tau transmission which in turn should reduce the signs and symptoms of AD.

Autopsy blocks of fresh frozen human brain tissue were embedded in optimal cutting temperature compound (OCT compound) and cut using a cryostat to generate 10 micron sections. The sections were placed into a solution of glucose oxidase and beta D-glucose, in the presence of sodium azide, to block endogenous peroxidase. Once tissue sections were prepared, the staining with the specified cynomolgus immune sera was carried out at two dilutions (1:300 in 5% goat serum with 0.25% Triton for 1 hour at RT). Binding was detected with a mouse anti-monkey IgG secondary antibody purified unlabeled (Mybiosource 3 mg/mL) for 1 hour at RT, and a goat anti-mouse IgG secondary antibody (Jackson, 1:200) for 1 hour at RT, avidin-biotin complex (ABC; Vector PK-4000) and a DAKO DAB Detection Kit according to manufacturer instructions. The staining was processed using an automated Leica Bond Stainer.

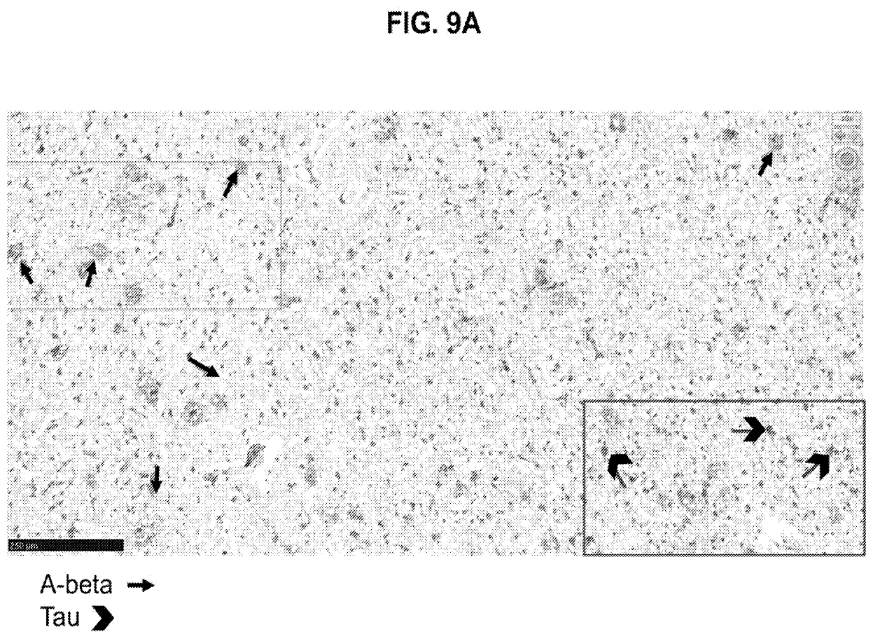

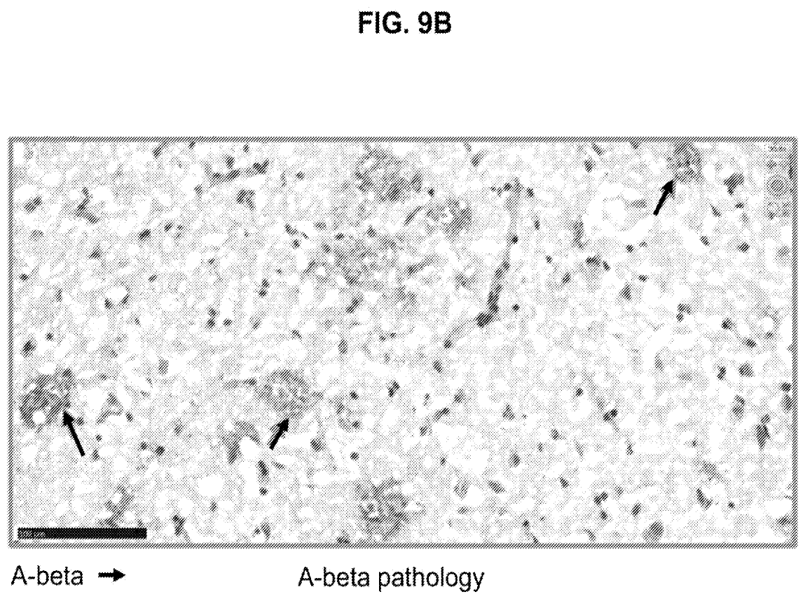

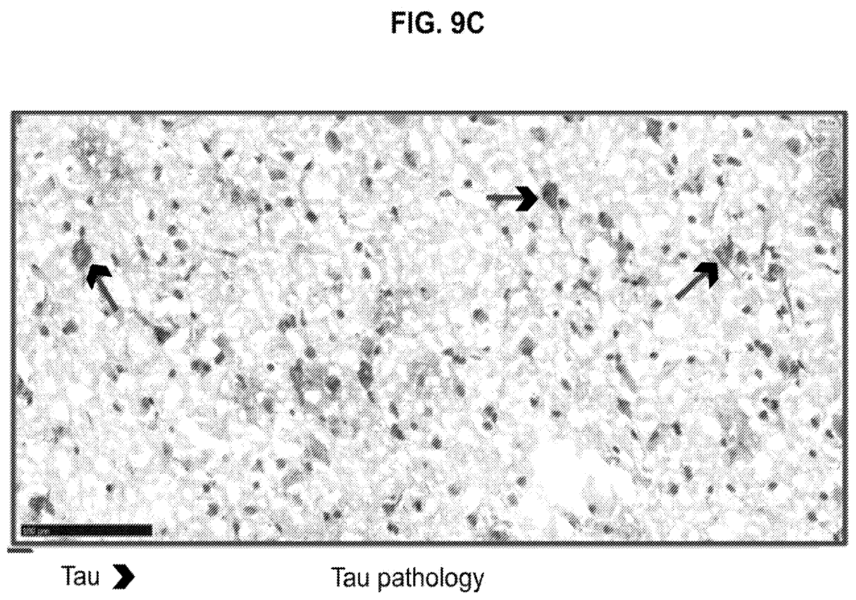

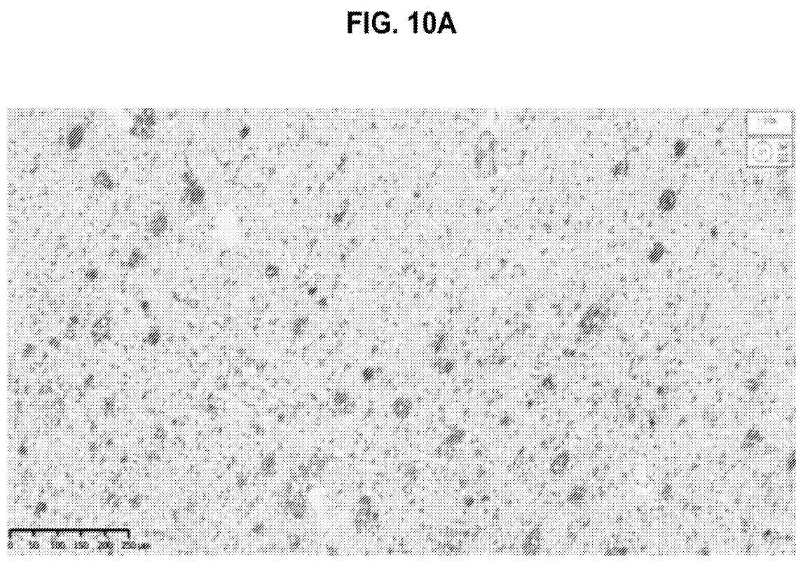

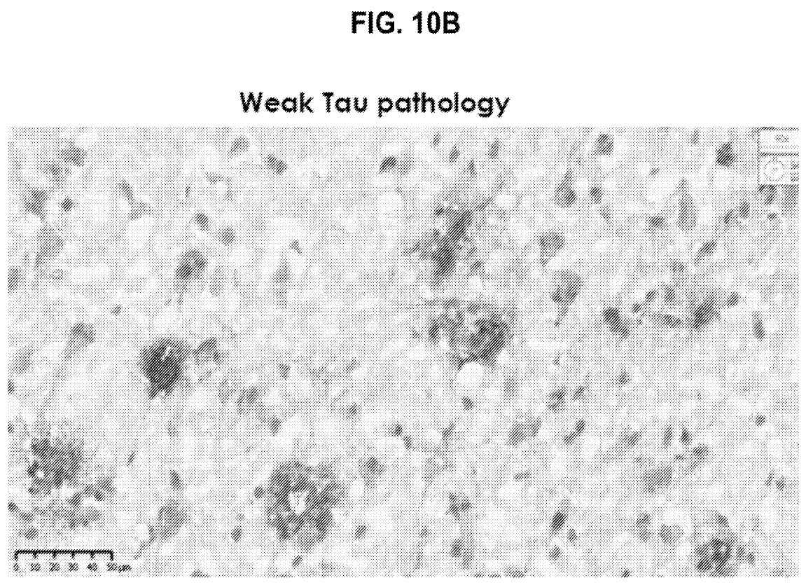

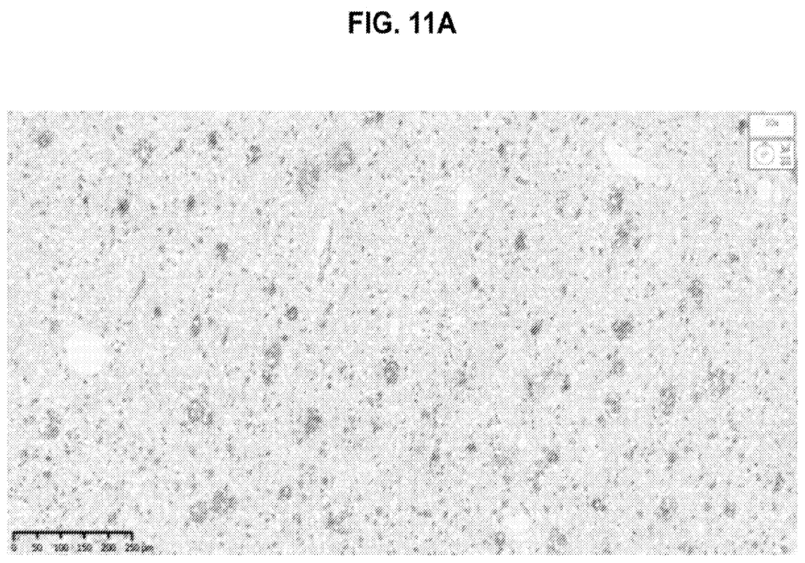

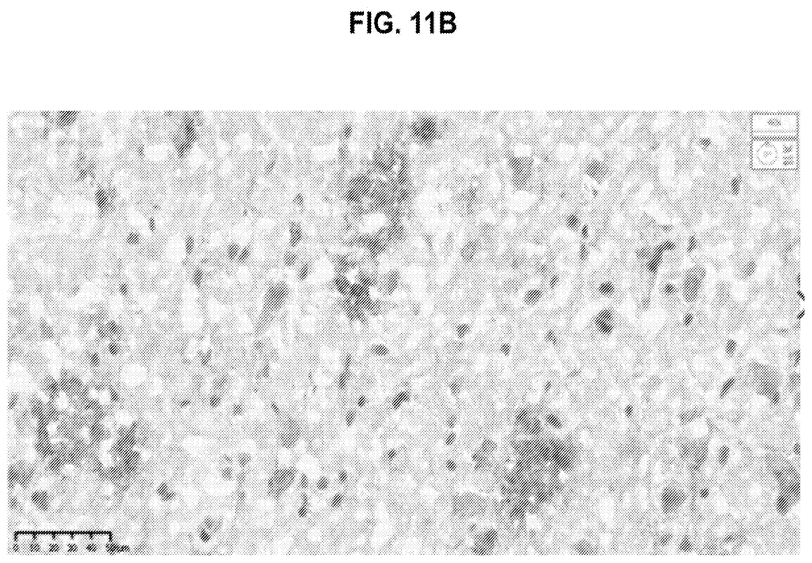

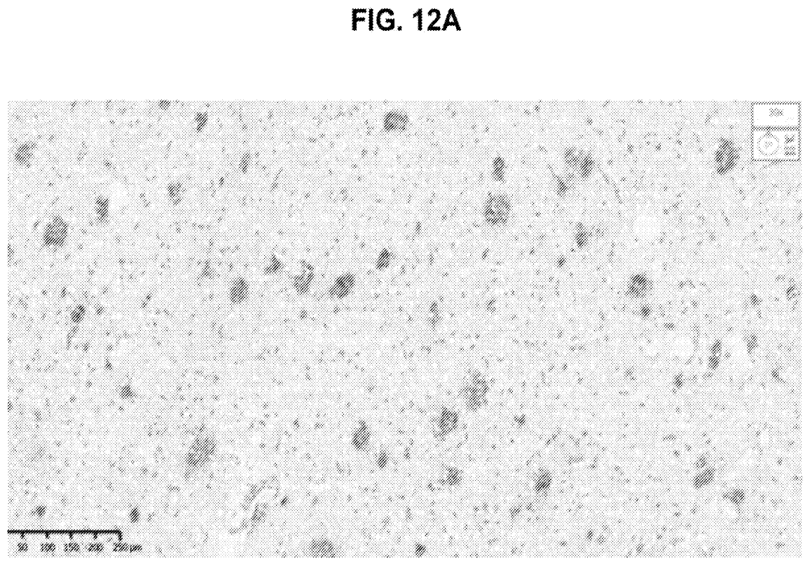

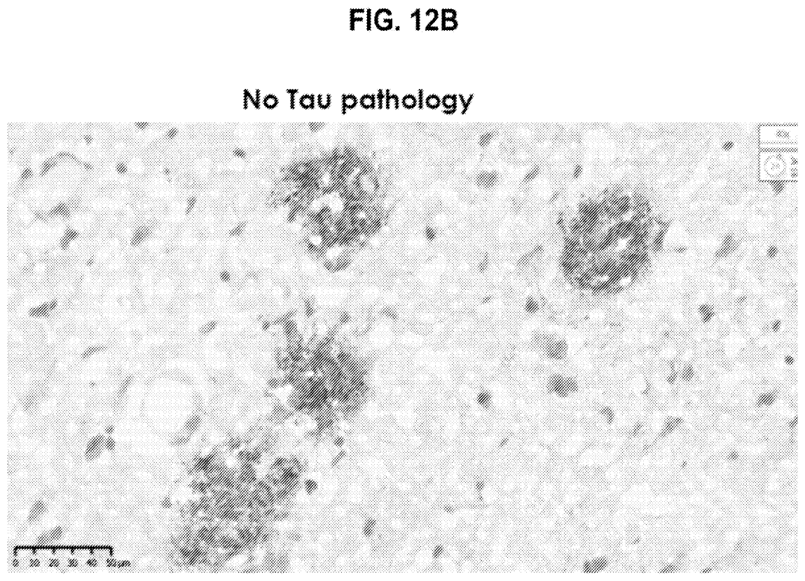

Table 7 shows a summary of the staining in the cynomolgus monkey study. All animal except 1002 had positive staining for Aβ. Only animal 1001 had strong tau staining (FIG. 9A-FIG. 9C), with 1003 (FIG. 10A-FIG. 10B) and 1501 (FIG. 11A-FIG. 11B) demonstrating modest staining. Animal 2501, for example, did not demonstrate significant tau staining (FIG. 12A-FIG. 12B). There did not appear to be a correlation with tau staining and tau titers as evidenced by animal 1001, which did not have a strong tau titer but stained tau strongly.

TABLE 7

| Animal | Aβ | Tau | Aβ | Tau |
|---|---|---|---|---|
| Number | Staining | Staining | Titer | Titer |
| 1001 | + | ++ | 800 | 150 |
| 1002 | − | − | 25 | 25 |
| 1003 | +++ | + | 15000 | 6000 |
| 1501 | +++ | +/− | 8000 | 3200 |
| 2001 | +++ | − | 9000 | 350 |
| 2003 | ++ | − | 200 | 1500 |
| 2102 | +++ | − | 8000 | 800 |
| 2501 | +++ | − | 8000 | 1600 |

Immune sera bound avidly to Aβ plaques and tau tangles in human AD brain sections at concentrations expected to be achieved in CNS under in vivo conditions. Monkeys with the strongest titers at week 26 stained Aβ, and one monkey with low to moderate tau titers stained both Aβ and tau.

These results demonstrate that the serum antibody binding of the cynomolgus monkey antibody response to be balanced and bind to pathological Aβ plaques and tau tangles.

### Example 9: Serum Blocking of Soluble Aβ Aggregates from Binding to Neurons and T-Cell Activity

Primary hippocampal neurons isolated from E18 rat were cultured as described above. Fresh unlabeled, biotinylated or (9:1) soluble Aβ was prepared one day prior to assay and incubated overnight at 4° C. Neurons were rinsed with NB-NPR at 150 μL/well before adding Aβ/serum treatment. An IgG cut of cynomolgus monkey bleeds from vaccinated animals was added to E18 neurons at 60 μL/well, and then incubated for 30 minutes at 37° C. under normal incubator conditions (5% CO₂; 9% O₂). Cells were rinsed twice using 150 μL/well of NB-NPR, and then fixed in 4% paraformaldehyde in 1×DPBS for 20 minutes. Cells were permeabilized using 0.1 TX-100 for 5 minutes, and blocked using 10% normal goat serum (NGS; Thermofisher) for 1 hour at room temperature (RT). Cells were incubated with MAP2 & NeuN primary antibodies in 100 μL/well, in 1×DPBS containing 1% BSA+1% NGS overnight at 4° C.

The next day, cells were rinsed twice in 150 μL/well 1×DPBS for 5 minutes each wash. Secondary antibodies were added for 1 hour at RT in 100 μL/well 1×DPBS+1% BSA+1% NGS. Aβ soluble aggregate spots were detected using streptavidin-488 or polyclonal Aβ antibody (Thermofisher; Millipore); High-content imaging (HCl) analysis was performed to quantify soluble aggregate Aβ neuritic binding using Operetta HCl CLS instrument (Perkin Elmer; modified Neurite Outgrowth algorithm: 40'H₂O objective; 40 fields per well; (n=3) per condition; data shown as mean (+/−) SD); MAP2 & NeuN (Abcam) neuronal markers were used to each trace neurite tree and count cell body number per optical field; Neuritic data were reported as Aβ soluble aggregate spots/neuron (or as Integrated Intensity)). Approximately 80-150 neurons were observed per well for each condition tested (Zago, et al., 2012).

T-cell reactivity was also investigated. Immune sera inhibited the binding of soluble Aβ aggregates to hippocampal neurons. Immunogens did not elicit cytotoxic T-cell responses to Aβ or tau.

### Example 10: Animals Vaccinated with Dual Peptide Antigens Produce Titers to Aβ and Tau

The study described in this example was designed to assess dual Aβ and tau antigen peptides in mice. The dual Aβ-tau constructs in this example effectively demonstrated: high titers for both antigens; blocking of tau binding to heparin; and staining/binding to Aβ and tau peptides in brain tissue from human Alzheimer's patients. Furthermore, titers against tau for an engineered tau immunogen (Dual #11) were comparable, and in some cases, better than other tau immunogens despite including a non-native tau sequence. This demonstrates that engineered immunogens are useful in vaccine constructs.

TABLE 8

| Dual | | Abeta | Endo | | tau | | |
|---|---|---|---|---|---|---|---|
| Con- | | SEQ | pepti- | | SEQ | | |
| struct | Abeta | ID | dase | Tau | ID | C-Terminal | |
| ID # | Sequence | NO. | linker | Sequence | NO. | linker | Cys |
| 2 | DAEFRHD | 06 | RR | VKSKIGST | 801 | GG | C |
| 3 | DAEFRHD | 06 | RR | SKIGSTEN | 817 | GG | C |
| 8 | DAEFRHD | 06 | RR | TENLKHQP | 695 | GG | C |
| 9 | DAEFRHD | 06 | RR | ENLKHQPG | 689 | GG | C |
| 11 | DAEFRHD | 06 | RR | SKIGSKDNIKH | 986 | GG | C |

TABLE 9

| Dual | | |
|---|---|---|
| Construct | | SEQ ID |
| ID # | Sequence | NO: |
| 2 | DAEFRHDRRVKSKIGSTGGC | 997 |
| 3 | DAEFRHDRRSKIGSTENGGC | 998 |
| 8 | DAEFRHDRRTENLKHQPGGC | 999 |
| 9 | DAEFRHDRRENLKHQPGGGC | 1000 |
| 11 | DAEFRHDRRSKIGSKDNIKHGGC | 1001 |

Conjugation—Peptides were made by Biopeptide (San Diego, CA) CRM-bromoacetate was received from Fina Biosolutions (Rockville, MD) peptides were coupled to CRM following the below protocol.

1M Tris HCL pH 8.0, MilliQ DI Water and 50 mM Borate, 100 mM NaCl, 5 mM EDTA pH 8.5 were sterile filtered and degassed. 1 mg of each peptide was dissolved in 0.2 mls of degassed water, then 0.1 mls degassed Tris_HCL was added. This was followed by 0.2 mls of the stock CRM-Bromoacetate (1 mg total), finally 0.5 mls of the Borate buffer was added. This incubated 24 hours at 4 degrees C. on a nutator to provide mixing. Samples were desalted into PBS and 5 μl was run on a 10% Tris gel to confirm conjugation.

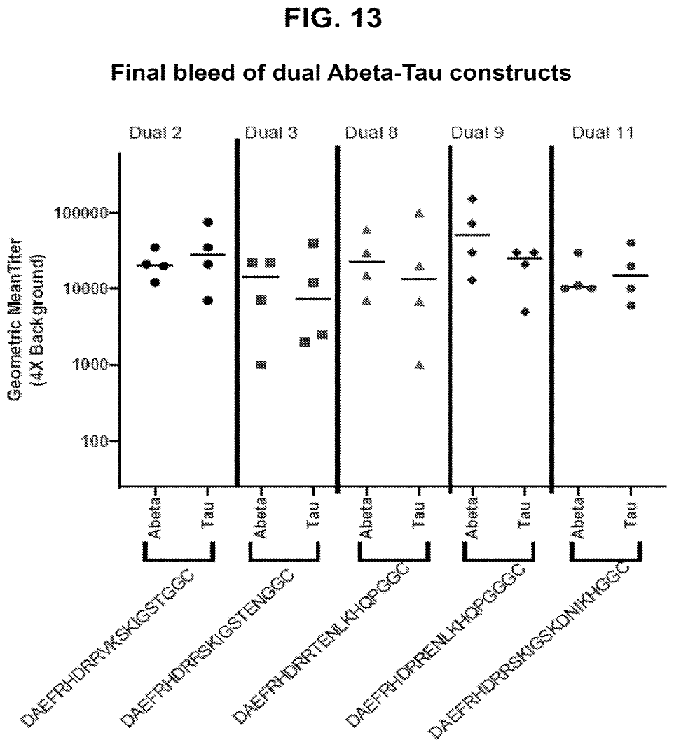

Injection of animals: Four (4) female Swiss webster mice were used in each group. Immunogen preparation was 25 μg Immunogen, 25 μg QS21 and 150 μl of 0.02% Tween 80/PBS per injection. Each mouse received 200 μl subcutaneously. Mice were injected at day 0, at 4 weeks from day 0, and at 8 weeks from day 0, with bleeds taken for titer at 5 weeks from day 0 and animals sacrificed and terminal bleed collected at 9 weeks from day 0 (see FIG. 13).

Titer Assay: Mouse serum was titered by enzyme-linked immunosorbent assay (ELISA). Plates were coated overnight at 2 μg/mL with either Abeta 1-28 (Anaspec, San Jose, CA), or recombinant tau (Proteos, Kalamazoo, MI in phosphate-buffered saline (PBS), and then blocked 1 hour with 1% bovine serum albumin (BSA) in PBS. Normal mouse serum was used as a negative control while known positive anti-serum from previous mouse studies was used as a positive control at the same dilutions as test serum. Bleeds were diluted in PBS/0.1% BSA/0.1% Tween 20 (PBS/BSA/T) starting at 1/100 and serially diluted 1:2 down the plate. Plates were washed with TBS/Tween 20 and goat anti-mouse immunoglobulin G (IgG) (heavy+light chains) horseradish peroxidase (HRP) (ThermoFisher) 1/5000 was added and incubated 1 hour at room temperature. Plates were washed in TBS/Tween 20, and antibody binding was detected with o-phenylenediamine dihydrochloride (OPD) substrate (Thermo Fisher Scientific, Waltham, MA) following manufacturer's instructions. Plates were read at 490 nM on a Molecular Devices Spectromax. Titer was defined as the dilution giving 4× background (defined in graphs and tables); extrapolation was used if it fell in between dilutions.

### MTBR Binding

Certain antibodies that bind to MTBR can bind to more than one MTBR region due to the homology of the various MTBR regions. Antiserum is titered on four MTBR regions using peptides of MTBR 1-4 purchased from Anaspec (San Jose, CA)

MTBR peptide 1
(SEQ ID NO: 1058)
QTAPVPMPDLKNVKSKIGSTENLKHQPGGGK
MTBR peptide 2
(SEQ ID NO: 1059)
VQIINKKLDLSNVQSKCGSKDNIKHVPGGGS
MTBR peptide 3
(SEQ ID NO:1060)
VQIVYKPVDLSKVTSKCGSLGNIHHKPGGGQ
MTBR peptide 4
(SEQ ID NO: 1061)
VEVKSEKLDFKDRVQSKIGSLDNITHVPGGGN

Mouse serum is titered by enzyme-linked immunosorbent assay (ELISA). Plates are coated overnight at 2 μg/mL with each the various MTBR peptides in phosphate-buffered saline (PBS) and then blocked 1 hour with 1% bovine serum albumin (BSA) in PBS. Normal mouse serum is used as a negative control. Bleeds are diluted in PBS/0.1% BSA/0.1% Tween 20 (PBS/BSA/T) starting at 1/100 and serially diluted 1:2 down the plate. Plates are washed with TBS/Tween 20 and goat anti-mouse immunoglobulin G (IgG) (heavy+light chains) horseradish peroxidase (HRP) (ThermoFisher) 1/5000 is added and incubated 1 hour at room temperature. Plates are washed in TBS/Tween 20, and antibody binding is detected with o-phenylenediamine dihydrochloride (OPD) substrate (Thermo Fisher Scientific, Waltham, MA) following manufacturer's instructions. Plates are read at 490 nM on a Molecular Devices Spectromax. Titer is defined as the dilution giving 4× background (defined in graphs and tables); extrapolation is used if it falls in between dilutions.

### Blocking of Tau Binding to Heparin

As a potential surrogate marker for the ability of the serum to block uptake of tau into cells an ELISA measuring the blocking of tau binding to Heparin plates was developed. Recombinant tau was biotinylated in-house. Heparin coated plates (Bioworld, Dublin, OH) were blocked with 2% BSA/PBS for 1 hour. In a separate deep well polypropylene 96 well plate (ThermoFisher) Serum was diluted from 1/25-1/3200 in 2% BSA/PBS, 60 μl total, to this 60 μl 200 ng/ml biotinylated tau in 2% BSA/PBS was added for a final concentration of serum 1/50-1/6400 and tau at 100 ng/ml. The mixture of serum and tau incubated for 2 hours then 100 μl/well was transferred to the blocked heparin plates and incubated 1 hour. Plates were washed in 0.1% Tween 20/TBS and goat anti-mouse immunoglobulin G (IgG) (heavy+light chains) horseradish peroxidase (HRP) (ThermoFisher) 1/5000 was added and incubated 1 hour at room temperature. Plates were washed in TBS/Tween 20 and 100 μl ThermoFisher TMB added and incubated 8 minutes stopped H₂SO₄ and read at 450.

### IHC Binding of Serum

Autopsy blocks of fresh frozen human brain tissue were embedded in optimal cutting temperature compound (OCT compound) and cut using a cryostat to generate 10 μm sections. The sections were placed into a solution of glucose oxidase and beta D-glucose, in the presence of sodium azide, to block endogenous peroxidase. Once tissue sections were prepared, the staining with the specified mouse immune sera was carried out at 1:500 in 5% goat serum with 0.25% triton for 1 hour at RT. To image the binding to plagues and tangles, Biotin-SP-Conjugated Goat anti mouse IgG from Jackson (Lot #115-065-166) at 1:200 dilution were incubated with the sections. DAKO DAB Detection Kit as per the manufacturer's instructions. The staining was processed using an automated Leica Bond Stainer. The results indicate that sera from Guinea pigs immunized with a vaccine as disclosed herein comprises antibodies specific to Abeta and tau in human brain tissue of Alzheimer's patients

### Serum Blocking of Abeta Binding to Neurons

E18 primary rat hippocampal neurons are cultured as described previously (Zago, et al. “Neutralization of Soluble, Synaptotoxic Amyloid β Species by Antibodies Is Epitope Specific,” J Neurosci. 2012 Feb. 22; 32(8): 2696-2702). Soluble Aβ aggregate is pre-incubated with or without vaccine serum on culture DIV14-21 to block soluble Aβ aggregate from neuritic binding. Fresh unlabeled, biotinylated or (9:1) soluble Aβ is prepared one day prior and incubated overnight at 4° C. Each diluted serum sample and soluble Aβ solution is prepared at 2× the final concentration in one-half of final treatment volume using NeuroBasal-no phenol red (NB-NPR) medium. This is combined with one-half final volume of 2× soluble Aβ and with one-half final volume of 2× diluted vaccine serum to make up a 1× final concentration in total final treatment volume, which is mixed well and then pre-incubated for 30 minutes at 37° C. E18 neurons are rinsed with NB-NPR at 150 μL/well before adding binding treatment. Serum from vaccinated animals/Aβ treatment to is added to E18 neurons at 60 μL/well, and then incubated for 30 minutes at 37° C. under normal incubator conditions (5% CO₂; 9% O₂). Cells are rinsed twice using 150 μL/well of NB-NPR, and then fixed in 4% paraformaldehyde in 1×DPBS for 20 minutes. Cells are permeabilized using 0.1 TX-100 for 5 minutes, and blocked using 10% normal goat serum (NGS) for 1 hour at room temperature (RT). Cells are incubated with MAP2 & NeuN primary antibodies in 100 μL/well, 1×DPBS containing 1% BSA+1% NGS overnight at 4° C. The next day, cells are rinsed twice in 150 μL/well 1×DPBS for 5 minutes each wash. Secondary antibodies are added for 1 hour at RT in 100 μL/well 1×DPBS+1% BSA+1% NGS. High-content imaging (HCl) analysis is performed to quantify soluble aggregate Aβ neuritic binding using Operetta HCl CLS instrument (Perkin Elmer; modified Neurite Outgrowth algorithm: 40× H₂O objective; 40 fields per well; (n=3) per condition; data shown as mean (+/−) SD); MAP2 & NeuN (Abcam) neuronal markers is used to each trace neurite tree and count cell body number per optical field; Neuritic Aβ soluble aggregate spots detected using streptavidin-488 or polyclonal Aβ antibody (Thermo; Millipore); and data reported as Aβ soluble aggregate spots/neuron (or as Integrated Intensity)). Approximately 80-150 neurons are observed per well for each condition tested.

### Results

Data for Abeta and tau titers and staining is summarized in Table 10 and Table 11.

TABLE 10

| | | | | 50% | | |
|---|---|---|---|---|---|---|
| | | Titer | Titer | blocking | IHC | IHC |
| | Animal | Abeta | Tau | tau binding | Abeta | Tau |
| Dual | 2.1 | 35,000 | 35,000 | 1:300 | ++++ | + |
| Construct | 2.2 | 21,000 | 75,000 | 1:225 | ++++ | + |
| 2 | 2.3 | 12,000 | 21,000 | 1:550 | +++ | +++++ |
| | 2.4 | 20,000 | 7,000 | 1:200 | ++ | +++++ |
| Dual | 3.1 | 22,000 | 12,000 | 1:275 | +++ | ++++ |
| Construct | 3.2 | 20,000 | 2,000 | 1:200 | ~ | ++++ |
| 3 | 3.3 | 7,000 | 40,000 | 1:125 | ++++ | ++ |
| | 3.4 | 1,000 | 25,000 | 1:200 | + | ++++ |
| Dual | 8.1 | 15,000 | 100,000 | 1:275 | ++ (or +½) | ++++½ |
| Construct | 8.2 | 7,000 | 6,800 | 1:675 | ++ (or +½) | +++ |
| 8 | 8.3 | 60,000 | 10,000 | 1:200 | +++ | − |
| | 8.4 | 30,000 | 20,000 | 1:200 | ++++ | ++++ |
| Dual | 9.1 | 150,000 | 50,000 | 1:525 | + | ++++½ |
| Construct | 9.2 | 30,000 | 21,000 | 1:325 | ++ | ++++ |
| 9 | 9.3 | 13,000 | 30,000 | 1:325 | +++ | ++++ |
| | 9.4 | 72,000 | 30,000 | 1:625 | +++ | +++ |
| Dual | 11.1 | 30,000 | 6,000 | 1:375 | ++++ | ++½ |
| Construct | 11.2 | 10,000 | 40,000 | 1:875 | ~ | ++++ |
| 11 | 11.3 | 11,000 | 20,000 | 1:325 | ++++½ | ++ |
| | 11.4 | 10,000 | 10,000 | 1:275 | ++++ | + |

TABLE 11

| Dual | | | | |
|---|---|---|---|---|
| Abeta/Tau | | Tau | | |
| Mouse | | Cell | | |
| Sera | A-beta | bodies | Neurites | Notes |
| 2.1 | ++++ | + | + | |
| 2.2 | ++++ | ++ | + | Presence of nuclear staining |
| 2.3 | +++ | +++++ | +++ | |
| 2.4 | ++ | +++++ | ++++ | |
| 3.1 | +++ | ++++ | +++++ | |
| 3.2 | − | ++++ | +++++ | |
| 3.3 | ++++ | ++ | +++ | Strong nuclear staining |
| 3.4 | + | ++++ | ++ | |
| 8.1 | ++ | ++++ | ++++ | |
| 8.2 | ++ | +++ | +++ | |
| 8.3 | +++ | − | − | |
| 8.4 | ++++ | ++++ | +++++ | |
| 9.1 | + | ++++ | ++++ | |
| 9.2 | ++ | ++++ | +++ | |
| 9.3 | +++ | ++++ | ++++ | |
| 9.4 | +++ | +++ | ++++ | |
| 11.1 | ++++ | ++ | +++ | |
| 11.2 | + | ++++ | + | |
| 11.3 | +++++ | ++ | − | |
| 11.4 | ++++ | + | − | Some nuclear staining |

### Titers

As seen in FIG. 13 and Tables 10-13, all groups most animals produced high and balanced Abeta and tau titers, though some had a less variable titer response.

TABLE 12
Titers for the dual Abeta/tau constructs.

| | Abeta | | | | tau | | | |
|---|---|---|---|---|---|---|---|---|
| | Mouse | Mouse | Mouse | Mouse | Mouse | Mouse | Mouse | Mouse |
| | 1 | 2 | 3 | 4 | 1 | 2 | 3 | 4 |
| Dual | | | | | | | | |
| Construct 2 | | | | | | | | |
| Bleed 1 | 30000 | 25000 | 25000 | 12000 | 150 | 5000 | 3000 | 4000 |
| Bleed 2 | 35000 | 21000 | 12000 | 20000 | 35000 | 75000 | 21000 | 7000 |
| Dual | | | | | | | | |
| Construct 3 | | | | | | | | |
| Bleed 1 | 6400 | 800 | 15000 | 100 | 4000 | 6400 | 5000 | 2000 |
| Bleed 2 | 22000 | 22000 | 7000 | 1000 | 12000 | 2000 | 40000 | 2500 |
| Dual | | | | | | | | |
| Construct 8 | | | | | | | | |
| Bleed 1 | 1500 | 2000 | 20000 | 18000 | 10000 | 25000 | 200 | 8000 |
| Bleed 2 | 15000 | 7000 | 60000 | 30000 | 100000 | 6800 | 1000 | 20000 |
| Dual | | | | | | | | |
| Construct 9 | | | | | | | | |
| Bleed 1 | 2000 | 12000 | 13000 | 6400 | 800 | 6000 | 7000 | 2000 |
| Bleed 2 | 150000 | 30000 | 13000 | 72000 | 5000 | 21000 | 30000 | 30000 |
| Dual | | | | | | | | |
| Construct 11 | | | | | | | | |
| Bleed 1 | 6000 | 2000 | 5000 | 7000 | 6400 | 7000 | 2500 | 4000 |
| | 30000 | 10000 | 11000 | 10000 | 6000 | 40000 | 20000 | 10000 |

TABLE 13
Remaining tau bound after serum incubation

| | DAEFRHDRRVKSKIGSTGGC | | | |
|---|---|---|---|---|
| | (SEQ ID NO: 997) | | | |
| dilution | Mouse 1 | Mouse 2 | Mouse 3 | Mouse 4 |
| 50 | 19.57 | 29.12 | 16.66 | 12.70 |
| 100 | 28.93 | 38.25 | 26.51 | 25.21 |
| 200 | 41.16 | 53.48 | 29.64 | 46.53 |
| 400 | 59.88 | 89.43 | 43.15 | 81.31 |
| 800 | 95.14 | 139.21 | 65.00 | 134.97 |
| | DAEFRHDRRENLKHQPGGGC | | | |
| | (SEQ ID NO: 1000) | | | |
| dilution | Mouse 1 | Mouse 2 | Mouse 3 | Mouse 4 |
| 50 | 16.44 | 21.90 | 24.26 | 15.35 |
| 100 | 22.07 | 47.55 | 39.93 | 19.39 |
| 200 | 37.10 | 44.06 | 48.36 | 43.65 |
| 400 | 58.32 | 86.61 | 86.41 | 51.98 |
| 800 | 93.54 | 98.76 | 115.50 | 86.00 |
| | DAEFRHDRRSKIGSTENGGC | | | |
| | (SEQ ID NO: 998) | | | |
| dilution | Mouse 1 | Mouse 2 | Mouse 3 | Mouse 4 |
| 50 | 16.84 | 27.29 | 28.01 | 28.56 |
| 100 | 25.18 | 39.02 | 53.18 | 42.60 |
| 200 | 55.66 | 73.61 | 93.32 | 75.38 |
| 400 | 92.87 | 120.00 | 131.15 | 125.27 |
| 800 | 110.16 | 132.77 | 130.90 | 135.93 |
| | DAEFRHDRRSKIGSKDNIKHGGC | | | |
| | (SEQ ID NO: 1001) | | | |
| dilution | Mouse 1 | Mouse 2 | Mouse 3 | Mouse 4 |
| 50 | 15.25 | 13.15 | 14.73 | 17.81 |
| 100 | 19.45 | 11.20 | 26.64 | 37.79 |
| 200 | 37.65 | 14.59 | 56.17 | 54.57 |
| 400 | 74.17 | 39.69 | 78.69 | 91.10 |
| 800 | 90.35 | 62.16 | 96.30 | 109.00 |
| | DAEFRHDRRTENLKHQPGGC | | | |
| | (SEQ ID NO: 999) | | | |
| dilution | Mouse 1 | Mouse 2 | Mouse 3 | Mouse 4 |
| 50 | 20.01 | 12.36 | 35.04 | 42.63 |
| 100 | 37.62 | 28.44 | 44.45 | 47.62 |
| 200 | 54.86 | 47.97 | 66.72 | 61.84 |
| 400 | 114.93 | 48.51 | 91.94 | 87.67 |
| 800 | 110.60 | 81.25 | 100.01 | 97.15 |
| | Negative Control | | | |
| dilution | Mouse 1 | Mouse 2 | Mouse 3 | Mouse 4 |
| 50 | 83.35 | 71.98 | 84.30 | 83.96 |
| 100 | 114.78 | 94.03 | 103.43 | 98.98 |
| 200 | 104.03 | 100.60 | 97.80 | 96.83 |
| 400 | 86.73 | 111.16 | 95.31 | 91.72 |
| 800 | 96.20 | 110.05 | 99.38 | 94.45 |

### Blocking of Tau Binding to Heparin

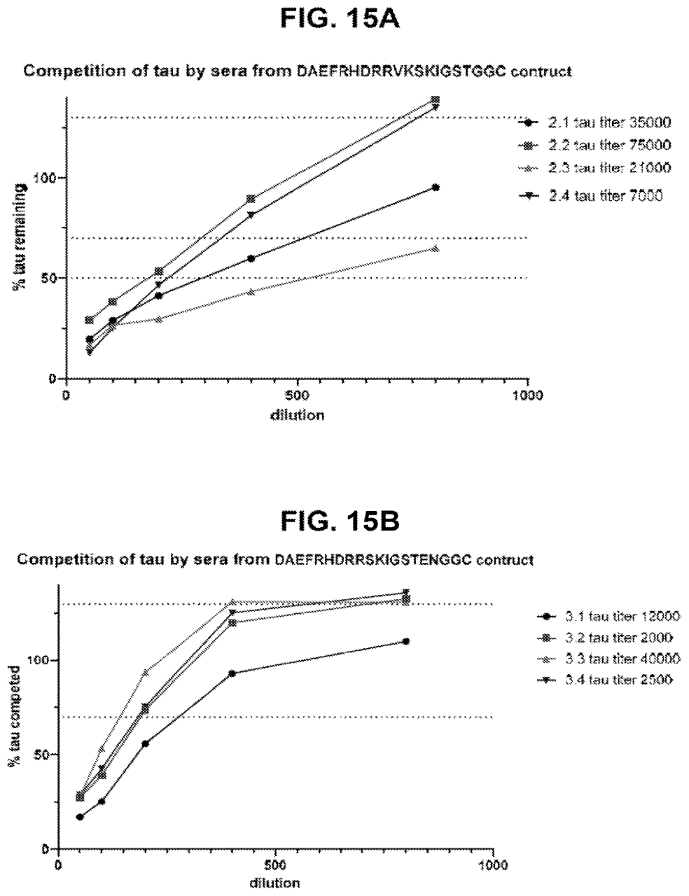

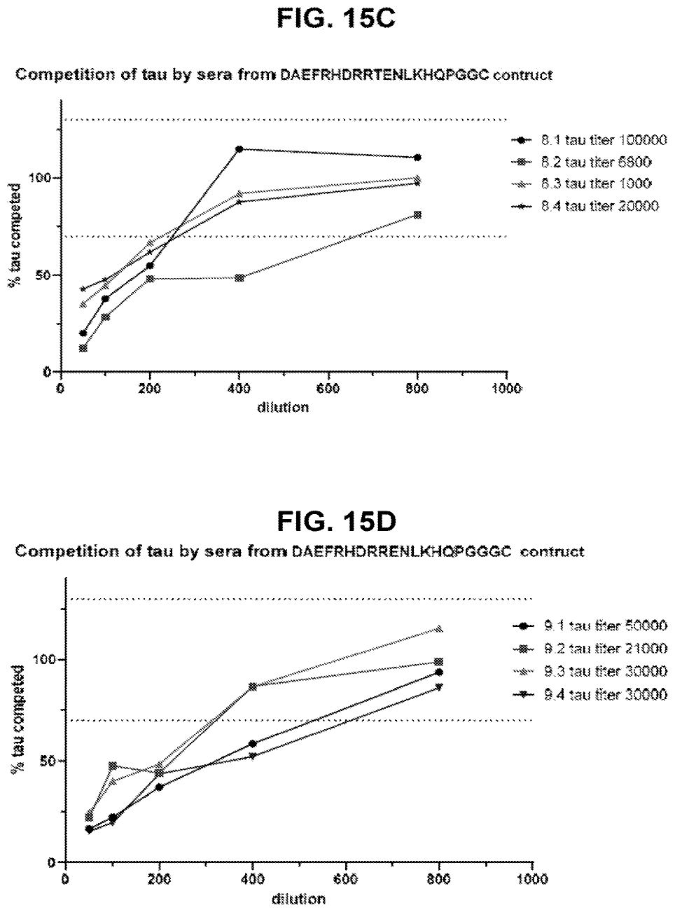

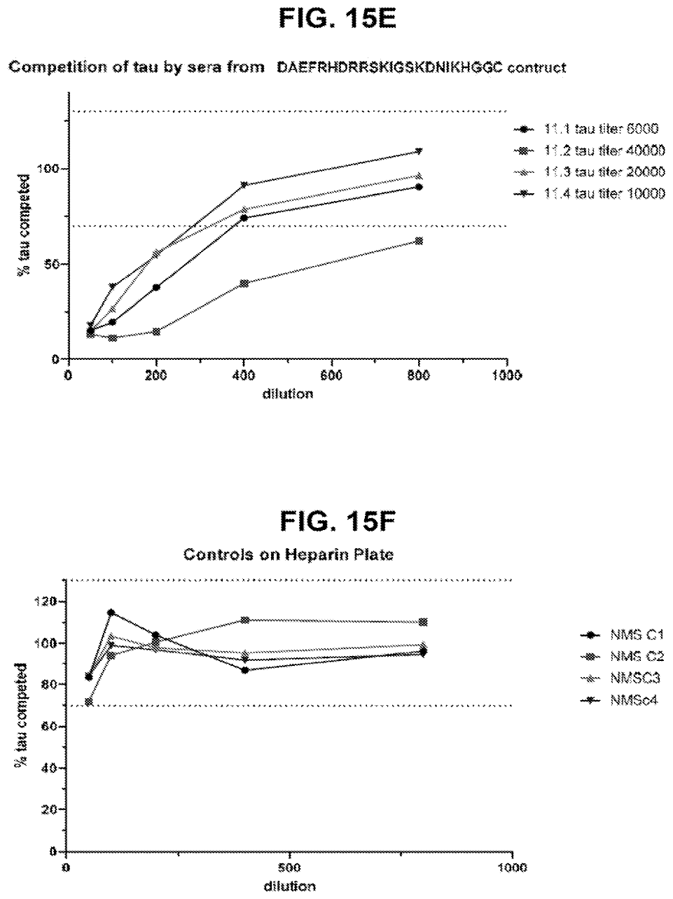

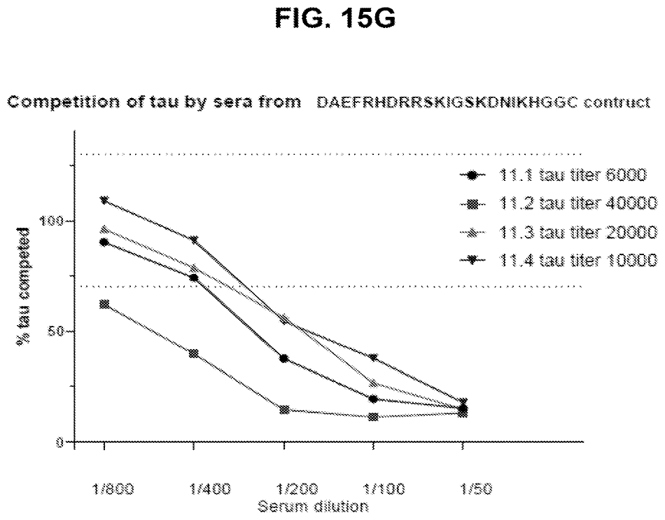

All constructs showed the ability to block the binding of tau to heparin, in particular, construct DAEFRHDRRVKSKIGSTGGC (SEQ ID NO:997) and construct DAEFRHDRRENLKHQPGGGC (SEQ ID NO:1000) showing the most robust blocking. See FIG. 15, Table 10 and Table 11.

### IHC on Alzheimer Brain

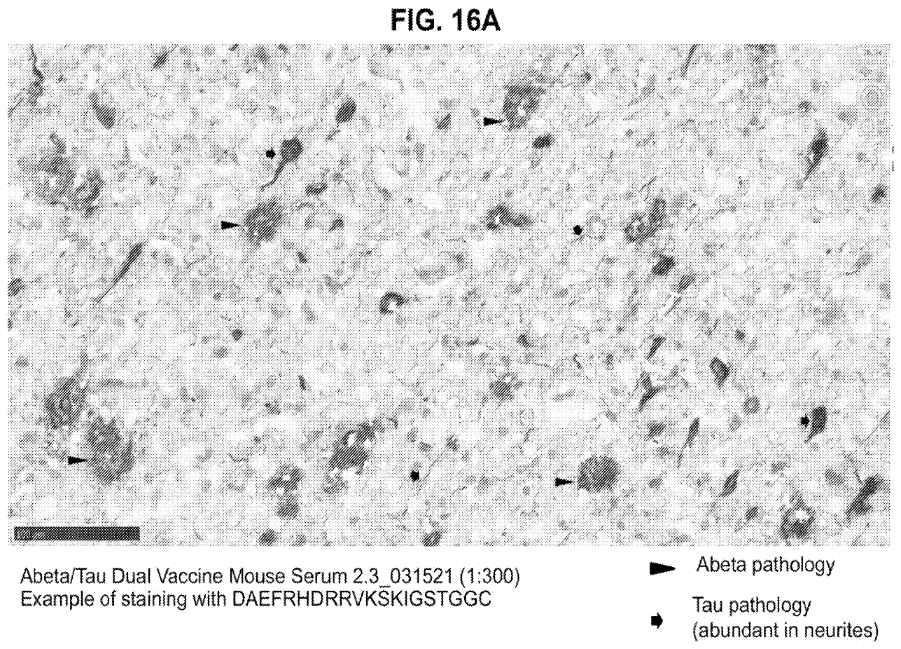

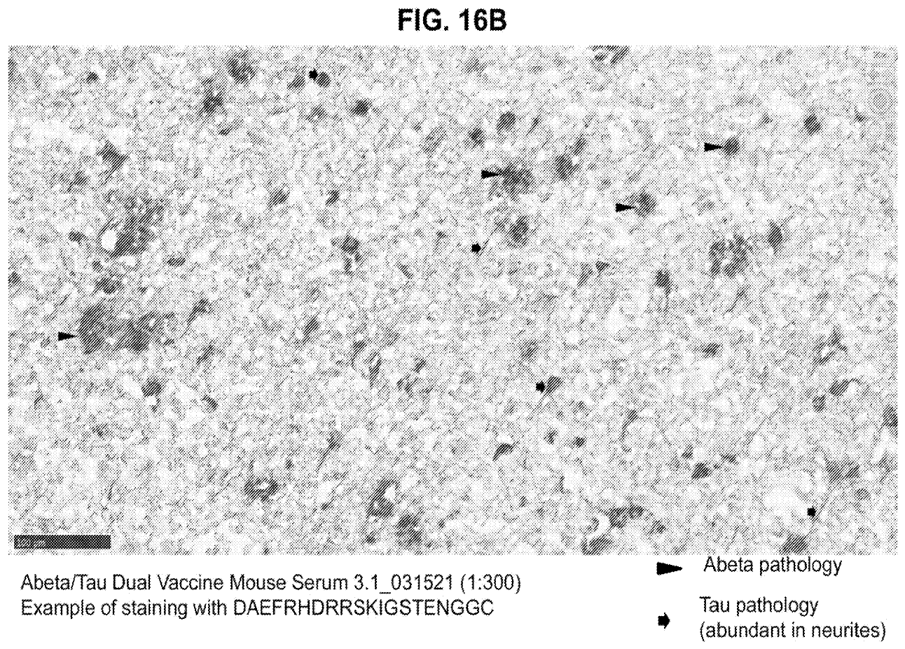

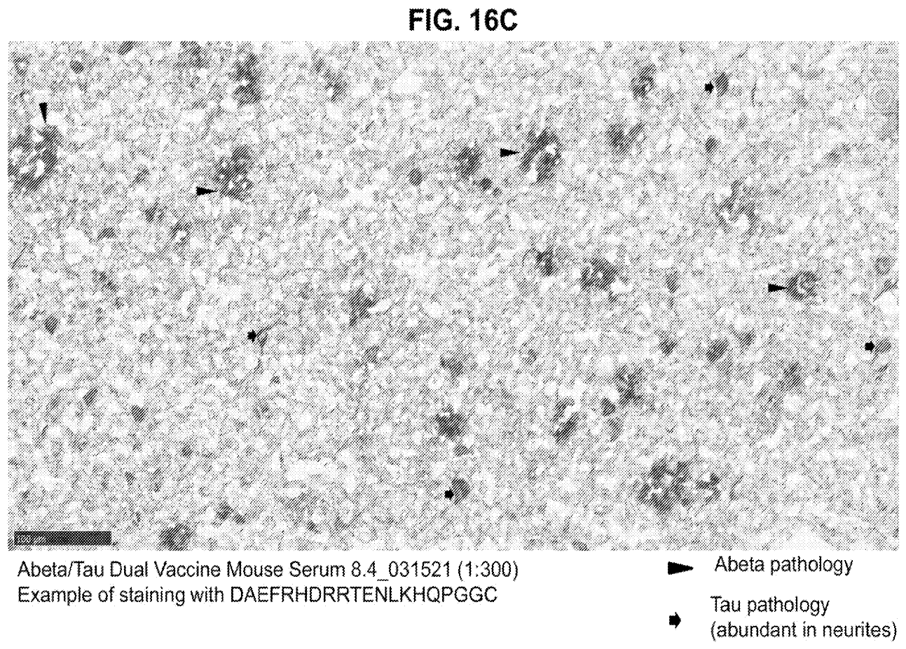

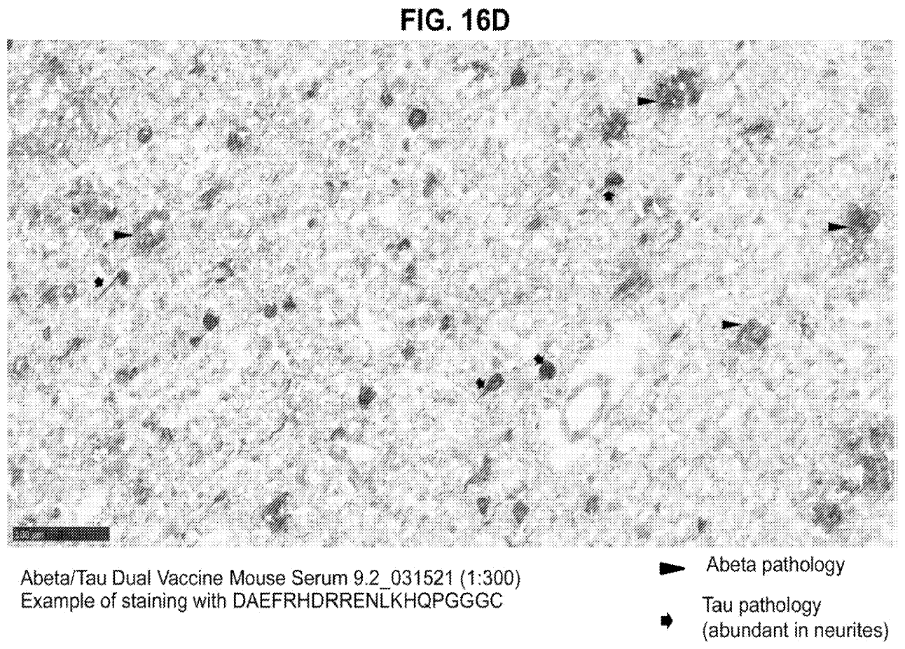

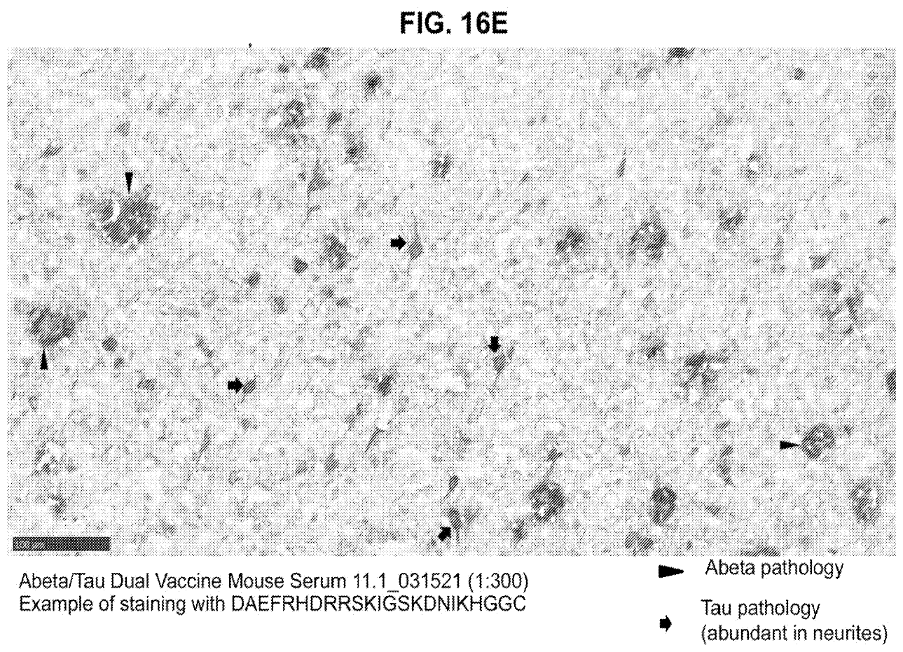

Most animals had staining of pathological Abeta and tau, see FIG. 16.

### Example 11: Immunogens

Immunogens were selected for evaluation in vaccine peptide constructs. Constructs comprise Aβ immunogens and tau immunogens. Some immunogens comprise a tau peptide comprising 3-10 amino acids from tau. Other immunogens comprise an engineered tau immunogen.

### Engineered Tau Immunogens

Certain immunogenic peptides were designed and selected to (i) raise antibodies that bind within the within microtubule binding repeats (MTBRs) of human Tau protein, (ii) be less likely to generate an unwanted T cell-mediated autoimmune response, and (iii) be less likely to raise antibodies that would cross-react with other human proteins.

First, sequence analysis and 3D modeling of Tau MTBRs was conducted to identify amino acid residues that may be important for raising antibodies that bind MTBR. The results of these analyses were used to design synthetic Tau immunogenic peptides with conserved residues and shuffled interspersed residues. Resulting engineered synthetic peptides are listed in Table 14.

TABLE 14

| Engineered Tau immunogenic peptides | |
|---|---|
| Engineered tau immunogenic | SEQ ID |
| peptides sequence | NO: |
| SKIGSTENLKH | 978 |
| SKIGSTENIKH | 979 |
| SKIGSKDNLKH | 980 |
| SKIGSKENIKH | 981 |
| SKIGSLENLKH | 982 |
| SKIGSLENIKH | 983 |
| SKIGSTDNLKH | 984 |
| SKIGSTDNIKH | 985 |
| SKIGSKDNIKH | 986 |
| SKIGSLDNLKH | 987 |
| SKIGSLDNIKH | 988 |
| SKIGSTGNLKH | 989 |
| SKIGSTGNIKH | 990 |
| SKIGSKGNLKH | 991 |
| SKIGSKGNIKH | 992 |
| SKIGSLGNLKH | 993 |
| SKIGSLGNIKH | 994 |

Next, to assess the potential of an unwanted T cell-mediated autoimmune response, the engineered peptides were subjected to in silico analysis to predict MHC II binding using the IEDB (Immune Epitope Database) from National Institute of Allergy and Infectious Diseases/La Jolla Immunology Institute. MHC class II binding is considered a good indicator of a sequence containing a T-cell epitope. A panel of alleles were used for MHC II binding prediction. Engineered peptides with a predicted half maximal inhibitory concentration (IC50) above a specified cutoff were considered to have a low probability of MHC II binding and were selected for further analysis.

Finally, the engineered peptides with low predicted MHC II binding were evaluated to predict if the anti-Tau MTBR antibodies that would be raised by the peptides could have unwanted cross-reactivity with other human proteins. Sequences of the engineered peptides were subjected to bioinformatic analysis against a non-redundant human proteome database to determine homology with human proteins. Engineered peptide sequences with low homology to secreted or cell-surface proteins were selected as top candidates to be used as antigens. Top candidate engineered Tau immunogenic peptides are listed in Table 15.

TABLE 15

| Top candidate engineered Tau immunogenic peptides. | |
|---|---|
| Engineered tau immunogenic | SEQ ID |
| peptides sequence | NO: |
| SKIGSTDNIKH | 985 |
| SKIGSKDNIKH | 986 |
| SKIGSLDNIKH | 988 |

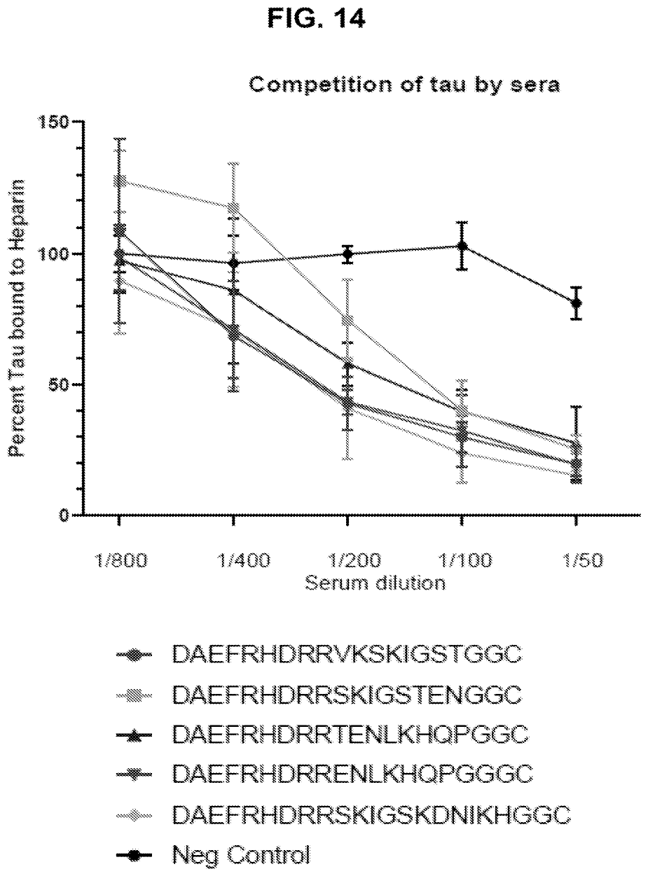

The dual Aβ-tau constructs comprising an engineered tau immunogen (e.g., Dual #11) demonstrated results that were comparable, and in some cases, better than other tau immunogens despite including a non-native tau sequence. FIG. 13 shows high titers for both Abeta and tau antigens, including an engineered tau sequence. FIG. 14 shows blocking of tau binding to heparin. FIG. 15G shows resulting tau antibody binding to MTBR region peptides. FIG. 16 E shows staining/binding to Aβ and tau peptides in brain tissue from human Alzheimer's patients. This demonstrates that engineered immunogens are useful in vaccine constructs

### CONCLUSION

Dual immunogen Aβ-tau vaccine constructs were developed and it was shown that these constructs raised balanced titers to Aβ and tau in mice, guinea-pigs, and cynomolgus monkeys. The antibodies were immunoreactive with both Aβ plaques and neurofibrillary tau tangles in human AD brain sections and blocked the binding of soluble Aβ aggregates (oligomers) to neurons without eliciting T-cell responses for Aβ or tau. These results support the development of a single-agent, dual-immunogen vaccine with the ability to target the pathogenic forms of Aβ and tau. These results support the development of a dual Aβ-tau vaccine with the ability to target pathogenic Aβ and tau for the prevention and/or treatment of AD.

Although various specific embodiments of the present invention have been described herein, it is to be understood that the invention is not limited to those precise embodiments and that various changes or modifications can be affected therein by one skilled in the art without departing from the scope and spirit of the invention.

In each of the embodiments of the peptide described herein, the peptide may comprise, consist, or consist essentially of the recited sequences. Thus, incorporated in this disclosure (see Table 16) are the following sequences that can be part of the compositions comprising an amyloid-beta (Aβ) peptide and a tau peptide as disclosed herein.

TABLE 16
SEQUENCES

```
SEQ ID NO: 01 - Abeta 1-42
DAEFRHDSGYEVHHQKLVFFAEDVGSNKGAIIGLMVGGVVIA
SEQ ID NO: 02 - TAU (UNIPROTKB - P10636
(Homo sapiens)
>P10636-8 (Isoform Tau-F)
                10 20 30 40
MAEPRQEFEV MEDHAGTYGL GDRKDQGGYT MHQDQEGDTD
                50 60 70 80
AGLKESPLQT PTEDGSEEPG SETSDAKSTP TAEDVTAPLV
                90 100 110 120
DEGAPGKQAA AQPHTEIPEG TTAEEAGIGD TPSLEDEAAG
              130 140 150 160
HVTQARMVSK SKDGTGSDDK KAKGADGKTK IATPRGAAPP
              170 180 190 200
GQKGQANATR IPAKTPPAPK TPPSSGEPPK SGDRSGYSSP
              210 220 230 240
GSPGTPGSRS RTPSLPTPPT REPKKVAVVR TPPKSPSSAK
              250 260 270 280
SRLQTAPVPM PDLKNVKSKI GSTENLKHQP GGGKVQIINK
              290 300 310 320
KLDLSNVQSK CGSKDNIKHV PGGGSVQIVY KPVDLSKVTS
              330 340 350 360
KCGSLGNIHH KPGGGQVEVK SEKLDFKDRV QSKIGSLDNI
              370 380 390 400
THVPGGGNKK IETHKLTFRE NAKAKTDHGA EIVYKSPVVS
              410 420 430 440
GDTSPRHLSN VSSTGSIDMV DSPQLATLAD EVSASLAKQG
L
A-beta immunogens:
```

| | |
|---|---|
| DAEFRHDSGY | (SEQ ID NO: 03) |
| DAEFRHDSG | (SEQ ID NO: 04) |
| DAEFRHDS | (SEQ ID NO: 05) |
| DAEFRHD | (SEQ ID NO: 06) |
| DAEFRH | (SEQ ID NO: 07) |
| DAEFR | (SEQ ID NO: 08) |
| DAEF | (SEQ ID NO: 09) |
| DAE | |
| AEFRHDSGY | (SEQ ID NO: 11) |
| AEFRHDSG | (SEQ ID NO: 12) |
| AEFRHDS | (SEQ ID NO: 13) |
| AEFRHD | (SEQ ID NO: 14) |
| AEFRH | (SEQ ID NO: 15) |
| AEFR | (SEQ ID NO: 16) |
| AEF | |
| EFRHDSGY | (SEQ ID NO: 18) |
| EFRHDSG | (SEQ ID NO: 19) |
| EFRHDS | (SEQ ID NO: 20) |
| EFRHD | (SEQ ID NO: 21) |
| EFRH | (SEQ ID NO: 22) |
| EFR | |
| FRHDSGY | (SEQ ID NO: 24) |
| FRHDSG | (SEQ ID NO: 25) |
| FRHDS | (SEQ ID NO: 26) |
| FRHD | (SEQ ID NO: 27) |
| FRH | |
| RHDSGY | (SEQ ID NO: 29) |
| RHDSG | (SEQ ID NO: 30) |
| RHDS | (SEQ ID NO: 31) |
| RHD | |
| HDSGY | (SEQ ID NO: 33) |
| HDSG | (SEQ ID NO: 34) |
| HDS | |
| DSGY | (SEQ ID NO: 36) |
| DSG | |
| SGY | |
| VHHQKLVFFA | (SEQ ID NO: 1002) |
| VHHQKLVFF | (SEQ ID NO: 1003) |
| VHHQKLVF | (SEQ ID NO: 1004) |
| VHHQKLV | (SEQ ID NO: 1005) |
| VHHQKL | (SEQ ID NO: 1006) |
| HHQKLVFFAE | (SEQ ID NO: 1007) |
| HHQKLVFFA | (SEQ ID NO: 1008) |
| HHQKLVFF | (SEQ ID NO: 1009) |
| HHQKLVF | (SEQ ID NO: 1010) |
| HHQKLV | (SEQ ID NO: 1011) |
| HHQKL | (SEQ ID NO: 1012) |
| HQKLVFFAED | (SEQ ID NO: 1013) |
| HQKLVFFAE | (SEQ ID NO: 1014) |
| HQKLVFFA | (SEQ ID NO: 1015) |
| HQKLVFF | (SEQ ID NO: 1016) |
| HQKLVF | (SEQ ID NO: 1017) |
| HQKLV | (SEQ ID NO: 1018) |
| HQKL | (SEQ ID NO: 1019) |
| QKLVFFAEDV | (SEQ ID NO: 1020) |
| QKLVFFAED | (SEQ ID NO: 1021) |
| QKLVFFAE | (SEQ ID NO: 1022) |
| QKLVFFA | (SEQ ID NO: 1023) |
| QKLVFF | (SEQ ID NO: 1024) |
| QKLVF | (SEQ ID NO: 1025) |
| QKLV | (SEQ ID NO: 1026) |
| QKL | |
| KLVFFAEDVG | (SEQ ID NO: 1028) |
| KLVFFAEDV | (SEQ ID NO: 1029) |
| KLVFFAED | (SEQ ID NO: 1030) |
| KLVFFAE | (SEQ ID NO: 1031) |
| KLVFFA | (SEQ ID NO: 1032) |
| KLVFF | (SEQ ID NO: 1033) |
| KLVF | (SEQ ID NO: 1034) |
| KLV | |
| LVFFAEDVG | (SEQ ID NO: 1036) |
| LVFFAEDV | (SEQ ID NO: 1037) |
| LVFFAED | (SEQ ID NO: 1038) |
| LVFFAE | (SEQ ID NO: 1039) |
| LVFFA | (SEQ ID NO: 1040) |
| LVFF | (SEQ ID NO: 1041) |
| LVF | |
| VFFAEDVG | (SEQ ID NO: 1043) |
| VFFAEDV | (SEQ ID NO: 1044) |
| VFFAED | (SEQ ID NO: 1045) |
| VFFAE | (SEQ ID NO: 1046) |
| VFFA | (SEQ ID NO: 1047) |
| VFF | |
| FFAEDVG | (SEQ ID NO: 1049) |
| FFAEDV | (SEQ ID NO: 1050) |
| FFAED | (SEQ ID NO: 1051) |
| FFAE | (SEQ ID NO: 1052) |
| FFA | |
| FAEDVG | (SEQ ID NO: 1054) |
| FAEDV | (SEQ ID NO: 1055) |
| FAED | (SEQ ID NO: 1056) |
| FAE | |

Tau immunogens:

| QIVYKPV | (SEQ ID NO: 39) |
|---|---|
| QIVYKP | (SEQ ID NO: 40) |

| | |
|---|---|
| QIVYKSV | (SEQ ID NO: 41) |
| EIVYKSV | (SEQ ID NO: 42) |
| QIVYKS | (SEQ ID NO: 83) |
| EIVYKSP | (SEQ ID NO: 43) |
| EIVYKS | (SEQ ID NO: 84) |
| EIVYKPV | (SEQ ID NO: 44) |
| EIVYKP | (SEQ ID NO: 85) |
| IVYKSPV | (SEQ ID NO: 45) |
| IVYK | (SEQ ID NO: 46) |
| CNIKHVPG | (SEQ ID NO: 86) |
| CNIKHVP | (SEQ ID NO: 47) |
| NIKHVP | (SEQ ID NO: 48) |
| HVPGGG | (SEQ ID NO: 49) |
| HVPGG | (SEQ ID NO: 50) |
| HKPGGG | (SEQ ID NO: 51) |
| HKPGG | (SEQ ID NO: 52) |
| KHVPGGG | (SEQ ID NO: 53) |
| KHVPGG | (SEQ ID NO: 54) |
| HQPGGG | (SEQ ID NO: 55) |
| HQPGG | (SEQ ID NO: 56) |
| VQIINK | (SEQ ID NO: 146) |
| VQIINKK | (SEQ ID NO: 147) |
| VQIINKKL | (SEQ ID NO: 148) |
| QIINK | (SEQ ID NO: 149) |
| QIINKK | (SEQ ID NO: 150) |
| QIINKKL | (SEQ ID NO: 151) |
| EAAGHVTQC | (SEQ ID NO: 152) |
| EAAGHVTQAR | (SEQ ID NO: 153) |
| AAGHVTQAC | (SEQ ID NO: 154) |
| AGHVTQARC | (SEQ ID NO: 155) |
| AGHVTQAR | (SEQ ID NO: 156) |
| GYTMHQD | (SEQ ID NO: 157) |
| QGGYTMHC | (SEQ ID NO: 158) |
| QGGYTMHQD | (SEQ ID NO: 159) |
| GGYTMHQC | (SEQ ID NO: 160) |
| VPGGGSVQIV | (SEQ ID NO: 161) |
| PGGGSVQIV | (SEQ ID NO: 162) |
| GGGSVQIV | (SEQ ID NO: 163) |
| GGSVQIV | (SEQ ID NO: 164) |
| GSVQIV | (SEQ ID NO: 165) |
| SVQIV | (SEQ ID NO: 166) |
| VQIV | (SEQ ID NO: 167) |
| QIV | |
| PGGGSVQIVY | (SEQ ID NO: 169) |
| GGGSVQIVY | (SEQ ID NO: 170) |
| GGSVQIVY | (SEQ ID NO: 171) |
| GSVQIVY | (SEQ ID NO: 172) |
| SVQIVY | (SEQ ID NO: 173) |
| VQIVY | (SEQ ID NO: 174) |
| QIVY | (SEQ ID NO: 175) |
| IVY | |
| GGGSVQIVYK | (SEQ ID NO: 177) |
| GGSVQIVYK | (SEQ ID NO: 178) |
| GSVQIVYK | (SEQ ID NO: 179) |
| SVQIVYK | (SEQ ID NO: 180) |
| VQIVYK | (SEQ ID NO: 181) |
| QIVYK | (SEQ ID NO: 182) |
| VYK | |
| GGSVQIVYKP | (SEQ ID NO: 184) |
| GSVQIVYKP | (SEQ ID NO: 185) |
| SVQIVYKP | (SEQ ID NO: 186) |
| VQIVYKP | (SEQ ID NO: 187) |
| IVYKP | (SEQ ID NO: 188) |
| VYKP | (SEQ ID NO: 189) |
| YKP | |
| GSVQIVYKPV | (SEQ ID NO: 191) |
| SVQIVYKPV | (SEQ ID NO: 192) |
| VQIVYKPV | (SEQ ID NO: 193) |
| IVYKPV | (SEQ ID NO: 194) |
| VYKPV | (SEQ ID NO: 195) |
| YKPV | (SEQ ID NO: 196) |
| KPV | |
| SVQIVYKPVD | (SEQ ID NO: 198) |
| VQIVYKPVD | (SEQ ID NO: 199) |
| QIVYKPVD | (SEQ ID NO: 200) |
| IVYKPVD | (SEQ ID NO: 201) |
| VYKPVD | (SEQ ID NO: 203) |
| YKPVD | (SEQ ID NO: 204) |
| KPVD | (SEQ ID NO: 205) |
| PVD | |
| VQIVYKPVDL | (SEQ ID NO: 207) |
| QIVYKPVDL | (SEQ ID NO: 208) |
| IVYKPVDL | (SEQ ID NO: 209) |
| VYKPVDL | (SEQ ID NO: 210) |
| YKPVDL | (SEQ ID NO: 211) |
| KPVDL | (SEQ ID NO: 212) |
| PVDL | (SEQ ID NO: 213) |
| VDL | |
| QIVYKPVDLS | (SEQ ID NO: 215) |
| IVYKPVDLS | (SEQ ID NO: 216) |
| VYKPVDLS | (SEQ ID NO: 217) |
| YKPVDLS | (SEQ ID NO: 218) |
| KPVDLS | (SEQ ID NO: 219) |
| PVDLS | (SEQ ID NO: 220) |
| VDLS | (SEQ ID NO: 221) |
| IVYKPVDLSK | (SEQ ID NO: 223) |
| VYKPVDLSK | (SEQ ID NO: 224) |
| YKPVDLSK | (SEQ ID NO: 225) |
| KPVDLSK | (SEQ ID NO: 226) |
| PVDLSK | (SEQ ID NO: 227) |
| VDLSK | (SEQ ID NO: 228) |
| DLSK | (SEQ ID NO: 229) |
| LSK | |
| VYKPVDLSKV | (SEQ ID NO: 231) |
| YKPVDLSKV | (SEQ ID NO: 232) |
| KPVDLSKV | (SEQ ID NO: 233) |
| PVDLSKV | (SEQ ID NO: 234) |
| VDLSKV | (SEQ ID NO: 235) |
| DLSKV | (SEQ ID NO: 236) |
| LSKV | (SEQ ID NO: 237) |
| SKV | |
| YKPVDLSKVT | (SEQ ID NO: 239) |
| KPVDLSKVT | (SEQ ID NO: 240) |
| PVDLSKVT | (SEQ ID NO: 241) |
| VDLSKVT | (SEQ ID NO: 242) |
| AKTDHGAEIV | (SEQ ID NO: 243) |
| KTDHGAEIV | (SEQ ID NO: 244) |
| TDHGAEIV | (SEQ ID NO: 245) |
| DHGAEIV | (SEQ ID NO: 246) |
| HGAEIV | (SEQ ID NO: 247) |
| GAEIV | (SEQ ID NO: 248) |
| AEIV | (SEQ ID NO: 249) |
| EIV | |
| KTDHGAEIVY | (SEQ ID NO: 251) |
| TDHGAEIVY | (SEQ ID NO: 252) |
| DHGAEIVY | (SEQ ID NO: 253) |
| HGAEIVY | (SEQ ID NO: 254) |
| GAEIVY | (SEQ ID NO: 255) |
| AEIVY | (SEQ ID NO: 256) |
| EIVY | (SEQ ID NO: 257) |
| IVY | |
| TDHGAEIVYK | (SEQ ID NO: 259) |
| DHGAEIVYK | (SEQ ID NO: 260) |
| HGAEIVYK | (SEQ ID NO: 261) |
| GAEIVYK | (SEQ ID NO: 262) |
| AEIVYK | (SEQ ID NO: 263) |
| EIVYK | (SEQ ID NO: 264) |
| IVYK | (SEQ ID NO: 265) |
| DHGAEIVYKS | (SEQ ID NO: 266) |
| HGAEIVYKS | (SEQ ID NO: 267) |
| GAEIVYKS | (SEQ ID NO: 268) |
| AEIVYKS | (SEQ ID NO: 269) |
| EIVYKS | (SEQ ID NO: 270) |
| IVYKS | (SEQ ID NO: 271) |
| VYKS | (SEQ ID NO: 272) |
| YKS | |
| HGAEIVYKSP | (SEQ ID NO: 274) |
| GAEIVYKSP | (SEQ ID NO: 275) |
| AEIVYKSP | (SEQ ID NO: 276) |
| EIVYKSP | (SEQ ID NO: 277) |
| IVYKSP | (SEQ ID NO: 278) |
| VYKSP | (SEQ ID NO: 279) |
| YKSP | (SEQ ID NO: 280) |
| KSP | |
| GAEIVYKSPV | (SEQ ID NO: 282) |
| AEIVYKSPV | (SEQ ID NO: 283) |
| EIVYKSPV | (SEQ ID NO: 284) |
| IVYKSPV | (SEQ ID NO: 285) |
| VYKSPV | (SEQ ID NO: 286) |
| YKSPV | (SEQ ID NO: 287) |
| KSPV | (SEQ ID NO: 288) |
| SPV | |
| AEIVYKSPVV | (SEQ ID NO: 290) |
| EIVYKSPVV | (SEQ ID NO: 291) |
| IVYKSPVV | (SEQ ID NO: 292) |
| VYKSPVV | (SEQ ID NO: 293) |
| YKSPVV | (SEQ ID NO: 294) |
| KSPVV | (SEQ ID NO: 295) |
| SPVV | (SEQ ID NO: 296) |
| PVV | |
| EIVYKSPVVS | (SEQ ID NO: 298) |
| IVYKSPVVS | (SEQ ID NO: 299) |
| VYKSPVVS | (SEQ ID NO: 300) |
| YKSPVVS | (SEQ ID NO: 301) |
| KSPVVS | (SEQ ID NO: 302) |
| SPVVS | (SEQ ID NO: 303) |
| PVVS | (SEQ ID NO: 304) |
| VVS | |
| IVYKSPVVSG | (SEQ ID NO: 306) |
| VYKSPVVSG | (SEQ ID NO: 307) |
| YKSPVVSG | (SEQ ID NO: 308) |
| KSPVVSG | (SEQ ID NO: 309) |
| SPVVSG | (SEQ ID NO: 310) |
| PVVSG | (SEQ ID NO: 311) |
| VVSG | (SEQ ID NO: 312) |
| VSG | |
| VYKSPVVSGD | (SEQ ID NO: 314) |
| YKSPVVSGD | (SEQ ID NO: 315) |
| KSPVVSGD | (SEQ ID NO: 316) |
| SPVVSGD | (SEQ ID NO: 317) |
| PVVSGD | (SEQ ID NO: 318) |
| VVSGD | (SEQ ID NO: 319) |
| VSGD | (SEQ ID NO: 320) |
| SGD | |
| YKSPVVSGDT | (SEQ ID NO: 322) |
| KSPVVSGDT | (SEQ ID NO: 323) |
| SPVVSGDT | (SEQ ID NO: 324) |
| PVVSGDT | (SEQ ID NO: 325) |
| VVSGDT | (SEQ ID NO: 326) |
| VSGDT | (SEQ ID NO: 327) |
| SGDT | (SEQ ID NO: 328) |
| GDT | |
| KSPVVSGDTS | (SEQ ID NO: 330) |
| SPVVSGDTS | (SEQ ID NO: 331) |
| PVVSGDTS | (SEQ ID NO: 332) |
| VVSGDTS | (SEQ ID NO: 333) |
| VSGDTS | (SEQ ID NO: 334) |
| SGDTS | (SEQ ID NO: 335) |
| GDTS | (SEQ ID NO: 336) |
| DTS | |
| SPVVSGDTSP | (SEQ ID NO: 338) |
| PVVSGDTSP | (SEQ ID NO: 339) |
| VVSGDTSP | (SEQ ID NO: 340) |
| VSGDTSP | (SEQ ID NO: 341) |
| SGDTSP | (SEQ ID NO: 342) |
| GDTSP | (SEQ ID NO: 343) |
| DTSP | (SEQ ID NO: 344) |
| TSP | |
| PVVSGDTSPR | (SEQ ID NO: 346) |
| VVSGDTSPR | (SEQ ID NO: 347) |
| VSGDTSPR | (SEQ ID NO: 348) |
| SGDTSPR | (SEO ID NO: 349) |
| GDTSPR | (SEQ ID NO: 350) |
| DTSPR | (SEQ ID NO: 351) |
| TSPR | (SEQ ID NO: 352) |
| SPR | |
| HQPGGGKVQI | (SEQ ID NO: 354) |
| QPGGGKVQI | (SEQ ID NO: 355) |
| PGGGKVQI | (SEQ ID NO: 356) |
| GGGKVQI | (SEQ ID NO: 357) |
| GGKVQI | (SEQ ID NO: 358) |
| GKVQI | (SEQ ID NO: 359) |
| KVQI | (SEQ ID NO: 360) |
| VQI | |
| QPGGGKVQII | (SEQ ID NO: 362) |
| PGGGKVQII | (SEQ ID NO: 363) |
| GGGKVQII | (SEQ ID NO: 365) |
| GGKVQII | (SEQ ID NO: 366) |
| GKVQII | (SEQ ID NO: 367) |
| KVQII | (SEQ ID NO: 368) |
| VQII | (SEQ ID NO: 369) |
| QII | |
| PGGGKVQIIN | (SEQ ID NO: 371) |
| GGGKVQIIN | (SEQ ID NO: 372) |
| GGKVQIIN | (SEQ ID NO: 373) |
| GKVQIIN | (SEQ ID NO: 374) |
| KVQIIN | (SEQ ID NO: 375) |
| VQIIN | (SEQ ID NO: 376) |
| QIIN | (SEQ ID NO: 377) |
| IIN | |
| GGGKVQIINK | (SEQ ID NO: 379) |
| GGKVQIINK | (SEQ ID NO: 380) |
| GKVQIINK | (SEQ ID NO: 381) |
| KVQIINK | (SEQ ID NO: 382) |
| IINK | (SEQ ID NO: 383) |
| INK | |
| GGKVQIINKK | (SEQ ID NO: 385) |
| GKVQIINKK | (SEQ ID NO: 386) |
| KVQIINKK | (SEQ ID NO: 387) |
| IINKK | (SEQ ID NO: 388) |
| INKK | (SEQ ID NO: 389) |
| NKK | |
| GKVQIINKKL | (SEQ ID NO: 391) |
| KVQIINKKL | (SEQ ID NO: 392) |
| IINKKL | (SEQ ID NO: 393) |
| INKKL | (SEQ ID NO: 394) |
| NKKL | (SEQ ID NO: 395) |
| KKL | |
| KVQIINKKLD | (SEQ ID NO: 397) |
| VQIINKKLD | (SEQ ID NO: 398) |
| QIINKKLD | (SEQ ID NO: 399) |
| IINKKLD | (SEQ ID NO: 400) |
| INKKLD | (SEQ ID NO: 401) |
| NKKLD | (SEQ ID NO: 402) |
| KKLD | (SEQ ID NO: 403) |
| KLD | |
| VQIINKKLDL | (SEQ ID NO: 405) |
| QIINKKLDL | (SEQ ID NO: 406) |
| IINKKLDL | (SEQ ID NO: 407) |
| INKKLDL | (SEQ ID NO: 408) |
| NKKLDL | (SEQ ID NO: 409) |
| KKLDL | (SEQ ID NO: 410) |
| KLDL | (SEQ ID NO: 411) |
| LDL | |
| QIINKKLDLS | (SEQ ID NO: 413) |
| IINKKLDLS | (SEQ ID NO: 414) |
| INKKLDLS | (SEQ ID NO: 415) |
| NKKLDLS | (SEQ ID NO: 416) |
| KKLDLS | (SEQ ID NO: 417) |
| KLDLS | (SEQ ID NO: 418) |
| LDLS | (SEQ ID NO: 419) |
| IINKKLDLSN | (SEQ ID NO: 420) |
| INKKLDLSN | (SEQ ID NO: 421) |
| NKKLDLSN | (SEQ ID NO: 422) |
| KKLDLSN | (SEQ ID NO: 423) |
| KLDLSN | (SEQ ID NO: 424) |
| LDLSN | (SEQ ID NO: 425) |
| DLSN | (SEQ ID NO: 426) |
| LSN | |
| INKKLDLSNV | (SEQ ID NO: 428) |
| NKKLDLSNV | (SEQ ID NO: 429) |
| KKLDLSNV | (SEQ ID NO: 430) |
| KLDLSNV | (SEQ ID NO: 431) |
| LDLSNV | (SEQ ID NO: 432) |
| DLSNV | (SEQ ID NO: 433) |
| LSNV | (SEQ ID NO: 434) |
| SNV | |
| NKKLDLSNVQ | (SEQ ID NO: 436) |
| KKLDLSNVQ | (SEQ ID NO: 437) |
| KLDLSNVQ | (SEQ ID NO: 438) |
| LDLSNVQ | (SEQ ID NO: 439) |
| DLSNVQ | (SEQ ID NO: 440) |
| LSNVQ | (SEQ ID NO: 441) |
| SNVQ | (SEQ ID NO: 442) |
| NVQ | |
| KKLDLSNVQS | (SEQ ID NO: 444) |
| KLDLSNVQS | (SEQ ID NO: 445) |
| LDLSNVQS | (SEQ ID NO: 446) |
| DLSNVQS | (SEQ ID NO: 447) |
| LSNVQS | (SEQ ID NO: 448) |
| SNVQS | (SEQ ID NO: 449) |
| NVQS | (SEQ ID NO: 450) |
| VOS | |
| SKCGSKDNIK | (SEQ ID NO: 452) |
| KCGSKDNIK | (SEQ ID NO: 453) |
| CGSKDNIK | (SEQ ID NO: 454) |
| GSKDNIK | (SEQ ID NO: 455) |
| SKDNIK | (SEQ ID NO: 456) |
| KDNIK | (SEQ ID NO: 457) |
| DNIK | (SEQ ID NO: 458) |
| NIK | |
| KCGSKDNIKH | (SEQ ID NO: 460) |
| CGSKDNIKH | (SEQ ID NO: 461) |
| GSKDNIKH | (SEQ ID NO: 462) |
| SKDNIKH | (SEQ ID NO: 463) |
| KDNIKH | (SEQ ID NO: 464) |
| DNIKH | (SEQ ID NO: 465) |
| NIKH | (SEQ ID NO: 466) |
| IKH | |
| CGSKDNIKHV | (SEQ ID NO: 468) |
| GSKDNIKHV | (SEQ ID NO: 469) |
| SKDNIKHV | (SEQ ID NO: 470) |
| KDNIKHV | (SEQ ID NO: 471) |
| DNIKHV | (SEQ ID NO: 472) |
| NIKHV | (SEQ ID NO: 473) |
| IKHV | (SEQ ID NO: 474) |
| KHV | |
| GSKDNIKHVP | (SEQ ID NO: 476) |
| SKDNIKHVP | (SEQ ID NO: 477) |
| KDNIKHVP | (SEQ ID NO: 478) |
| DNIKHVP | (SEQ ID NO: 479) |
| IKHVP | (SEQ ID NO: 480) |
| KHVP | (SEQ ID NO: 481) |
| HVP | |
| SKDNIKHVPG | (SEQ ID NO: 483) |
| KDNIKHVPG | (SEQ ID NO: 484) |
| DNIKHVPG | (SEQ ID NO: 485) |
| NIKHVPG | (SEQ ID NO: 486) |
| IKHVPG | (SEQ ID NO: 487) |
| KHVPG | (SEQ ID NO: 488) |
| HVPG | (SEQ ID NO: 489) |
| VPG | |
| KDNIKHVPGG | (SEQ ID NO: 491) |
| DNIKHVPGG | (SEQ ID NO: 492) |
| NIKHVPGG | (SEQ ID NO: 493) |
| IKHVPGG | (SEQ ID NO: 494) |
| KHVPGG | (SEQ ID NO: 495) |
| VPGG | (SEQ ID NO: 496) |
| PGG | |
| DNIKHVPGGG | (SEQ ID NO: 498) |
| NIKHVPGGG | (SEQ ID NO: 499) |
| IKHVPGGG | (SEQ ID NO: 500) |
| VPGGG | (SEQ ID NO: 501) |
| PGGG | (SEQ ID NO: 502) |
| GGG | |
| NIKHVPGGGS | (SEQ ID NO: 504) |
| IKHVPGGGS | (SEQ ID NO: 505) |
| KHVPGGGS | (SEQ ID NO: 506) |
| HVPGGGS | (SEQ ID NO: 507) |
| VPGGGS | (SEQ ID NO: 508) |
| PGGGS | (SEQ ID NO: 509) |
| GGGS | (SEQ ID NO: 510) |
| GGS | |
| IKHVPGGGSV | (SEQ ID NO: 512) |
| KHVPGGGSV | (SEQ ID NO: 513) |
| HVPGGGSV | (SEQ ID NO: 514) |
| VPGGGSV | (SEQ ID NO: 515) |
| PGGGSV | (SEQ ID NO: 516) |
| GGGSV | (SEQ ID NO: 517) |
| GGSV | (SEQ ID NO: 518) |
| GSV | |
| KHVPGGGSVQ | (SEQ ID NO: 520) |
| HVPGGGSVQ | (SEQ ID NO: 521) |
| VPGGGSVQ | (SEQ ID NO: 522) |
| PGGGSVQ | (SEQ ID NO: 523) |
| GGGSVQ | (SEQ ID NO: 524) |
| GGSVQ | (SEQ ID NO: 525) |
| GSVQ | (SEQ ID NO: 526) |
| SVQ | |
| HVPGGGSVQI | (SEQ ID NO: 528) |
| VPGGGSVQI | (SEQ ID NO: 529) |
| PGGGSVQI | (SEQ ID NO: 530) |
| GGGSVQI | (SEQ ID NO: 531) |
| GGSVQI | (SEQ ID NO: 532) |
| GSVQI | (SEQ ID NO: 533) |
| SVQI | (SEQ ID NO: 534) |
| GGSVQIVYKS | (SEQ ID NO: 535) |
| GSVQIVYKS | (SEQ ID NO: 536) |
| SVQIVYKS | (SEQ ID NO: 537) |
| VQIVYKS | (SEQ ID NO: 538) |
| QIVYKS | (SEQ ID NO: 539) |
| IVYKS | (SEQ ID NO: 540) |
| VYKS | (SEQ ID NO: 541) |
| YKS | |
| GSVQIVYKSV | (SEQ ID NO: 543) |
| SVQIVYKSV | (SEQ ID NO: 544) |
| VQIVYKSV | (SEQ ID NO: 545) |
| QIVYKSV | (SEQ ID NO: 546) |
| IVYKSV | (SEQ ID NO: 547) |
| VYKSV | (SEQ ID NO: 548) |
| YKSV | (SEQ ID NO: 549) |
| SVQIVYKSVD | (SEQ ID NO: 550) |
| VQIVYKSVD | (SEQ ID NO: 551) |
| QIVYKSVD | (SEQ ID NO: 552) |
| IVYKSVD | (SEQ ID NO: 553) |
| VYKSVD | (SEQ ID NO: 554) |
| YKSVD | (SEQ ID NO: 555) |
| KSVD | (SEQ ID NO: 556) |
| SVD | |
| VQIVYKSVDL | (SEQ ID NO: 558) |
| QIVYKSVDL | (SEQ ID NO: 559) |
| IVYKSVDL | (SEQ ID NO: 560) |
| VYKSVDL | (SEQ ID NO: 561) |
| YKSVDL | (SEQ ID NO: 562) |
| KSVDL | (SEQ ID NO: 563) |
| SVDL | (SEQ ID NO: 564) |
| QIVYKSVDLS | (SEQ ID NO: 565) |
| IVYKSVDLS | (SEQ ID NO: 566) |
| VYKSVDLS | (SEQ ID NO: 567) |
| YKSVDLS | (SEQ ID NO: 568) |
| KSVDLS | (SEQ ID NO: 569) |
| SVDLS | (SEQ ID NO: 570) |
| IVYKSVDLSK | (SEQ ID NO: 571) |
| VYKSVDLSK | (SEQ ID NO: 572) |
| YKSVDLSK | (SEQ ID NO: 573) |
| KSVDLSK | (SEQ ID NO: 574) |
| SVDLSK | (SEQ ID NO: 364) |
| VYKSVDLSKV | (SEQ ID NO: 575) |
| YKSVDLSKV | (SEQ ID NO: 576) |
| KSVDLSKV | (SEQ ID NO: 577) |
| SVDLSKV | (SEQ ID NO: 578) |
| YKSVDLSKVT | (SEQ ID NO: 579) |
| KSVDLSKVT | (SEQ ID NO: 580) |
| SVDLSKVT | (SEQ ID NO: 581) |
| DLSKVT | (SEQ ID NO: 582) |
| LSKVT | (SEQ ID NO: 583) |
| SKVT | (SEQ ID NO: 584) |
| KVT | |
| HGAEIVYKSV | (SEQ ID NO: 586) |
| GAEIVYKSV | (SEQ ID NO: 587) |
| AEIVYKSV | (SEQ ID NO: 588) |
| GAEIVYKSVV | (SEQ ID NO: 589) |
| AEIVYKSVV | (SEQ ID NO: 590) |
| EIVYKSVV | (SEQ ID NO: 591) |
| IVYKSVV | (SEQ ID NO: 592) |
| VYKSVV | (SEQ ID NO: 593) |
| YKSVV | (SEQ ID NO: 594) |
| KSVV | (SEQ ID NO: 595) |
| SVV | |
| AEIVYKSVVS | (SEQ ID NO: 597) |
| EIVYKSVVS | (SEQ ID NO: 598) |
| IVYKSVVS | (SEQ ID NO: 599) |
| VYKSVVS | (SEQ ID NO: 600) |
| YKSVVS | (SEQ ID NO: 601) |
| KSVVS | (SEQ ID NO: 602) |
| SVVS | (SEQ ID NO: 603) |
| EIVYKSVVSG | (SEQ ID NO: 604) |
| IVYKSVVSG | (SEQ ID NO: 605) |
| VYKSVVSG | (SEQ ID NO: 606) |
| YKSVVSG | (SEQ ID NO: 607) |
| KSVVSG | (SEQ ID NO: 608) |
| SVVSG | (SEQ ID NO: 609) |
| VVSG | (SEQ ID NO: 610) |
| VSG | |
| IVYKSVVSGD | (SEQ ID NO: 612) |
| VYKSVVSGD | (SEQ ID NO: 613) |
| YKSVVSGD | (SEQ ID NO: 614) |
| KSVVSGD | (SEQ ID NO: 615) |
| SVVSGD | (SEQ ID NO: 616) |
| VVSGD | (SEQ ID NO: 617) |
| VYKSVVSGDT | (SEQ ID NO: 618) |
| YKSVVSGDT | (SEQ ID NO: 619) |
| KSVVSGDT | (SEQ ID NO: 620) |
| SVVSGDT | (SEQ ID NO: 621) |
| YKSVVSGDTS | (SEQ ID NO: 622) |
| KSVVSGDTS | (SEQ ID NO: 623) |
| SVVSGDTS | (SEQ ID NO: 624) |
| YKSVVSGDTS | (SEQ ID NO: 625) |
| KSVVSGDTS | (SEQ ID NO: 626) |
| SVVSGDTS | (SEQ ID NO: 627) |
| VVSGDTS | (SEQ ID NO: 628) |
| KSVVSGDTSP | (SEQ ID NO: 629) |
| SVVSGDTSP | (SEQ ID NO: 630) |
| SVVSGDTSPR | (SEQ ID NO: 631) |
| DHGAEIVYKP | (SEQ ID NO: 632) |
| HGAEIVYKP | (SEQ ID NO: 633) |
| GAEIVYKP | (SEQ ID NO: 634) |
| AEIVYKP | (SEQ ID NO: 635) |
| HGAEIVYKPV | (SEQ ID NO: 636) |
| GAEIVYKPV | (SEQ ID NO: 637) |
| AEIVYKPV | (SEQ ID NO: 638) |
| GAEIVYKPVV | (SEQ ID NO: 639) |
| AEIVYKPVV | (SEQ ID NO: 640) |
| EIVYKPVV | (SEQ ID NO: 641) |
| IVYKPVV | (SEQ ID NO: 642) |
| VYKPVV | (SEQ ID NO: 643) |
| YKPVV | (SEQ ID NO: 644) |
| KPVV | (SEQ ID NO: 645) |
| AEIVYKPVVS | (SEQ ID NO: 646) |
| EIVYKPVVS | (SEQ ID NO: 647) |
| IVYKPVVS | (SEQ ID NO: 648) |
| VYKPVVS | (SEQ ID NO: 649) |
| YKPVVS | (SEQ ID NO: 650) |
| KPVVS | (SEQ ID NO: 651) |
| EIVYKPVVSG | (SEQ ID NO: 652) |
| IVYKPVVSG | (SEQ ID NO: 653) |
| VYKPVVSG | (SEQ ID NO: 654) |
| YKPVVSG | (SEQ ID NO: 655) |
| KPVVSG | (SEQ ID NO: 656) |
| IVYKPVVSGD | (SEQ ID NO: 657) |
| VYKPVVSGD | (SEQ ID NO: 658) |
| YKPVVSGD | (SEQ ID NO: 659) |
| KPVVSGD | (SEQ ID NO: 660) |
| VYKPVVSGDT | (SEQ ID NO: 661) |
| YKPVVSGDT | (SEQ ID NO: 662) |
| KPVVSGDT | (SEQ ID NO: 663) |
| YKPVVSGDTS | (SEQ ID NO: 664) |
| KPVVSGDTS | (SEQ ID NO: 665) |
| PVVSGDTS | (SEQ ID NO: 666) |
| VVSGDTS | (SEQ ID NO: 667) |
| KPVVSGDTSP | (SEQ ID NO: 668) |
| CNIK | (SEQ ID NO: 669) |
| CNIKH | (SEQ ID NO: 670) |
| CNIKHV | (SEQ ID NO: 671) |
| CNIKHVPGG | (SEQ ID NO: 672) |
| CNIKHVPGGG | (SEQ ID NO: 673) |
| CNIKHVPGGGS | (SEQ ID NO: 674) |
| ENLKHQPGGG | (SEQ ID NO: 675) |
| NLKHQPGGG | (SEQ ID NO: 676) |
| LKHQPGGG | (SEQ ID NO: 677) |
| KHQPGGG | (SEQ ID NO: 678) |
| HQPGGG | (SEQ ID NO: 679) |
| QPGGG | (SEQ ID NO: 680) |
| TENLKHQPGG | (SEQ ID NO: 681) |
| ENLKHQPGG | (SEQ ID NO: 682) |
| NLKHQPGG | (SEQ ID NO: 683) |
| LKHQPGG | (SEQ ID NO: 684) |
| KHQPGG | (SEQ ID NO: 685) |
| HQPGG | (SEQ ID NO: 686) |
| QPGG | (SEQ ID NO: 687) |
| TENLKHQPG | (SEQ ID NO: 688) |
| ENLKHQPG | (SEQ ID NO: 689) |
| NLKHQPG | (SEQ ID NO: 690) |
| LKHQPG | (SEQ ID NO: 691) |
| KHQPG | (SEQ ID NO: 692) |
| HQPG | (SEQ ID NO: 693) |
| QPG | |
| TENLKHQP | (SEQ ID NO: 695) |
| ENLKHQP | (SEQ ID NO: 696) |
| NLKHQP | (SEQ ID NO: 697) |
| LKHQP | (SEQ ID NO: 698) |
| KHQP | (SEQ ID NO: 699) |
| HQP | |
| TENLKHQ | (SEQ ID NO: 701) |
| ENLKHQ | (SEQ ID NO: 702) |
| NLKHQ | (SEQ ID NO: 703) |
| LKHQ | (SEQ ID NO: 704) |
| KHQ | |
| TENLKH | (SEQ ID NO: 706) |
| ENLKH | (SEQ ID NO: 707) |
| NLKH | (SEQ ID NO: 708) |
| LKH | |
| TENLK | (SEQ ID NO: 710) |
| ENLK | (SEQ ID NO: 711) |
| NLK | |
| TENI | (SEQ ID NO: 713) |
| ENL | |
| TEN | |
| KDNIKHVPGGG | (SEQ ID NO: 716) |
| KDNI | (SEQ ID NO: 717) |
| KDN | |
| IKHVGGG | (SEQ ID NO: 719) |
| IKHVGG | (SEQ ID NO: 720) |
| IKHVG | (SEQ ID NO: 721) |
| KHVGGG | (SEQ ID NO: 722) |
| KHVGG | (SEQ ID NO: 723) |
| KHVG | (SEQ ID NO: 724) |
| LGNIHHKPGGG | (SEQ ID NO: 725) |
| GNIHHKPGGG | (SEQ ID NO: 726) |
| NIHHKPGGG | (SEQ ID NO: 727) |
| IHHKPGGG | (SEQ ID NO: 728) |
| HHKPGGG | (SEQ ID NO: 729) |
| KPGGG | (SEQ ID NO: 730) |
| LGNIHHKPGG | (SEQ ID NO: 731) |
| GNIHHKPGG | (SEQ ID NO: 732) |
| NIHHKPGG | (SEQ ID NO: 733) |
| IHHKPGG | (SEQ ID NO: 734) |
| HHKPGG | (SEQ ID NO: 735) |
| KPGG | (SEQ ID NO: 736) |
| LGNIHHKPG | (SEQ ID NO: 737) |
| GNIHHKPG | (SEQ ID NO: 738) |
| NIHHKPG | (SEQ ID NO: 739) |
| IHHKPG | (SEQ ID NO: 740) |
| HHKPG | (SEQ ID NO: 741) |
| HKPG | (SEQ ID NO: 742) |
| KPG | |
| LGNIHHKP | (SEQ ID NO: 744) |
| GNIHHKP | (SEQ ID NO: 745) |
| NIHHKP | (SEQ ID NO: 746) |
| IHHKP | (SEQ ID NO: 747) |
| HHKP | (SEQ ID NO: 748) |
| HKP | |
| LGNIHHK | (SEQ ID NO: 750) |
| GNIHHK | (SEQ ID NO: 751) |
| NIHHK | (SEQ ID NO: 752) |
| IHHK | (SEQ ID NO: 753) |
| HHK | |
| LGNIHH | (SEQ ID NO: 755) |
| GNIHH | (SEQ ID NO: 756) |
| NIHH | (SEQ ID NO: 757) |
| IHH | |
| LGNIH | (SEQ ID NO: 759) |
| GNIH | (SEQ ID NO: 760) |
| NIH | |
| LGNI | (SEQ ID NO: 762) |
| GNI | |
| LGN | |
| LDNITHVPGGG | (SEQ ID NO: 765) |
| DNITHVPGGG | (SEQ ID NO: 766) |
| NITHVPGGG | (SEQ ID NO: 767) |
| ITHVPGGG | (SEQ ID NO: 768) |
| THVPGGG | (SEQ ID NO: 769) |
| LDNITHVPGG | (SEQ ID NO: 770) |
| DNITHVPGG | (SEQ ID NO: 771) |
| NITHVPGG | (SEQ ID NO: 772) |
| ITHVPGG | (SEQ ID NO: 773) |
| THVPGG | (SEQ ID NO: 774) |
| LDNITHVPG | (SEQ ID NO: 775) |
| DNITHVPG | (SEQ ID NO: 776) |
| NITHVPG | (SEQ ID NO: 777) |
| ITHVPG | (SEQ ID NO: 778) |
| THVPG | (SEQ ID NO: 779) |
| LDNITHVP | (SEQ ID NO: 780) |
| DNITHVP | (SEQ ID NO: 781) |
| NITHVP | (SEQ ID NO: 782) |
| ITHVP | (SEQ ID NO: 783) |
| THVP | (SEQ ID NO: 784) |
| LDNITHV | (SEQ ID NO: 785) |
| DNITHV | (SEQ ID NO: 786) |
| NITHV | (SEQ ID NO: 787) |
| ITHV | (SEQ ID NO: 788) |
| THV | |
| LDNITH | (SEQ ID NO: 790) |
| DNITH | (SEQ ID NO: 791) |
| NITH | (SEQ ID NO: 792) |
| ITH | |
| LDNIT | (SEQ ID NO: 794) |
| DNIT | (SEQ ID NO: 795) |
| NIT | |
| LDNI | (SEQ ID NO: 797) |
| LDN | |
| KNVKSKIGST | (SEQ ID NO: 799) |
| NVKSKIGST | (SEQ ID NO: 800) |
| VKSKIGST | (SEQ ID NO: 801) |
| KSKIGST | (SEQ ID NO: 802) |
| SKIGST | (SEQ ID NO: 803) |
| KIGST | (SEQ ID NO: 804) |
| IGST | (SEQ ID NO: 805) |
| GST | |
| NVKSKIGSTE | (SEQ ID NO: 807) |
| VKSKIGSTE | (SEQ ID NO: 808) |
| KSKIGSTE | (SEQ ID NO: 809) |
| SKIGSTE | (SEQ ID NO: 810) |
| KIGSTE | (SEQ ID NO: 811) |
| IGSTE | (SEQ ID NO: 812) |
| GSTE | (SEQ ID NO: 813) |
| STE | |
| VKSKIGSTEN | (SEQ ID NO: 815) |
| KSKIGSTEN | (SEQ ID NO: 816) |
| SKIGSTEN | (SEQ ID NO: 817) |
| KIGSTEN | (SEQ ID NO: 818) |
| IGSTEN | (SEQ ID NO: 819) |
| GSTEN | (SEQ ID NO: 820) |
| STEN | (SEQ ID NO: 821) |
| KSKIGSTENL | (SEQ ID NO: 822) |
| SKIGSTENL | (SEQ ID NO: 823) |
| KIGSTENL | (SEQ ID NO: 824) |
| IGSTENL | (SEQ ID NO: 825) |
| GSTENL | (SEQ ID NO: 826) |
| STENL | (SEQ ID NO: 827) |
| SKIGSTENLK | (SEQ ID NO: 828) |
| KIGSTENLK | (SEQ ID NO: 829) |
| IGSTENLK | (SEQ ID NO: 830) |
| GSTENLK | (SEQ ID NO: 831) |
| STENLK | (SEQ ID NO: 832) |
| KIGSTENLKH | (SEQ ID NO: 833) |
| IGSTENLKH | (SEQ ID NO: 834) |
| GSTENLKH | (SEQ ID NO: 835) |
| STENLKH | (SEQ ID NO: 836) |
| IGSTENLKHQ | (SEQ ID NO: 837) |
| GSTENLKHQ | (SEQ ID NO: 838) |
| STENLKHQ | (SEQ ID NO: 839) |
| GSTENLKHQP | (SEQ ID NO: 840) |
| STENLKHQP | (SEQ ID NO: 841) |
| STENLKHQPG | (SEQ ID NO: 842) |
| SNVQSKCGSK | (SEQ ID NO: 843) |
| NVQSKCGSK | (SEQ ID NO: 844) |
| VQSKCGSK | (SEQ ID NO: 845) |
| QSKCGSK | (SEQ ID NO: 846) |
| SKCGSK | (SEQ ID NO: 847) |
| KCGSK | (SEQ ID NO: 848) |
| CGSK | (SEQ ID NO: 849) |
| GSK | |
| NVQSKCGSKD | (SEQ ID NO: 851) |
| VQSKCGSKD | (SEQ ID NO: 852) |
| QSKCGSKD | (SEQ ID NO: 853) |
| SKCGSKD | (SEQ ID NO: 854) |
| KCGSKD | (SEQ ID NO: 855) |
| CGSKD | (SEQ ID NO: 856) |
| GSKD | (SEQ ID NO: 857) |
| SKD | |
| VQSKCGSKDN | (SEQ ID NO: 859) |
| QSKCGSKDN | (SEQ ID NO: 860) |
| SKCGSKDN | (SEO ID NO: 861) |
| KCGSKDN | (SEQ ID NO: 862) |
| CGSKDN | (SEQ ID NO: 863) |
| GSKDN | (SEQ ID NO: 864) |
| SKDN | (SEQ ID NO: 865) |
| QSKCGSKDNI | (SEQ ID NO: 866) |
| SKCGSKDNI | (SEQ ID NO: 867) |
| KCGSKDNI | (SEQ ID NO: 868) |
| CGSKDNI | (SEQ ID NO: 869) |
| GSKDNI | (SEQ ID NO: 870) |
| SKDNI | (SEQ ID NO: 871) |
| SKVTSKCGSL | (SEQ ID NO: 872) |
| KVTSKCGSL | (SEQ ID NO: 873) |
| VTSKCGSL | (SEQ ID NO: 874) |
| TSKCGSL | (SEQ ID NO: 875) |
| SKCGSL | (SEQ ID NO: 876) |
| KCGSL | (SEQ ID NO: 877) |
| CGSL | (SEQ ID NO: 878) |
| GSL | |
| KVTSKCGSLG | (SEQ ID NO: 880) |
| VTSKCGSLG | (SEQ ID NO: 881) |
| TSKCGSLG | (SEQ ID NO: 882) |
| SKCGSLG | (SEQ ID NO: 883) |
| KCGSLG | (SEQ ID NO: 884) |
| CGSLG | (SEQ ID NO: 885) |
| GSLG | (SEQ ID NO: 886) |
| SLG | |
| VTSKCGSLGN | (SEQ ID NO: 888) |
| TSKCGSLGN | (SEQ ID NO: 889) |
| SKCGSLGN | (SEQ ID NO: 890) |
| KCGSLGN | (SEQ ID NO: 891) |
| CGSLGN | (SEQ ID NO: 892) |
| GSLGN | (SEQ ID NO: 893) |
| SLGN | (SEQ ID NO: 894) |
| TSKCGSLGNI | (SEQ ID NO: 895) |
| SKCGSLGNI | (SEQ ID NO: 896) |
| KCGSLGNI | (SEQ ID NO: 897) |
| CGSLGNI | (SEQ ID NO: 898) |
| GSLGNI | (SEQ ID NO: 899) |
| SLGNI | (SEQ ID NO: 900) |
| SKCGSLGNIH | (SEQ ID NO: 901) |
| KCGSLGNIH | (SEQ ID NO: 902) |
| CGSLGNIH | (SEQ ID NO: 903) |
| GSLGNIH | (SEQ ID NO: 904) |
| SLGNIH | (SEQ ID NO: 905) |
| KCGSLGNIHH | (SEQ ID NO: 906) |
| CGSLGNIHH | (SEQ ID NO: 907) |
| GSLGNIHH | (SEQ ID NO: 908) |
| SLGNIHH | (SEQ ID NO: 909) |
| CGSLGNIHHK | (SEQ ID NO: 910) |
| GSLGNIHHK | (SEQ ID NO: 911) |
| SLGNIHHK | (SEQ ID NO: 912) |
| GSLGNIHHKP | (SEQ ID NO: 913) |
| SLGNIHHKP | (SEQ ID NO: 914) |
| SLGNIHHKPG | (SEQ ID NO: 915) |
| DRVQSKIGSL | (SEQ ID NO: 916) |
| RVQSKIGSL | (SEQ ID NO: 917) |
| VQSKIGSL | (SEQ ID NO: 918) |
| QSKIGSL | (SEQ ID NO: 919) |
| SKIGSL | (SEQ ID NO: 920) |
| KIGSL | (SEQ ID NO: 921) |
| IGSL | (SEQ ID NO: 922) |
| RVQSKIGSLD | (SEQ ID NO: 923) |
| VQSKIGSLD | (SEQ ID NO: 924) |
| QSKIGSLD | (SEQ ID NO: 925) |
| SKIGSLD | (SEQ ID NO: 926) |
| KIGSLD | (SEQ ID NO: 927) |
| IGSLD | (SEQ ID NO: 928) |
| GSLD | (SEQ ID NO: 929) |
| SLD | |
| VQSKIGSLDN | (SEQ ID NO: 931) |
| QSKIGSLDN | (SEQ ID NO: 932) |
| SKIGSLDN | (SEQ ID NO: 933) |
| KIGSLDN | (SEQ ID NO: 934) |
| IGSLDN | (SEQ ID NO: 935) |
| GSLDN | (SEQ ID NO: 936) |
| SLDN | (SEQ ID NO: 937) |
| QSKIGSLDNI | (SEQ ID NO: 938) |
| SKIGSLDNI | (SEQ ID NO: 939) |
| KIGSLDNI | (SEQ ID NO: 940) |
| IGSLDNI | (SEQ ID NO: 941) |
| GSLDNI | (SEQ ID NO: 942) |
| SKIGSLDNIT | (SEQ ID NO: 943) |
| KIGSLDNIT | (SEQ ID NO: 944) |
| IGSLDNIT | (SEQ ID NO: 945) |
| GSLDNIT | (SEQ ID NO: 946) |
| SLDNIT | (SEQ ID NO: 947) |
| KIGSLDNITH | (SEQ ID NO: 948) |
| IGSLDNITH | (SEQ ID NO: 949) |
| GSLDNITH | (SEQ ID NO: 950) |
| SLDNITH | (SEQ ID NO: 951) |
| IGSLDNITHV | (SEQ ID NO: 952) |
| GSLDNITHV | (SEQ ID NO: 953) |
| SLDNITHV | (SEQ ID NO: 954) |
| GSLDNITHVP | (SEQ ID NO: 955) |
| SLDNITHVP | (SEQ ID NO: 956) |
| SLDNITHVPG | (SEQ ID NO: 957) |
| PDLKNVKS | (SEQ ID NO: 958) |
| DLKNVKSK | (SEQ ID NO: 959) |
| LKNVKSKI | (SEQ ID NO: 960) |
| KNVKSKIG | (SEQ ID NO: 961) |
| NVKSKIGS | (SEQ ID NO: 962) |
| DLSNVQSK | (SEQ ID NO: 963) |
| LSNVQSKC | (SEQ ID NO: 964) |
| SNVQSKCG | (SEQ ID NO: 965) |
| NVQSKCGS | (SEQ ID NO: 966) |
| VDLSKVTS | (SEQ ID NO: 967) |
| DLSKVTSK | (SEQ ID NO: 968) |
| LSKVTSKC | (SEQ ID NO: 969) |
| SKVTSKCG | (SEQ ID NO: 970) |
| KVTSKCGS | (SEQ ID NO: 971) |
| LDFKDRVQ | (SEQ ID NO: 972) |
| DFKDRVQS | (SEQ ID NO: 973) |
| FKDRVQSK | (SEQ ID NO: 974) |
| KDRVQSKI | (SEQ ID NO: 975) |
| DRVQSKIG | (SEQ ID NO: 976) |
| RVQSKIGS | (SEQ ID NO: 977) |
| SKIGSTENLKH | (SEQ ID NO: 978) |
| SKIGSTENIKH | (SEQ ID NO: 979) |
| SKIGSKDNLKH | (SEQ ID NO: 980) |
| SKIGSKENIKH | (SEQ ID NO: 981) |
| SKIGSLENLKH | (SEQ ID NO: 982) |
| SKIGSLENIKH | (SEQ ID NO: 983) |
| SKIGSTDNLKH | (SEQ ID NO: 984) |
| SKIGSTDNIKH | (SEQ ID NO: 985) |
| SKIGSKDNIKH | (SEQ ID NO: 986) |
| SKIGSLDNLKH | (SEQ ID NO: 987) |
| SKIGSLDNIKH | (SEQ ID NO: 988) |
| SKIGSTGNLKH | (SEQ ID NO: 989) |
| SKIGSTGNIKH | (SEQ ID NO: 990) |
| SKIGSKGNLKH | (SEQ ID NO: 991) |
| SKIGSKGNIKH | (SEQ ID NO: 992) |
| SKIGSLGNLKH | (SEQ ID NO: 993) |
| SKIGSLGNIKH | (SEQ ID NO: 994) |

Lys Xaa₁ Xaa₂ Ser Xaa₃ Xaa₄ Asn Xaa₅ Xaa₆ His (SEQ ID NO: 995), wherein
Xaa₁ is I or C;
Xaa₂ is G;
Xaa₃ is T, K or L;
Xaa₄ is E, D or G;
Xaa₅ is L or I;
Xaa₆ is K, H or T.

(SEQ ID NO: 996)
(Q/E)IVYK(S/P)
(SEQ ID NO: 57)
DAEFRHDRRQIVYKPV
(SEQ ID NO: 58)
DAEFRHDRREIVYKSV
(SEQ ID NO: 59)
DAEFRHDRRQIVYKPVGGC
(SEQ ID NO: 60)
DAEFRHDRRQIVYKPVC
(SEQ ID NO: 61)
DAEFRHDRRQIVYKPVAAC
(SEQ ID NO: 62)
DAEFRHDRRQIVYKPVKKC
(SEQ ID NO: 63)
DAEFRHDRREIVYKSPGGC
(SEQ ID NO: 64)
DAEFRHDRREIVYKSPAAC
(SEQ ID NO: 65)
DAEFRHDRREIVYKSPC
(SEQ ID NO: 66)
DAEFRHDRREIVYKSPKKC
(SEQ ID NO: 67)
DAEFRHDSGYEVHHQKLFFAEDVGSNKG
(SEQ ID NO: 68)
GGGSVQIVYKPVDLS
(RVRR; SEQ ID NO: 69)
Arg-Val-Arg-Arg
SEQ ID NO: 80)
Gly-Ala-Gly-Ala
(AGAG; SEQ ID NO: 81)
Ala-Gly-Ala-Gly
(KGKG; SEQ ID NO: 82)
Lys -Gly-Lys-Gly,
(SEQ ID NO: 70)
DAEFRHDRRQIVYKPVXXC,
If present, XX can be GG or AA or KK or SS
(SEQ ID NO: 79)
DAEFRHDRREIVYKSVXXC,
If present, XX can be GG or AA or KK or SS
(SEQ ID NO: 71)
DAEFRHDC
(SEQ ID NO: 72)
QIVYKPVGGC
(SEQ ID NO: 73)
GGSQIVYKPVDLS
(SEQ ID NO: 74)
DAEFRHDRREIVYKSVGGC
(SEQ ID NO: 75)
DAEFRHDRREIVYKSVAAC
(SEQ ID NO: 76)
DAEFRHDRREIVYKSVC
(SEQ ID NO: 77)
DAEFRHDRREIVYKSVKKC
(SEQ ID NO: 78)
DAEFRHDRRNIKHVPGGC
(SEQ ID NO: 997)
DAEFRHDRRVKSKIGSTGGC
(SEQ ID NO: 998)
DAEFRHDRRSKIGSTENGGC
(SEQ ID NO: 999)
DAEFRHDRRTENLKHQPGGC
(SEQ ID NO: 1000)
DAEFRHDRRENLKHQPGGGC
(SEQ ID NO: 1001)
DAEFRHDRRSKIGSKDNIKHGGC
MTBR peptide 1
(SEQ ID NO: 1058)
QTAPVPMPDLKNVKSKIGSTENLKHQPGGGK,
MTBR peptide 2
(SEQ ID NO: 1059)
VQIINKKLDLSNVQSKCGSKDNIKHVPGGGS,
MTBR peptide 3
(SEQ ID NO: 1060)
VQIVYKPVDLSKVTSKCGSLGNIHHKPGGGQ,
MTBR peptide 4
(SEQ ID NO: 1061)
VEVKSEKLDFKDRVOSKIGSLDNITHVPGGGN,
(SEQ ID NO: 1062)
GGGS,
(SEQ ID NO: 1063)
GGGGS,

The disclosure provides peptide compositions and immunotherapy compositions comprising an amyloid-beta (Aβ) peptide and a tau peptide. The disclosure also provides methods of treating or effecting prophylaxis of Alzheimer's disease or other diseases with beta-amyloid deposition in a subject, including methods of clearing deposits, inhibiting or reducing aggregation of Aβ and/or tau, blocking the uptake by neurons, clearing amyloid, and inhibiting propagation of tau seeds in a subject having or at risk of developing Alzheimer's disease or other diseases containing tau and/or amyloid-beta accumulations. The methods include administering to such patients the compositions comprising an amyloid-beta (Aβ) peptide and a tau peptide.

1-89. (canceled)

90. A polypeptide comprising a first peptide comprising 3-10 contiguous amino acids from residues 1-10 of SEQ ID NO:01, wherein the 3-10 contiguous amino acids comprise an amino acid sequence selected from DAE, EFR, FRH, RHD, HDS, DSG, and SGY linked to a second peptide comprising 3-13 contiguous amino acids from residues 258-272 of SEQ ID NO: 02, wherein the 3-13 contiguous amino acids comprise an amino acid sequence selected from SKI, KIG, IGS, GST, STE, TEN, ENL, NLK, LKH, KHQ, HQP, QPG, PGG and GGG.

91. The polypeptide of claim 90, wherein one or both of the 3-10 contiguous amino acids of the first peptide and the 3-13 contiguous amino acids of the second peptide is at least four contiguous amino acids.

92. The polypeptide of claim 90, wherein one or both of the 3-10 contiguous amino acids of the first peptide and the 3-13 contiguous amino acids of the second peptide is at least five contiguous amino acids.

93. The polypeptide of claim 90, wherein one or both of the 3-10 contiguous amino acids of the first peptide and the 3-13 contiguous amino acids of the second peptide is at least six contiguous amino acids.

94. The polypeptide of claim 90, wherein one or both of the 3-10 contiguous amino acids of the first peptide and the 3-13 contiguous amino acids of the second peptide is at least seven contiguous amino acids.

95. The polypeptide of claim 90, wherein:
(a) the 3-10 contiguous amino acids of the first peptide comprises an amino acid sequence selected from
(SEQ ID NO: 03)
DAEFRHDSGY,
(SEQ ID NO: 04)
DAEFRHDSG,
(SEQ ID NO: 05)
DAEFRHDS,
(SEQ ID NO: 06)
DAEFRHD,
(SEQ ID NO: 07)
DAEFRH,
(SEQ ID NO: 08)
DAEFR,
(SEQ ID NO: 09)
DAEF,
(SEQ ID NO: 11)
AEFRHDSGY,
(SEQ ID NO: 12)
AEFRHDSG,
(SEQ ID NO: 13)
AEFRHDS,
(SEQ ID NO: 14)
AEFRHD,
(SEQ ID NO: 15)
AEFRH,
(SEQ ID NO: 16)
AEFR,
(SEQ ID NO: 18)
EFRHDSGY,
(SEQ ID NO: 19)
EFRHDSG,
(SEQ ID NO: 20)
EFRHDS,
(SEQ ID NO: 21)
EFRHD,
(SEQ ID NO: 22)
EFRH,
(SEQ ID NO: 24)
FRHDSGY,
(SEQ ID NO: 25)
FRHDSG,
(SEQ ID NO: 26)
FRHDS,
(SEQ ID NO: 27)
FRHD,
(SEQ ID NO: 29)
RHDSGY,
(SEQ ID NO: 30)
RHDSG,
(SEQ ID NO: 31)
RHDS,
(SEQ ID NO: 33)
HDSGY,
(SEQ ID NO: 34)
HDSG,
and
(SEQ ID NO: 36)
DSGY,
and
(b) the 3-13 contiguous amino acids of the second peptide comprises an amino acid sequence selected from
(SEQ ID NO: 675)
ENLKHQPGGG,
(SEQ ID NO: 676)
NLKHQPGGG,
(SEQ ID NO: 677)
LKHQPGGG,
(SEQ ID NO: 678)
KHQPGGG,
(SEQ ID NO: 679,)
HQPGGG,
(SEQ ID NO: 680)
QPGGG,
(SEQ ID NO: 681)
TENLKHQPGG,
(SEQ ID NO: 682)
ENLKHQPGG,
(SEQ ID NO: 683)
NLKHQPGG,
(SEQ ID NO: 684)
LKHQPGG,
(SEQ ID NO: 685)
KHQPGG,
(SEQ ID NO: 686)
HQPGG,
(SEQ ID NO: 687)
QPGG,
(SEQ ID NO: 688)
TENLKHQPG,
(SEQ ID NO: 689)
ENLKHQPG,
(SEQ ID NO: 690)
NLKHQPG,
(SEQ ID NO: 691)
LKHQPG,
(SEQ ID NO: 692)
KHQPG,
(SEQ ID NO: 693)
HQPG,
(SEQ ID NO: 695)
TENLKHQP,
(SEQ ID NO: 696)
ENLKHQP,
(SEQ ID NO: 697)
NLKHQP,
(SEQ ID NO: 698)
LKHQP,
(SEQ ID NO: 699)
KHQP,
(SEQ ID NO: 701)
TENLKHQ,
(SEQ ID NO: 702)
ENLKHQ,
(SEQ ID NO: 703)
NLKHQ,
(SEQ ID NO: 704)
LKHQ,
(SEQ ID NO: 706,)
TENLKH,
(SEQ ID NO: 707)
ENLKH,
(SEQ ID NO: 708)
NLKH,
(SEQ ID NO: 710)
TENLK,
(SEQ ID NO: 711)
ENLK,
(SEQ ID NO: 713)
TENL,
(SEQ ID NO: 803)
SKIGST,
(SEQ ID NO: 804)
KIGST,
(SEQ ID NO: 805)
IGST,
(SEQ ID NO: 808)
VKSKIGSTE,
(SEQ ID NO: 809)
KSKIGSTE,
(SEQ ID NO: 810)
SKIGSTE,
(SEQ ID NO: 811)
KIGSTE,
(SEQ ID NO: 812)
IGSTE,
(SEQ ID NO: 813)
GSTE,
(SEQ ID NO: 817)
SKIGSTEN,
(SEQ ID NO: 818)
KIGSTEN,
(SEQ ID NO: 819)
IGSTEN,
(SEQ ID NO: 820)
GSTEN,
(SEQ ID NO: 821)
STEN,
(SEQ ID NO: 823)
SKIGSTENL,
(SEQ ID NO: 824)
KIGSTENL,
(SEQ ID NO: 825)
IGSTENL,
(SEQ ID NO: 826)
GSTENL,
(SEQ ID NO: 827)
STENL,
(SEQ ID NO: 828)
SKIGSTENLK,
(SEQ ID NO: 829)
KIGSTENLK,
(SEQ ID NO: 830)
IGSTENLK,
(SEQ ID NO: 831)
GSTENLK,
(SEQ ID NO: 832)
STENLK,
(SEQ ID NO: 833)
KIGSTENLKH,
(SEQ ID NO: 834)
IGSTENLKH,
(SEQ ID NO: 835)
GSTENLKH,
(SEQ ID NO: 836)
STENLKH,
(SEQ ID NO: 837)
IGSTENLKHQ,
(SEQ ID NO: 838)
GSTENLKHQ,
(SEQ ID NO: 839)
STENLKHQ,
(SEQ ID NO: 840)
GSTENLKHQP,
(SEQ ID NO: 841)
STENLKHQP,
(SEQ ID NO: 842)
STENLKHQPG,
and
(SEQ ID NO: 978)
SKIGSTENLKH.

96. The polypeptide of claim 95, wherein the 3-10 contiguous amino acids of the first peptide comprises an amino acid sequence selected from DAEFRHD (SEQ ID NO:06), DAEFR (SEQ ID NO: 08), EFRHD (SEQ ID NO:21).

97. The polypeptide of claim 95, wherein the 3-13 contiguous amino acids of the second peptide comprises an amino acid sequence selected from SKIGSTEN (SEQ ID NO:817), TENLKHQP (SEQ ID NO:695), ENLKHQP (SEQ ID NO:696), and ENLKHQPG (SEQ ID NO:689).

98. The polypeptide of claim 95, wherein the 3-10 contiguous amino acids of the first peptide comprises an amino acid sequence selected from DAEFRHD (SEQ ID NO:06), DAEFR (SEQ ID NO: 08), and EFRHD (SEQ ID NO:21), and the wherein the 3-13 contiguous amino acids of the second peptide comprises an amino acid sequence selected from SKIGSTEN (SEQ ID NO: 817), TENLKHQP (SEQ ID NO:695), ENLKHQP (SEQ ID NO:696), and ENLKHQPG (SEQ ID NO:689).

99. The polypeptide of claim 95, wherein the first peptide and second peptide are linked by a cleavable linker.

100. The polypeptide of claim 99, wherein the cleavable linker comprises an amino acid sequence comprising arginine-arginine (Arg-Arg), arginine-valine-arginine-arginine (Arg-Val-Arg-Arg (SEQ ID NO:69)), valine-citrulline (Val-Cit), valine-arginine (Val-Arg), valine-lysine (Val-Lys), valine-alanine (Val-Ala), phenylalanine-lysine (Phe-Lys), glycine-alanine-glycine-alanine (Gly-Ala-Gly-Ala; SEQ ID NO:80), alanine-glycine-alanine-glycine (Ala-Gly-Ala-Gly; SEQ ID NO:81), or lysine-glycine-lysine-glycine (Lys-Gly-Lys-Gly; SEQ ID NO:82).

101. An immunotherapy composition, comprising the polypeptide of claim 99, a carrier, and a linker to the carrier located at either a C-terminus or a N-terminus of the polypeptide.

102. The immunotherapy composition of claim 101, wherein the linker to the carrier comprises an amino acid sequence selected from the group consisting of GG, GGG, AA, AAA, KK, KKK, SS, SSS, GAGA (SEQ ID NO:80), AGAG (SEQ ID NO:81), and KGKG (SEQ ID NO: 82).

103. The immunotherapy composition of claim 102, wherein the polypeptide, or linker to the carrier, if present, further comprises a C-terminal cysteine (C).

104. The immunotherapy composition of claim 103, wherein the carrier comprises serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

105. A pharmaceutical formulation comprising the polypeptide of claim 1 and at least one adjuvant.

106. The pharmaceutical formulation of claim 105, wherein the adjuvant is selected from the group consisting of aluminum hydroxide, aluminum phosphate, aluminum sulfate, 3 De-O-acylated monophosphoryl lipid A (MPL), QS-21, TQL1055, QS-18, QS-17, QS-7, Complete Freund's Adjuvant (CFA), Incomplete Freund's Adjuvant (IFA), oil in water emulsions (such as squalene or peanut oil), CpG, polyglutamic acid, polylysine, AddaVax™, MF59®, combinations thereof, or liposomal formulation or a multiple antigen presenting system (MAP).

107. A pharmaceutical composition comprising the immunotherapy composition of claim 101 and at least one adjuvant.

108. A nucleic acid comprising a nucleic acid sequence encoding the polypeptide of claim 90.

109. A nucleic acid immunotherapy composition comprising the nucleic acid of claim 108 and at least one adjuvant.

110. An immunization kit comprising the immunotherapy composition of claim 101 and an adjuvant, wherein the immunotherapy composition is in a first container and the adjuvant is in a second container.

111. A immunization kit comprising the nucleic acid immunotherapy composition of claim 109 and an adjuvant, wherein the nucleic acid is in a first container and the adjuvant is in a second container.


SEQUENCE LISTING (1063 sequences)

SEQ ID NO: 1 - protein, 42 aa, synthetic construct [sequence omitted]

SEQ ID NO: 2 - protein, 441 aa, synthetic construct
  REGION 1..441: note = Synthetic peptide
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 10 aa, synthetic construct
  REGION 1..10: note = Synthetic peptide
DAEFRHDSGY

SEQ ID NO: 4 - protein, 9 aa, synthetic construct
  REGION 1..9: note = Synthetic peptide
DAEFRHDSG

SEQ ID NO: 5 - protein, 8 aa, synthetic construct
  REGION 1..8: note = Synthetic peptide
DAEFRHDS

SEQ ID NO: 6 - protein, 7 aa, synthetic construct
  REGION 1..7: note = Synthetic peptide
DAEFRHD

SEQ ID NO: 7 - protein, 6 aa, synthetic construct
  REGION 1..6: note = Synthetic peptide
DAEFRH

SEQ ID NO: 8 - protein, 5 aa, synthetic construct
  REGION 1..5: note = Synthetic peptide
DAEFR

SEQ ID NO: 9 - protein, 4 aa, synthetic construct
  REGION 1..4: note = Synthetic peptide
DAEF

SEQ ID NO: 10 - skipped

SEQ ID NO: 11 - protein, 9 aa, synthetic construct
  REGION 1..9: note = Synthetic peptide
AEFRHDSGY

SEQ ID NO: 12 - protein, 8 aa, synthetic construct
  REGION 1..8: note = Synthetic peptide
AEFRHDSG

SEQ ID NO: 13 - protein, 7 aa, synthetic construct
  REGION 1..7: note = Synthetic peptide
AEFRHDS

SEQ ID NO: 14 - protein, 6 aa, synthetic construct
  REGION 1..6: note = Synthetic peptide
AEFRHD

SEQ ID NO: 15 - protein, 5 aa, synthetic construct
  REGION 1..5: note = Synthetic peptide
AEFRH

SEQ ID NO: 16 - protein, 4 aa, synthetic construct
  REGION 1..4: note = Synthetic peptide
AEFR

SEQ ID NO: 17 - skipped

SEQ ID NO: 18 - protein, 8 aa, synthetic construct
  REGION 1..8: note = Synthetic peptide
EFRHDSGY

SEQ ID NO: 19 - protein, 7 aa, synthetic construct
  REGION 1..7: note = Synthetic peptide
EFRHDSG

SEQ ID NO: 20 - protein, 6 aa, synthetic construct
  REGION 1..6: note = Synthetic peptide
EFRHDS

SEQ ID NO: 21 - protein, 5 aa, synthetic construct
  REGION 1..5: note = Synthetic peptide
EFRHD

SEQ ID NO: 22 - protein, 4 aa, synthetic construct
  REGION 1..4: note = Synthetic peptide
EFRH

SEQ ID NO: 23 - skipped

SEQ ID NO: 24 - protein, 7 aa, synthetic construct
  REGION 1..7: note = Synthetic peptide
FRHDSGY

SEQ ID NO: 25 - protein, 6 aa, synthetic construct
  REGION 1..6: note = Synthetic peptide
FRHDSG

SEQ ID NO: 26 - protein, 5 aa, synthetic construct
  REGION 1..5: note = Synthetic peptide
FRHDS

SEQ ID NO: 27 - protein, 4 aa, synthetic construct
  REGION 1..4: note = Synthetic peptide
FRHD

SEQ ID NO: 28 - skipped

SEQ ID NO: 29 - protein, 6 aa, synthetic construct
  REGION 1..6: note = Synthetic peptide
RHDSGY

SEQ ID NO: 30 - protein, 5 aa, synthetic construct
  REGION 1..5: note = Synthetic peptide
RHDSG

SEQ ID NO: 31 - protein, 4 aa, synthetic construct
  REGION 1..4: note = Synthetic peptide
RHDS

SEQ ID NO: 32 - skipped

SEQ ID NO: 33 - protein, 5 aa, synthetic construct
  REGION 1..5: note = Synthetic peptide
HDSGY

SEQ ID NO: 34 - protein, 4 aa, synthetic construct
  REGION 1..4: note = Synthetic peptide
HDSG

SEQ ID NO: 35 - skipped

SEQ ID NO: 36 - protein, 4 aa, synthetic construct
  REGION 1..4: note = Synthetic peptide
DSGY

SEQ ID NO: 37-38 - skipped (2 entries)

SEQ ID NO: 39 - protein, 7 aa, synthetic construct
  REGION 1..7: note = Synthetic peptide
QIVYKPV

SEQ ID NO: 40 - protein, 6 aa, synthetic construct
  REGION 1..6: note = Synthetic peptide
QIVYKP

SEQ ID NO: 41 - protein, 7 aa, synthetic construct
  REGION 1..7: note = Synthetic peptide
QIVYKSV

SEQ ID NO: 42 - protein, 7 aa, synthetic construct
  REGION 1..7: note = Synthetic peptide
EIVYKSV

SEQ ID NO: 43 - protein, 7 aa, synthetic construct
  REGION 1..7: note = Synthetic peptide
EIVYKSP

SEQ ID NO: 44 - protein, 7 aa, synthetic construct
  REGION 1..7: note = Synthetic peptide
EIVYKPV

SEQ ID NO: 45 - protein, 7 aa, synthetic construct
  REGION 1..7: note = Synthetic peptide
IVYKSPV

SEQ ID NO: 46 - protein, 4 aa, synthetic construct
  REGION 1..4: note = Synthetic peptide
IVYK

SEQ ID NO: 47 - protein, 7 aa, synthetic construct
  REGION 1..7: note = Synthetic peptide
CNIKHVP

SEQ ID NO: 48 - protein, 6 aa, synthetic construct
  REGION 1..6: note = Synthetic peptide
NIKHVP

SEQ ID NO: 49 - protein, 6 aa, synthetic construct
  REGION 1..6: note = Synthetic peptide
HVPGGG

SEQ ID NO: 50 - protein, 5 aa, synthetic construct
  REGION 1..5: note = Synthetic peptide
HVPGG

SEQ ID NO: 51 - protein, 6 aa, synthetic construct
  REGION 1..6: note = Synthetic peptide
HKPGGG

SEQ ID NO: 52 - protein, 5 aa, synthetic construct
  REGION 1..5: note = Synthetic peptide
HKPGG

SEQ ID NO: 53 - protein, 7 aa, synthetic construct
  REGION 1..7: note = Synthetic peptide
KHVPGGG

SEQ ID NO: 54 - protein, 6 aa, synthetic construct
  REGION 1..6: note = Synthetic peptide
KHVPGG

SEQ ID NO: 55 - protein, 6 aa, synthetic construct
  REGION 1..6: note = Synthetic peptide
HQPGGG

SEQ ID NO: 56 - protein, 5 aa, synthetic construct
  REGION 1..5: note = Synthetic peptide
HQPGG

SEQ ID NO: 57 - protein, 16 aa, synthetic construct
  REGION 1..16: note = Synthetic peptide
DAEFRHDRRQIVYKPV

SEQ ID NO: 58 - protein, 16 aa, synthetic construct
  REGION 1..16: note = Synthetic peptide
DAEFRHDRREIVYKSV

SEQ ID NO: 59 - protein, 19 aa, synthetic construct
  REGION 1..19: note = Synthetic peptide
DAEFRHDRRQIVYKPVGGC

SEQ ID NO: 60 - protein, 17 aa, synthetic construct
  REGION 1..17: note = Synthetic peptide
DAEFRHDRRQIVYKPVC

SEQ ID NO: 61 - protein, 19 aa, synthetic construct
  REGION 1..19: note = Synthetic peptide
DAEFRHDRRQIVYKPVAAC

SEQ ID NO: 62 - protein, 19 aa, synthetic construct
  REGION 1..19: note = Synthetic peptide
DAEFRHDRRQIVYKPVKKC

SEQ ID NO: 63 - protein, 19 aa, synthetic construct
  REGION 1..19: note = Synthetic peptide
DAEFRHDRREIVYKSPGGC

SEQ ID NO: 64 - protein, 19 aa, synthetic construct
  REGION 1..19: note = Synthetic peptide
DAEFRHDRREIVYKSPAAC

SEQ ID NO: 65 - protein, 17 aa, synthetic construct
  REGION 1..17: note = Synthetic peptide
DAEFRHDRREIVYKSPC

SEQ ID NO: 66 - protein, 19 aa, synthetic construct
  REGION 1..19: note = Synthetic peptide
DAEFRHDRREIVYKSPKKC

SEQ ID NO: 67 - protein, 28 aa, synthetic construct
  REGION 1..28: note = Synthetic peptide
DAEFRHDSGYEVHHQKLFFAEDVGSNKG

SEQ ID NO: 68 - protein, 15 aa, synthetic construct
  REGION 1..15: note = Synthetic peptide
GGGSVQIVYKPVDLS

SEQ ID NO: 69 - protein, 4 aa, synthetic construct
  REGION 1..4: note = Synthetic peptide
RVRR

SEQ ID NO: 70 - protein, 19 aa, synthetic construct
  REGION 1..19: note = Synthetic peptide
  VAR_SEQ 17: note = MISC_FEATURE - Xaa can be GG or AA or KK or SS
  VAR_SEQ 18: note = MISC_FEATURE - Xaacan be GG or AA or KK or SS
DAEFRHDRRQIVYKPVXXC

SEQ ID NO: 71 - protein, 8 aa, synthetic construct
  REGION 1..8: note = Synthetic peptide
DAEFRHDC

SEQ ID NO: 72 - protein, 10 aa, synthetic construct
  REGION 1..10: note = Synthetic peptide
QIVYKPVGGC

SEQ ID NO: 73 - protein, 13 aa, synthetic construct
  REGION 1..13: note = Synthetic peptide
GGSQIVYKPVDLS

SEQ ID NO: 74 - protein, 19 aa, synthetic construct
  REGION 1..19: note = Synthetic peptide
DAEFRHDRREIVYKSVGGC

SEQ ID NO: 75 - protein, 19 aa, synthetic construct
  REGION 1..19: note = Synthetic peptide
DAEFRHDRREIVYKSVAAC

SEQ ID NO: 76 - protein, 17 aa, synthetic construct
  REGION 1..17: note = Synthetic peptide
DAEFRHDRREIVYKSVC

SEQ ID NO: 77 - protein, 19 aa, synthetic construct
  REGION 1..19: note = Synthetic peptide
DAEFRHDRREIVYKSVKKC

SEQ ID NO: 78 - protein, 18 aa, synthetic construct
  REGION 1..18: note = Synthetic peptide
DAEFRHDRRNIKHVPGGC

SEQ ID NO: 79 - protein, 19 aa, synthetic construct
  REGION 1..19: note = Synthetic peptide
  VAR_SEQ 17: note = MISC_FEATURE - Xaa can be GG or AA or KK or SS
  VAR_SEQ 18: note = MISC_FEATURE - Xaa can be GG or AA or KK or SS
DAEFRHDRREIVYKSVXXC

SEQ ID NO: 80 - protein, 4 aa, synthetic construct
  REGION 1..4: note = Synthetic peptide
GAGA

SEQ ID NO: 81 - protein, 4 aa, synthetic construct
  REGION 1..4: note = Synthetic peptide
AGAG

SEQ ID NO: 82 - protein, 4 aa, synthetic construct
  REGION 1..4: note = Synthetic peptide
KGKG

SEQ ID NO: 83 - protein, 6 aa, synthetic construct
  REGION 1..6: note = Synthetic peptide
QIVYKS

SEQ ID NO: 84 - protein, 6 aa, synthetic construct
  REGION 1..6: note = Synthetic peptide
EIVYKS

SEQ ID NO: 85 - protein, 6 aa, synthetic construct
  REGION 1..6: note = Synthetic peptide
EIVYKP

SEQ ID NO: 86 - protein, 8 aa, synthetic construct
  REGION 1..8: note = Synthetic peptide
CNIKHVPG

SEQ ID NO: 87-145 - skipped (59 entries)

SEQ ID NO: 146 - protein, 6 aa, synthetic construct
  REGION 1..6: note = Synthetic peptide
VQIINK

SEQ ID NO: 147 - protein, 7 aa, synthetic construct
  REGION 1..7: note = Synthetic peptide
VQIINKK

SEQ ID NO: 148 - protein, 8 aa, synthetic construct
  REGION 1..8: note = Synthetic peptide
VQIINKKL

SEQ ID NO: 149 - protein, 5 aa, synthetic construct
  REGION 1..5: note = Synthetic peptide
QIINK

SEQ ID NO: 150 - protein, 6 aa, synthetic construct
  REGION 1..6: note = Synthetic peptide
QIINKK

SEQ ID NO: 151 - protein, 7 aa, synthetic construct
  REGION 1..7: note = Synthetic peptide
QIINKKL

SEQ ID NO: 152 - protein, 9 aa, synthetic construct
  REGION 1..9: note = Synthetic peptide
EAAGHVTQC

SEQ ID NO: 153 - protein, 10 aa, synthetic construct
  REGION 1..10: note = Synthetic peptide
EAAGHVTQAR

SEQ ID NO: 154 - protein, 9 aa, synthetic construct
  REGION 1..9: note = Synthetic peptide
AAGHVTQAC

SEQ ID NO: 155 - protein, 9 aa, synthetic construct
  REGION 1..9: note = Synthetic peptide
AGHVTQARC

SEQ ID NO: 156 - protein, 8 aa, synthetic construct
  REGION 1..8: note = Synthetic peptide
AGHVTQAR

SEQ ID NO: 157 - protein, 7 aa, synthetic construct
  REGION 1..7: note = Synthetic peptide
GYTMHQD

SEQ ID NO: 158 - protein, 8 aa, synthetic construct
  REGION 1..8: note = Synthetic peptide
QGGYTMHC

SEQ ID NO: 159 - protein, 9 aa, synthetic construct
  REGION 1..9: note = Synthetic peptide
QGGYTMHQD

SEQ ID NO: 160 - protein, 8 aa, synthetic construct
  REGION 1..8: note = Synthetic peptide
GGYTMHQC

SEQ ID NO: 161 - protein, 10 aa, synthetic construct
  REGION 1..10: note = Synthetic peptide
VPGGGSVQIV

SEQ ID NO: 162 - protein, 9 aa, synthetic construct
  REGION 1..9: note = Synthetic peptide
PGGGSVQIV

SEQ ID NO: 163 - protein, 8 aa, synthetic construct
  REGION 1..8: note = Synthetic peptide
GGGSVQIV

SEQ ID NO: 164 - protein, 7 aa, synthetic construct
  REGION 1..7: note = Synthetic peptide
GGSVQIV

SEQ ID NO: 165 - protein, 6 aa, synthetic construct
  REGION 1..6: note = Synthetic peptide
GSVQIV

SEQ ID NO: 166 - protein, 5 aa, synthetic construct
  REGION 1..5: note = Synthetic peptide
SVQIV

SEQ ID NO: 167 - protein, 4 aa, synthetic construct
  REGION 1..4: note = Synthetic peptide
VQIV

SEQ ID NO: 168 - skipped

SEQ ID NO: 169 - protein, 10 aa, synthetic construct
  REGION 1..10: note = Synthetic peptide
PGGGSVQIVY

SEQ ID NO: 170 - protein, 9 aa, synthetic construct
  REGION 1..9: note = Synthetic peptide
GGGSVQIVY

SEQ ID NO: 171 - protein, 8 aa, synthetic construct
  REGION 1..8: note = Synthetic peptide
GGSVQIVY

SEQ ID NO: 172 - protein, 7 aa, synthetic construct
  REGION 1..7: note = Synthetic peptide
GSVQIVY

SEQ ID NO: 173 - protein, 6 aa, synthetic construct
  REGION 1..6: note = Synthetic peptide
SVQIVY

SEQ ID NO: 174 - protein, 5 aa, synthetic construct
  REGION 1..5: note = Synthetic peptide
VQIVY

SEQ ID NO: 175 - protein, 4 aa, synthetic construct
  REGION 1..4: note = Synthetic peptide
QIVY

SEQ ID NO: 176 - skipped

SEQ ID NO: 177 - protein, 10 aa, synthetic construct
  REGION 1..10: note = Synthetic peptide
GGGSVQIVYK

SEQ ID NO: 178 - protein, 9 aa, synthetic construct
  REGION 1..9: note = Synthetic peptide
GGSVQIVYK

SEQ ID NO: 179 - protein, 8 aa, synthetic construct
  REGION 1..8: note = Synthetic peptide
GSVQIVYK

SEQ ID NO: 180 - protein, 7 aa, synthetic construct
  REGION 1..7: note = Synthetic peptide
SVQIVYK

SEQ ID NO: 181 - protein, 6 aa, synthetic construct
  REGION 1..6: note = Synthetic peptide
VQIVYK

SEQ ID NO: 182 - protein, 5 aa, synthetic construct
  REGION 1..5: note = Synthetic peptide
QIVYK

SEQ ID NO: 183 - skipped

SEQ ID NO: 184 - protein, 10 aa, synthetic construct
  REGION 1..10: note = Synthetic peptide
GGSVQIVYKP

SEQ ID NO: 185 - protein, 9 aa, synthetic construct
  REGION 1..9: note = Synthetic peptide
GSVQIVYKP

SEQ ID NO: 186 - protein, 8 aa, synthetic construct
  REGION 1..8: note = Synthetic peptide
SVQIVYKP

SEQ ID NO: 187 - protein, 7 aa, synthetic construct
  REGION 1..7: note = Synthetic peptide
VQIVYKP

SEQ ID NO: 188 - protein, 5 aa, synthetic construct
  REGION 1..5: note = Synthetic peptide
IVYKP

SEQ ID NO: 189 - protein, 4 aa, synthetic construct
  REGION 1..4: note = Synthetic peptide
VYKP

SEQ ID NO: 190 - skipped

SEQ ID NO: 191 - protein, 10 aa, synthetic construct
  REGION 1..10: note = Synthetic peptide
GSVQIVYKPV

SEQ ID NO: 192 - protein, 9 aa, synthetic construct
  REGION 1..9: note = Synthetic peptide
SVQIVYKPV

SEQ ID NO: 193 - protein, 8 aa, synthetic construct
  REGION 1..8: note = Synthetic peptide
VQIVYKPV

SEQ ID NO: 194 - protein, 6 aa, synthetic construct
  REGION 1..6: note = Synthetic peptide
IVYKPV

SEQ ID NO: 195 - protein, 5 aa, synthetic construct
  REGION 1..5: note = Synthetic peptide
VYKPV

SEQ ID NO: 196 - protein, 4 aa, synthetic construct
  REGION 1..4: note = Synthetic peptide
YKPV

SEQ ID NO: 197 - skipped

SEQ ID NO: 198 - protein, 10 aa, synthetic construct
  REGION 1..10: note = Synthetic peptide
SVQIVYKPVD

SEQ ID NO: 199 - protein, 9 aa, synthetic construct
  REGION 1..9: note = Synthetic peptide
VQIVYKPVD

SEQ ID NO: 200 - protein, 8 aa, synthetic construct
  REGION 1..8: note = Synthetic peptide
QIVYKPVD

SEQ ID NO: 201 - protein, 7 aa, synthetic construct
  REGION 1..7: note = Synthetic peptide
IVYKPVD

SEQ ID NO: 202 - skipped

SEQ ID NO: 203 - protein, 6 aa, synthetic construct
  REGION 1..6: note = Synthetic peptide
VYKPVD

SEQ ID NO: 204 - protein, 5 aa, synthetic construct
  REGION 1..5: note = Synthetic peptide
YKPVD

SEQ ID NO: 205 - protein, 4 aa, synthetic construct
  REGION 1..4: note = Synthetic peptide
KPVD

SEQ ID NO: 206 - skipped

SEQ ID NO: 207 - protein, 10 aa, synthetic construct
  REGION 1..10: note = Synthetic peptide
VQIVYKPVDL

SEQ ID NO: 208 - protein, 9 aa, synthetic construct
  REGION 1..9: note = Synthetic peptide
QIVYKPVDL

SEQ ID NO: 209 - protein, 8 aa, synthetic construct
  REGION 1..8: note = Synthetic peptide
IVYKPVDL

SEQ ID NO: 210 - protein, 7 aa, synthetic construct
  REGION 1..7: note = Synthetic peptide
VYKPVDL

SEQ ID NO: 211 - protein, 6 aa, synthetic construct
  REGION 1..6: note = Synthetic peptide
YKPVDL

SEQ ID NO: 212 - protein, 5 aa, synthetic construct
  REGION 1..5: note = Synthetic peptide
KPVDL

SEQ ID NO: 213 - protein, 4 aa, synthetic construct
  REGION 1..4: note = Synthetic peptide
PVDL

SEQ ID NO: 214 - skipped

SEQ ID NO: 215 - protein, 10 aa, synthetic construct
  REGION 1..10: note = Synthetic peptide
QIVYKPVDLS

SEQ ID NO: 216 - protein, 9 aa, synthetic construct
  REGION 1..9: note = Synthetic peptide
IVYKPVDLS

SEQ ID NO: 217 - protein, 8 aa, synthetic construct
  REGION 1..8: note = Synthetic peptide
VYKPVDLS

SEQ ID NO: 218 - protein, 7 aa, synthetic construct
  REGION 1..7: note = Synthetic peptide
YKPVDLS

SEQ ID NO: 219 - protein, 6 aa, synthetic construct
  REGION 1..6: note = Synthetic peptide
KPVDLS

SEQ ID NO: 220 - protein, 5 aa, synthetic construct
  REGION 1..5: note = Synthetic peptide
PVDLS

SEQ ID NO: 221 - protein, 4 aa, synthetic construct
  REGION 1..4: note = Synthetic peptide
VDLS

SEQ ID NO: 222 - skipped

SEQ ID NO: 223 - protein, 10 aa, synthetic construct
  REGION 1..10: note = Synthetic peptide
IVYKPVDLSK

SEQ ID NO: 224 - protein, 9 aa, synthetic construct
  REGION 1..9: note = Synthetic peptide
VYKPVDLSK

SEQ ID NO: 225 - protein, 8 aa, synthetic construct
  REGION 1..8: note = Synthetic peptide
YKPVDLSK

SEQ ID NO: 226 - protein, 7 aa, synthetic construct
  REGION 1..7: note = Synthetic peptide
KPVDLSK

SEQ ID NO: 227 - protein, 6 aa, synthetic construct
  REGION 1..6: note = Synthetic peptide
PVDLSK

SEQ ID NO: 228 - protein, 5 aa, synthetic construct
  REGION 1..5: note = Synthetic peptide
VDLSK

SEQ ID NO: 229 - protein, 4 aa, synthetic construct
  REGION 1..4: note = Synthetic peptide
DLSK

SEQ ID NO: 230 - skipped

SEQ ID NO: 231 - protein, 10 aa, synthetic construct
  REGION 1..10: note = Synthetic peptide
VYKPVDLSKV

SEQ ID NO: 232 - protein, 9 aa, synthetic construct
  REGION 1..9: note = Synthetic peptide
YKPVDLSKV

SEQ ID NO: 233 - protein, 8 aa, synthetic construct
  REGION 1..8: note = Synthetic peptide
KPVDLSKV

SEQ ID NO: 234 - protein, 7 aa, synthetic construct
  REGION 1..7: note = Synthetic peptide
PVDLSKV

SEQ ID NO: 235 - protein, 6 aa, synthetic construct
  REGION 1..6: note = Synthetic peptide
VDLSKV

SEQ ID NO: 236 - protein, 5 aa, synthetic construct
  REGION 1..5: note = Synthetic peptide
DLSKV

SEQ ID NO: 237 - protein, 4 aa, synthetic construct
  REGION 1..4: note = Synthetic peptide
LSKV

SEQ ID NO: 238 - skipped

SEQ ID NO: 239 - protein, 10 aa, synthetic construct
  REGION 1..10: note = Synthetic peptide
YKPVDLSKVT

SEQ ID NO: 240 - protein, 9 aa, synthetic construct
  REGION 1..9: note = Synthetic peptide
KPVDLSKVT

SEQ ID NO: 241 - protein, 8 aa, synthetic construct
  REGION 1..8: note = Synthetic peptide
PVDLSKVT

SEQ ID NO: 242 - protein, 7 aa, synthetic construct
  REGION 1..7: note = Synthetic peptide
VDLSKVT

SEQ ID NO: 243 - protein, 10 aa, synthetic construct
  REGION 1..10: note = Synthetic peptide
AKTDHGAEIV

SEQ ID NO: 244 - protein, 9 aa, synthetic construct
  REGION 1..9: note = Synthetic peptide
KTDHGAEIV

SEQ ID NO: 245 - protein, 8 aa, synthetic construct
  REGION 1..8: note = Synthetic peptide
TDHGAEIV

SEQ ID NO: 246 - protein, 7 aa, synthetic construct
  REGION 1..7: note = Synthetic peptide
DHGAEIV

SEQ ID NO: 247 - protein, 6 aa, synthetic construct
  REGION 1..6: note = Synthetic peptide
HGAEIV

SEQ ID NO: 248 - protein, 5 aa, synthetic construct
  REGION 1..5: note = Synthetic peptide
GAEIV

SEQ ID NO: 249 - protein, 4 aa, synthetic construct
  REGION 1..4: note = Synthetic peptide
AEIV

SEQ ID NO: 250 - skipped

SEQ ID NO: 251 - protein, 10 aa, synthetic construct
  REGION 1..10: note = Synthetic peptide
KTDHGAEIVY

SEQ ID NO: 252 - protein, 9 aa, synthetic construct
  REGION 1..9: note = Synthetic peptide
TDHGAEIVY

SEQ ID NO: 253 - protein, 8 aa, synthetic construct
  REGION 1..8: note = Synthetic peptide
DHGAEIVY

SEQ ID NO: 254 - protein, 7 aa, synthetic construct
  REGION 1..7: note = Synthetic peptide
HGAEIVY

SEQ ID NO: 255 - protein, 6 aa, synthetic construct
  REGION 1..6: note = Synthetic peptide
GAEIVY

SEQ ID NO: 256 - protein, 5 aa, synthetic construct
  REGION 1..5: note = Synthetic peptide
AEIVY

SEQ ID NO: 257 - protein, 4 aa, synthetic construct
  REGION 1..4: note = Synthetic peptide
EIVY

SEQ ID NO: 258 - skipped

SEQ ID NO: 259 - protein, 10 aa, synthetic construct
  REGION 1..10: note = Synthetic peptide
TDHGAEIVYK

SEQ ID NO: 260 - protein, 9 aa, synthetic construct
  REGION 1..9: note = Synthetic peptide
DHGAEIVYK

SEQ ID NO: 261 - protein, 8 aa, synthetic construct
  REGION 1..8: note = Synthetic peptide
HGAEIVYK

SEQ ID NO: 262 - protein, 7 aa, synthetic construct
  REGION 1..7: note = Synthetic peptide
GAEIVYK

SEQ ID NO: 263 - protein, 6 aa, synthetic construct
  REGION 1..6: note = Synthetic peptide
AEIVYK

SEQ ID NO: 264 - protein, 5 aa, synthetic construct
  REGION 1..5: note = Synthetic peptide
EIVYK

SEQ ID NO: 265 - protein, 4 aa, synthetic construct
  REGION 1..4: note = Synthetic peptide
IVYK

SEQ ID NO: 266 - protein, 10 aa, synthetic construct
  REGION 1..10: note = Synthetic peptide
DHGAEIVYKS

SEQ ID NO: 267 - protein, 9 aa, synthetic construct
  REGION 1..9: note = Synthetic peptide
HGAEIVYKS

SEQ ID NO: 268 - protein, 8 aa, synthetic construct
  REGION 1..8: note = Synthetic peptide
GAEIVYKS

SEQ ID NO: 269 - protein, 7 aa, synthetic construct
  REGION 1..7: note = Synthetic peptide
AEIVYKS

SEQ ID NO: 270 - protein, 6 aa, synthetic construct
  REGION 1..6: note = Synthetic peptide
EIVYKS

SEQ ID NO: 271 - protein, 5 aa, synthetic construct
  REGION 1..5: note = Synthetic peptide
IVYKS

SEQ ID NO: 272 - protein, 4 aa, synthetic construct
  REGION 1..4: note = Synthetic peptide
VYKS

SEQ ID NO: 273 - skipped

SEQ ID NO: 274 - protein, 10 aa, synthetic construct
  REGION 1..10: note = Synthetic peptide
HGAEIVYKSP

SEQ ID NO: 275 - protein, 9 aa, synthetic construct
  REGION 1..9: note = Synthetic peptide
GAEIVYKSP

SEQ ID NO: 276 - protein, 8 aa, synthetic construct
  REGION 1..8: note = Synthetic peptide
AEIVYKSP

SEQ ID NO: 277 - protein, 7 aa, synthetic construct
  REGION 1..7: note = Synthetic peptide
EIVYKSP

SEQ ID NO: 278 - protein, 6 aa, synthetic construct
  REGION 1..6: note = Synthetic peptide
IVYKSP

SEQ ID NO: 279 - protein, 5 aa, synthetic construct
  REGION 1..5: note = Synthetic peptide
VYKSP

SEQ ID NO: 280 - protein, 4 aa, synthetic construct
  REGION 1..4: note = Synthetic peptide
YKSP

SEQ ID NO: 281 - skipped

SEQ ID NO: 282 - protein, 10 aa, synthetic construct
  REGION 1..10: note = Synthetic peptide
GAEIVYKSPV

SEQ ID NO: 283 - protein, 9 aa, synthetic construct
  REGION 1..9: note = Synthetic peptide
AEIVYKSPV

SEQ ID NO: 284 - protein, 8 aa, synthetic construct
  REGION 1..8: note = Synthetic peptide
EIVYKSPV

SEQ ID NO: 285 - protein, 7 aa, synthetic construct
  REGION 1..7: note = Synthetic peptide
IVYKSPV

SEQ ID NO: 286 - protein, 6 aa, synthetic construct
  REGION 1..6: note = Synthetic peptide
VYKSPV

SEQ ID NO: 287 - protein, 5 aa, synthetic construct
  REGION 1..5: note = Synthetic peptide
YKSPV

SEQ ID NO: 288 - protein, 4 aa, synthetic construct
  REGION 1..4: note = Synthetic peptide
KSPV

SEQ ID NO: 289 - skipped

SEQ ID NO: 290 - protein, 10 aa, synthetic construct
  REGION 1..10: note = Synthetic peptide
AEIVYKSPVV

SEQ ID NO: 291 - protein, 9 aa, synthetic construct
  REGION 1..9: note = Synthetic peptide
EIVYKSPVV

SEQ ID NO: 292 - protein, 8 aa, synthetic construct
  REGION 1..8: note = Synthetic peptide
IVYKSPVV

SEQ ID NO: 293 - protein, 7 aa, synthetic construct
  REGION 1..7: note = Synthetic peptide
VYKSPVV

SEQ ID NO: 294 - protein, 6 aa, synthetic construct
  REGION 1..6: note = Synthetic peptide
YKSPVV

SEQ ID NO: 295 - protein, 5 aa, synthetic construct
  REGION 1..5: note = Synthetic peptide
KSPVV

SEQ ID NO: 296 - protein, 4 aa, synthetic construct
  REGION 1..4: note = Synthetic peptide
SPVV

SEQ ID NO: 297 - skipped

SEQ ID NO: 298 - protein, 10 aa, synthetic construct
  REGION 1..10: note = Synthetic peptide
EIVYKSPVVS

SEQ ID NO: 299 - protein, 9 aa, synthetic construct
  REGION 1..9: note = Synthetic peptide
IVYKSPVVS

SEQ ID NO: 300 - protein, 8 aa, synthetic construct
  REGION 1..8: note = Synthetic peptide
VYKSPVVS

SEQ ID NO: 301 - protein, 7 aa, synthetic construct
  REGION 1..7: note = Synthetic peptide
YKSPVVS

SEQ ID NO: 302 - protein, 6 aa, synthetic construct
  REGION 1..6: note = Synthetic peptide
KSPVVS

SEQ ID NO: 303 - protein, 5 aa, synthetic construct
  REGION 1..5: note = Synthetic peptide
SPVVS

SEQ ID NO: 304 - protein, 4 aa, synthetic construct
  REGION 1..4: note = Synthetic peptide
PVVS

SEQ ID NO: 305 - skipped

SEQ ID NO: 306 - protein, 10 aa, synthetic construct
  REGION 1..10: note = Synthetic peptide
IVYKSPVVSG

SEQ ID NO: 307 - protein, 9 aa, synthetic construct
  REGION 1..9: note = Synthetic peptide
VYKSPVVSG

SEQ ID NO: 308 - protein, 8 aa, synthetic construct
  REGION 1..8: note = Synthetic peptide
YKSPVVSG

SEQ ID NO: 309 - protein, 7 aa, synthetic construct
  REGION 1..7: note = Synthetic peptide
KSPVVSG

SEQ ID NO: 310 - protein, 6 aa, synthetic construct
  REGION 1..6: note = Synthetic peptide
SPVVSG

SEQ ID NO: 311 - protein, 5 aa, synthetic construct
  REGION 1..5: note = Synthetic peptide
PVVSG

SEQ ID NO: 312 - protein, 4 aa, synthetic construct
  REGION 1..4: note = Synthetic peptide
VVSG

SEQ ID NO: 313 - skipped

SEQ ID NO: 314 - protein, 10 aa, synthetic construct
  REGION 1..10: note = Synthetic peptide
VYKSPVVSGD

SEQ ID NO: 315 - protein, 9 aa, synthetic construct
  REGION 1..9: note = Synthetic peptide
YKSPVVSGD

SEQ ID NO: 316 - protein, 8 aa, synthetic construct
  REGION 1..8: note = Synthetic peptide
KSPVVSGD

SEQ ID NO: 317 - protein, 7 aa, synthetic construct
  REGION 1..7: note = Synthetic peptide
SPVVSGD

SEQ ID NO: 318 - protein, 6 aa, synthetic construct
  REGION 1..6: note = Synthetic peptide
PVVSGD

SEQ ID NO: 319 - protein, 5 aa, synthetic construct
  REGION 1..5: note = Synthetic peptide
VVSGD

SEQ ID NO: 320 - protein, 4 aa, synthetic construct
  REGION 1..4: note = Synthetic peptide
VSGD

SEQ ID NO: 321 - skipped

SEQ ID NO: 322 - protein, 10 aa, synthetic construct
  REGION 1..10: note = Synthetic peptide
YKSPVVSGDT

SEQ ID NO: 323 - protein, 9 aa, synthetic construct
  REGION 1..9: note = Synthetic peptide
KSPVVSGDT

SEQ ID NO: 324 - protein, 8 aa, synthetic construct
  REGION 1..8: note = Synthetic peptide
SPVVSGDT

SEQ ID NO: 325 - protein, 7 aa, synthetic construct
  REGION 1..7: note = Synthetic peptide
PVVSGDT

SEQ ID NO: 326 - protein, 6 aa, synthetic construct
  REGION 1..6: note = Synthetic peptide
VVSGDT

SEQ ID NO: 327 - protein, 5 aa, synthetic construct
  REGION 1..5: note = Synthetic peptide
VSGDT

SEQ ID NO: 328 - protein, 4 aa, synthetic construct
  REGION 1..4: note = Synthetic peptide
SGDT

SEQ ID NO: 329 - skipped

SEQ ID NO: 330 - protein, 10 aa, synthetic construct
  REGION 1..10: note = Synthetic peptide
KSPVVSGDTS

SEQ ID NO: 331 - protein, 9 aa, synthetic construct
  REGION 1..9: note = Synthetic peptide
SPVVSGDTS

SEQ ID NO: 332 - protein, 8 aa, synthetic construct
  REGION 1..8: note = Synthetic peptide
PVVSGDTS

SEQ ID NO: 333 - protein, 7 aa, synthetic construct
  REGION 1..7: note = Synthetic peptide
VVSGDTS

SEQ ID NO: 334 - protein, 6 aa, synthetic construct
  REGION 1..6: note = Synthetic peptide
VSGDTS

SEQ ID NO: 335 - protein, 5 aa, synthetic construct
  REGION 1..5: note = Synthetic peptide
SGDTS

SEQ ID NO: 336 - protein, 4 aa, synthetic construct
  REGION 1..4: note = Synthetic peptide
GDTS

SEQ ID NO: 337 - skipped

SEQ ID NO: 338 - protein, 10 aa, synthetic construct
  REGION 1..10: note = Synthetic peptide
SPVVSGDTSP

SEQ ID NO: 339 - protein, 9 aa, synthetic construct
  REGION 1..9: note = Synthetic peptide
PVVSGDTSP

SEQ ID NO: 340 - protein, 8 aa, synthetic construct
  REGION 1..8: note = Synthetic peptide
VVSGDTSP

SEQ ID NO: 341 - protein, 7 aa, synthetic construct
  REGION 1..7: note = Synthetic peptide
VSGDTSP

SEQ ID NO: 342 - protein, 6 aa, synthetic construct
  REGION 1..6: note = Synthetic peptide
SGDTSP

SEQ ID NO: 343 - protein, 5 aa, synthetic construct
  REGION 1..5: note = Synthetic peptide
GDTSP

SEQ ID NO: 344 - protein, 4 aa, synthetic construct
  REGION 1..4: note = Synthetic peptide
DTSP

SEQ ID NO: 345 - skipped

SEQ ID NO: 346 - protein, 10 aa, synthetic construct
  REGION 1..10: note = Synthetic peptide
PVVSGDTSPR

SEQ ID NO: 347 - protein, 9 aa, synthetic construct
  REGION 1..9: note = Synthetic peptide
VVSGDTSPR

SEQ ID NO: 348 - protein, 8 aa, synthetic construct
  REGION 1..8: note = Synthetic peptide
VSGDTSPR

SEQ ID NO: 349 - protein, 7 aa, synthetic construct
  REGION 1..7: note = Synthetic peptide
SGDTSPR

SEQ ID NO: 350 - protein, 6 aa, synthetic construct
  REGION 1..6: note = Synthetic peptide
GDTSPR

SEQ ID NO: 351 - protein, 5 aa, synthetic construct
  REGION 1..5: note = Synthetic peptide
DTSPR

SEQ ID NO: 352 - protein, 4 aa, synthetic construct
  REGION 1..4: note = Synthetic peptide
TSPR

SEQ ID NO: 353 - skipped

SEQ ID NO: 354 - protein, 10 aa, synthetic construct
  REGION 1..10: note = Synthetic peptide
HQPGGGKVQI

SEQ ID NO: 355 - protein, 9 aa, synthetic construct
  REGION 1..9: note = Synthetic peptide
QPGGGKVQI

SEQ ID NO: 356 - protein, 8 aa, synthetic construct
  REGION 1..8: note = Synthetic peptide
PGGGKVQI

SEQ ID NO: 357 - protein, 7 aa, synthetic construct
  REGION 1..7: note = Synthetic peptide
GGGKVQI

SEQ ID NO: 358 - protein, 6 aa, synthetic construct
  REGION 1..6: note = Synthetic peptide
GGKVQI

SEQ ID NO: 359 - protein, 5 aa, synthetic construct
  REGION 1..5: note = Synthetic peptide
GKVQI

SEQ ID NO: 360 - protein, 4 aa, synthetic construct
  REGION 1..4: note = Synthetic peptide
KVQI

SEQ ID NO: 361 - skipped

SEQ ID NO: 362 - protein, 10 aa, synthetic construct
  REGION 1..10: note = Synthetic peptide
QPGGGKVQII

SEQ ID NO: 363 - protein, 9 aa, synthetic construct
  REGION 1..9: note = Synthetic peptide
PGGGKVQII

SEQ ID NO: 364 - protein, 6 aa, synthetic construct
  REGION 1..6: note = Synthetic peptide
SVDLSK

SEQ ID NO: 365 - protein, 8 aa, synthetic construct
  REGION 1..8: note = Synthetic peptide
GGGKVQII

SEQ ID NO: 366 - protein, 7 aa, synthetic construct
  REGION 1..7: note = Synthetic peptide
GGKVQII

SEQ ID NO: 367 - protein, 6 aa, synthetic construct
  REGION 1..6: note = Synthetic peptide
GKVQII

SEQ ID NO: 368 - protein, 5 aa, synthetic construct
  REGION 1..5: note = Synthetic peptide
KVQII

SEQ ID NO: 369 - protein, 4 aa, synthetic construct
  REGION 1..4: note = Synthetic peptide
VQII

SEQ ID NO: 370 - skipped

SEQ ID NO: 371 - protein, 10 aa, synthetic construct
  REGION 1..10: note = Synthetic peptide
PGGGKVQIIN

SEQ ID NO: 372 - protein, 9 aa, synthetic construct
  REGION 1..9: note = Synthetic peptide
GGGKVQIIN

SEQ ID NO: 373 - protein, 8 aa, synthetic construct
  REGION 1..8: note = Synthetic peptide
GGKVQIIN

SEQ ID NO: 374 - protein, 7 aa, synthetic construct
  REGION 1..7: note = Synthetic peptide
GKVQIIN

SEQ ID NO: 375 - protein, 6 aa, synthetic construct
  REGION 1..6: note = Synthetic peptide
KVQIIN

SEQ ID NO: 376 - protein, 5 aa, synthetic construct
  REGION 1..5: note = Synthetic peptide
VQIIN

SEQ ID NO: 377 - protein, 4 aa, synthetic construct
  REGION 1..4: note = Synthetic peptide
QIIN

SEQ ID NO: 378 - skipped

SEQ ID NO: 379 - protein, 10 aa, synthetic construct
  REGION 1..10: note = Synthetic peptide
GGGKVQIINK

SEQ ID NO: 380 - protein, 9 aa, synthetic construct
  REGION 1..9: note = Synthetic peptide
GGKVQIINK

SEQ ID NO: 381 - protein, 8 aa, synthetic construct
  REGION 1..8: note = Synthetic peptide
GKVQIINK

SEQ ID NO: 382 - protein, 7 aa, synthetic construct
  REGION 1..7: note = Synthetic peptide
KVQIINK

SEQ ID NO: 383 - protein, 4 aa, synthetic construct
  REGION 1..4: note = Synthetic peptide
IINK

SEQ ID NO: 384 - skipped

SEQ ID NO: 385 - protein, 10 aa, synthetic construct
  REGION 1..10: note = Synthetic peptide
GGKVQIINKK

SEQ ID NO: 386 - protein, 9 aa, synthetic construct
  REGION 1..9: note = Synthetic peptide
GKVQIINKK

SEQ ID NO: 387 - protein, 8 aa, synthetic construct
  REGION 1..8: note = Synthetic peptide
KVQIINKK

SEQ ID NO: 388 - protein, 5 aa, synthetic construct
  REGION 1..5: note = Synthetic peptide
IINKK

SEQ ID NO: 389 - protein, 4 aa, synthetic construct
  REGION 1..4: note = Synthetic peptide
INKK

SEQ ID NO: 390 - skipped

SEQ ID NO: 391 - protein, 10 aa, synthetic construct
  REGION 1..10: note = Synthetic peptide
GKVQIINKKL

SEQ ID NO: 392 - protein, 9 aa, synthetic construct
  REGION 1..9: note = Synthetic peptide
KVQIINKKL

SEQ ID NO: 393 - protein, 6 aa, synthetic construct
  REGION 1..6: note = Synthetic peptide
IINKKL

SEQ ID NO: 394 - protein, 5 aa, synthetic construct
  REGION 1..5: note = Synthetic peptide
INKKL

SEQ ID NO: 395 - protein, 4 aa, synthetic construct
  REGION 1..4: note = Synthetic peptide
NKKL

SEQ ID NO: 396 - skipped

SEQ ID NO: 397 - protein, 10 aa, synthetic construct
  REGION 1..10: note = Synthetic peptide
KVQIINKKLD

SEQ ID NO: 398 - protein, 9 aa, synthetic construct
  REGION 1..9: note = Synthetic peptide
VQIINKKLD

SEQ ID NO: 399 - protein, 8 aa, synthetic construct
  REGION 1..8: note = Synthetic peptide
QIINKKLD

SEQ ID NO: 400 - protein, 7 aa, synthetic construct
  REGION 1..7: note = Synthetic peptide
IINKKLD

SEQ ID NO: 401 - protein, 6 aa, synthetic construct
  REGION 1..6: note = Synthetic peptide
INKKLD

SEQ ID NO: 402 - protein, 5 aa, synthetic construct
  REGION 1..5: note = Synthetic peptide
NKKLD

SEQ ID NO: 403 - protein, 4 aa, synthetic construct
  REGION 1..4: note = Synthetic peptide
KKLD

SEQ ID NO: 404 - skipped

SEQ ID NO: 405 - protein, 10 aa, synthetic construct
  REGION 1..10: note = Synthetic peptide
VQIINKKLDL

SEQ ID NO: 406 - protein, 9 aa, synthetic construct
  REGION 1..9: note = Synthetic peptide
QIINKKLDL

SEQ ID NO: 407 - protein, 8 aa, synthetic construct
  REGION 1..8: note = Synthetic peptide
IINKKLDL

SEQ ID NO: 408 - protein, 7 aa, synthetic construct
  REGION 1..7: note = Synthetic peptide
INKKLDL

SEQ ID NO: 409 - protein, 6 aa, synthetic construct
  REGION 1..6: note = Synthetic peptide
NKKLDL

SEQ ID NO: 410 - protein, 5 aa, synthetic construct
  REGION 1..5: note = Synthetic peptide
KKLDL

SEQ ID NO: 411 - protein, 4 aa, synthetic construct
  REGION 1..4: note = Synthetic peptide
KLDL

SEQ ID NO: 412 - skipped

SEQ ID NO: 413 - protein, 10 aa, synthetic construct
  REGION 1..10: note = Synthetic peptide
QIINKKLDLS

SEQ ID NO: 414 - protein, 9 aa, synthetic construct
  REGION 1..9: note = Synthetic peptide
IINKKLDLS

SEQ ID NO: 415 - protein, 8 aa, synthetic construct
  REGION 1..8: note = Synthetic peptide
INKKLDLS

SEQ ID NO: 416 - protein, 7 aa, synthetic construct
  REGION 1..7: note = Synthetic peptide
NKKLDLS

SEQ ID NO: 417 - protein, 6 aa, synthetic construct
  REGION 1..6: note = Synthetic peptide
KKLDLS

SEQ ID NO: 418 - protein, 5 aa, synthetic construct
  REGION 1..5: note = Synthetic peptide
KLDLS

SEQ ID NO: 419 - protein, 4 aa, synthetic construct
  REGION 1..4: note = Synthetic peptide
LDLS

SEQ ID NO: 420 - protein, 10 aa, synthetic construct
  REGION 1..10: note = Synthetic peptide
IINKKLDLSN

SEQ ID NO: 421 - protein, 9 aa, synthetic construct
  REGION 1..9: note = Synthetic peptide
INKKLDLSN

SEQ ID NO: 422 - protein, 8 aa, synthetic construct
  REGION 1..8: note = Synthetic peptide
NKKLDLSN

SEQ ID NO: 423 - protein, 7 aa, synthetic construct
  REGION 1..7: note = Synthetic peptide
KKLDLSN

SEQ ID NO: 424 - protein, 6 aa, synthetic construct
  REGION 1..6: note = Synthetic peptide
KLDLSN

SEQ ID NO: 425 - protein, 5 aa, synthetic construct
  REGION 1..5: note = Synthetic peptide
LDLSN

SEQ ID NO: 426 - protein, 4 aa, synthetic construct
  REGION 1..4: note = Synthetic peptide
DLSN

SEQ ID NO: 427 - skipped

SEQ ID NO: 428 - protein, 10 aa, synthetic construct
  REGION 1..10: note = Synthetic peptide
INKKLDLSNV

SEQ ID NO: 429 - protein, 9 aa, synthetic construct
  REGION 1..9: note = Synthetic peptide
NKKLDLSNV

SEQ ID NO: 430 - protein, 8 aa, synthetic construct
  REGION 1..8: note = Synthetic peptide
KKLDLSNV

SEQ ID NO: 431 - protein, 7 aa, synthetic construct
  REGION 1..7: note = Synthetic peptide
KLDLSNV

SEQ ID NO: 432 - protein, 6 aa, synthetic construct
  REGION 1..6: note = Synthetic peptide
LDLSNV

SEQ ID NO: 433 - protein, 5 aa, synthetic construct
  REGION 1..5: note = Synthetic peptide
DLSNV

SEQ ID NO: 434 - protein, 4 aa, synthetic construct
  REGION 1..4: note = Synthetic peptide
LSNV

SEQ ID NO: 435 - skipped

SEQ ID NO: 436 - protein, 10 aa, synthetic construct
  REGION 1..10: note = Synthetic peptide
NKKLDLSNVQ

SEQ ID NO: 437 - protein, 9 aa, synthetic construct
  REGION 1..9: note = Synthetic peptide
KKLDLSNVQ

SEQ ID NO: 438 - protein, 8 aa, synthetic construct
  REGION 1..8: note = Synthetic peptide
KLDLSNVQ

SEQ ID NO: 439 - protein, 7 aa, synthetic construct
  REGION 1..7: note = Synthetic peptide
LDLSNVQ

SEQ ID NO: 440 - protein, 6 aa, synthetic construct
  REGION 1..6: note = Synthetic peptide
DLSNVQ

SEQ ID NO: 441 - protein, 5 aa, synthetic construct
  REGION 1..5: note = Synthetic peptide
LSNVQ

SEQ ID NO: 442 - protein, 4 aa, synthetic construct
  REGION 1..4: note = Synthetic peptide
SNVQ

SEQ ID NO: 443 - skipped

SEQ ID NO: 444 - protein, 10 aa, synthetic construct
  REGION 1..10: note = Synthetic peptide
KKLDLSNVQS

SEQ ID NO: 445 - protein, 9 aa, synthetic construct
  REGION 1..9: note = Synthetic peptide
KLDLSNVQS

SEQ ID NO: 446 - protein, 8 aa, synthetic construct
  REGION 1..8: note = Synthetic peptide
LDLSNVQS

SEQ ID NO: 447 - protein, 7 aa, synthetic construct
  REGION 1..7: note = Synthetic peptide
DLSNVQS

SEQ ID NO: 448 - protein, 6 aa, synthetic construct
  REGION 1..6: note = Synthetic peptide
LSNVQS

SEQ ID NO: 449 - protein, 5 aa, synthetic construct
  REGION 1..5: note = Synthetic peptide
SNVQS

SEQ ID NO: 450 - protein, 4 aa, synthetic construct
  REGION 1..4: note = Synthetic peptide
NVQS

SEQ ID NO: 451 - skipped

SEQ ID NO: 452 - protein, 10 aa, synthetic construct
  REGION 1..10: note = Synthetic peptide
SKCGSKDNIK

SEQ ID NO: 453 - protein, 9 aa, synthetic construct
  REGION 1..9: note = Synthetic peptide
KCGSKDNIK

SEQ ID NO: 454 - protein, 8 aa, synthetic construct
  REGION 1..8: note = Synthetic peptide
CGSKDNIK

SEQ ID NO: 455 - protein, 7 aa, synthetic construct
  REGION 1..7: note = Synthetic peptide
GSKDNIK

SEQ ID NO: 456 - protein, 6 aa, synthetic construct
  REGION 1..6: note = Synthetic peptide
SKDNIK

SEQ ID NO: 457 - protein, 5 aa, synthetic construct
  REGION 1..5: note = Synthetic peptide
KDNIK

SEQ ID NO: 458 - protein, 4 aa, synthetic construct
  REGION 1..4: note = Synthetic peptide
DNIK

SEQ ID NO: 459 - skipped

SEQ ID NO: 460 - protein, 10 aa, synthetic construct
  REGION 1..10: note = Synthetic peptide
KCGSKDNIKH

SEQ ID NO: 461 - protein, 9 aa, synthetic construct
  REGION 1..9: note = Synthetic peptide
CGSKDNIKH

SEQ ID NO: 462 - protein, 8 aa, synthetic construct
  REGION 1..8: note = Synthetic peptide
GSKDNIKH

SEQ ID NO: 463 - protein, 7 aa, synthetic construct
  REGION 1..7: note = Synthetic peptide
SKDNIKH

SEQ ID NO: 464 - protein, 6 aa, synthetic construct
  REGION 1..6: note = Synthetic peptide
KDNIKH

SEQ ID NO: 465 - protein, 5 aa, synthetic construct
  REGION 1..5: note = Synthetic peptide
DNIKH

SEQ ID NO: 466 - protein, 4 aa, synthetic construct
  REGION 1..4: note = Synthetic peptide
NIKH

SEQ ID NO: 467 - skipped

SEQ ID NO: 468 - protein, 10 aa, synthetic construct
  REGION 1..10: note = Synthetic peptide
CGSKDNIKHV

SEQ ID NO: 469 - protein, 9 aa, synthetic construct
  REGION 1..9: note = Synthetic peptide
GSKDNIKHV

SEQ ID NO: 470 - protein, 8 aa, synthetic construct
  REGION 1..8: note = Synthetic peptide
SKDNIKHV

SEQ ID NO: 471 - protein, 7 aa, synthetic construct
  REGION 1..7: note = Synthetic peptide
KDNIKHV

SEQ ID NO: 472 - protein, 6 aa, synthetic construct
  REGION 1..6: note = Synthetic peptide
DNIKHV

SEQ ID NO: 473 - protein, 5 aa, synthetic construct
  REGION 1..5: note = Synthetic peptide
NIKHV

SEQ ID NO: 474 - protein, 4 aa, synthetic construct
  REGION 1..4: note = Synthetic peptide
IKHV

SEQ ID NO: 475 - skipped

SEQ ID NO: 476 - protein, 10 aa, synthetic construct
  REGION 1..10: note = Synthetic peptide
GSKDNIKHVP

SEQ ID NO: 477 - protein, 9 aa, synthetic construct
  REGION 1..9: note = Synthetic peptide
SKDNIKHVP

SEQ ID NO: 478 - protein, 8 aa, synthetic construct
  REGION 1..8: note = Synthetic peptide
KDNIKHVP

SEQ ID NO: 479 - protein, 7 aa, synthetic construct
  REGION 1..7: note = Synthetic peptide
DNIKHVP

SEQ ID NO: 480 - protein, 5 aa, synthetic construct
  REGION 1..5: note = Synthetic peptide
IKHVP

SEQ ID NO: 481 - protein, 4 aa, synthetic construct
  REGION 1..4: note = Synthetic peptide
KHVP

SEQ ID NO: 482 - skipped

SEQ ID NO: 483 - protein, 10 aa, synthetic construct
  REGION 1..10: note = Synthetic peptide
SKDNIKHVPG

SEQ ID NO: 484 - protein, 9 aa, synthetic construct
  REGION 1..9: note = Synthetic peptide
KDNIKHVPG

SEQ ID NO: 485 - protein, 8 aa, synthetic construct
  REGION 1..8: note = Synthetic peptide
DNIKHVPG

SEQ ID NO: 486 - protein, 7 aa, synthetic construct
  REGION 1..7: note = Synthetic peptide
NIKHVPG

SEQ ID NO: 487 - protein, 6 aa, synthetic construct
  REGION 1..6: note = Synthetic peptide
IKHVPG

SEQ ID NO: 488 - protein, 5 aa, synthetic construct
  REGION 1..5: note = Synthetic peptide
KHVPG

SEQ ID NO: 489 - protein, 4 aa, synthetic construct
  REGION 1..4: note = Synthetic peptide
HVPG

SEQ ID NO: 490 - skipped

SEQ ID NO: 491 - protein, 10 aa, synthetic construct
  REGION 1..10: note = Synthetic peptide
KDNIKHVPGG

SEQ ID NO: 492 - protein, 9 aa, synthetic construct
  REGION 1..9: note = Synthetic peptide
DNIKHVPGG

SEQ ID NO: 493 - protein, 8 aa, synthetic construct
  REGION 1..8: note = Synthetic peptide
NIKHVPGG

SEQ ID NO: 494 - protein, 7 aa, synthetic construct
  REGION 1..7: note = Synthetic peptide
IKHVPGG

SEQ ID NO: 495 - protein, 6 aa, synthetic construct
  REGION 1..6: note = Synthetic peptide
KHVPGG

SEQ ID NO: 496 - protein, 4 aa, synthetic construct
  REGION 1..4: note = Synthetic peptide
VPGG

SEQ ID NO: 497 - skipped

SEQ ID NO: 498 - protein, 10 aa, synthetic construct
  REGION 1..10: note = Synthetic peptide
DNIKHVPGGG

SEQ ID NO: 499 - protein, 9 aa, synthetic construct
  REGION 1..9: note = Synthetic peptide
NIKHVPGGG

SEQ ID NO: 500 - protein, 8 aa, synthetic construct
  REGION 1..8: note = Synthetic peptide
IKHVPGGG

SEQ ID NO: 501 - protein, 5 aa, synthetic construct
  REGION 1..5: note = Synthetic peptide
VPGGG

SEQ ID NO: 502 - protein, 4 aa, synthetic construct
  REGION 1..4: note = Synthetic peptide
PGGG

SEQ ID NO: 503 - skipped

SEQ ID NO: 504 - protein, 10 aa, synthetic construct
  REGION 1..10: note = Synthetic peptide
NIKHVPGGGS

SEQ ID NO: 505 - protein, 9 aa, synthetic construct
  REGION 1..9: note = Synthetic peptide
IKHVPGGGS

SEQ ID NO: 506 - protein, 8 aa, synthetic construct
  REGION 1..8: note = Synthetic peptide
KHVPGGGS

SEQ ID NO: 507 - protein, 7 aa, synthetic construct
  REGION 1..7: note = Synthetic peptide
HVPGGGS

SEQ ID NO: 508 - protein, 6 aa, synthetic construct
  REGION 1..6: note = Synthetic peptide
VPGGGS

SEQ ID NO: 509 - protein, 5 aa, synthetic construct
  REGION 1..5: note = Synthetic peptide
PGGGS

SEQ ID NO: 510 - protein, 4 aa, synthetic construct
  REGION 1..4: note = Synthetic peptide
GGGS

SEQ ID NO: 511 - skipped

SEQ ID NO: 512 - protein, 10 aa, synthetic construct
  REGION 1..10: note = Synthetic peptide
IKHVPGGGSV

SEQ ID NO: 513 - protein, 9 aa, synthetic construct
  REGION 1..9: note = Synthetic peptide
KHVPGGGSV

SEQ ID NO: 514 - protein, 8 aa, synthetic construct
  REGION 1..8: note = Synthetic peptide
HVPGGGSV

SEQ ID NO: 515 - protein, 7 aa, synthetic construct
  REGION 1..7: note = Synthetic peptide
VPGGGSV

SEQ ID NO: 516 - protein, 6 aa, synthetic construct
  REGION 1..6: note = Synthetic peptide
PGGGSV

SEQ ID NO: 517 - protein, 5 aa, synthetic construct
  REGION 1..5: note = Synthetic peptide
GGGSV

SEQ ID NO: 518 - protein, 4 aa, synthetic construct
  REGION 1..4: note = Synthetic peptide
GGSV

SEQ ID NO: 519 - skipped

SEQ ID NO: 520 - protein, 10 aa, synthetic construct
  REGION 1..10: note = Synthetic peptide
KHVPGGGSVQ

SEQ ID NO: 521 - protein, 9 aa, synthetic construct
  REGION 1..9: note = Synthetic peptide
HVPGGGSVQ

SEQ ID NO: 522 - protein, 8 aa, synthetic construct
  REGION 1..8: note = Synthetic peptide
VPGGGSVQ

SEQ ID NO: 523 - protein, 7 aa, synthetic construct
  REGION 1..7: note = Synthetic peptide
PGGGSVQ

SEQ ID NO: 524 - protein, 6 aa, synthetic construct
  REGION 1..6: note = Synthetic peptide
GGGSVQ

SEQ ID NO: 525 - protein, 5 aa, synthetic construct
  REGION 1..5: note = Synthetic peptide
GGSVQ

SEQ ID NO: 526 - protein, 4 aa, synthetic construct
  REGION 1..4: note = Synthetic peptide
GSVQ

SEQ ID NO: 527 - skipped

SEQ ID NO: 528 - protein, 10 aa, synthetic construct
  REGION 1..10: note = Synthetic peptide
HVPGGGSVQI

SEQ ID NO: 529 - protein, 9 aa, synthetic construct
  REGION 1..9: note = Synthetic peptide
VPGGGSVQI

SEQ ID NO: 530 - protein, 8 aa, synthetic construct
  REGION 1..8: note = Synthetic peptide
PGGGSVQI

SEQ ID NO: 531 - protein, 7 aa, synthetic construct
  REGION 1..7: note = Synthetic peptide
GGGSVQI

SEQ ID NO: 532 - protein, 6 aa, synthetic construct
  REGION 1..6: note = Synthetic peptide
GGSVQI

SEQ ID NO: 533 - protein, 5 aa, synthetic construct
  REGION 1..5: note = Synthetic peptide
GSVQI

SEQ ID NO: 534 - protein, 4 aa, synthetic construct
  REGION 1..4: note = Synthetic peptide
SVQI

SEQ ID NO: 535 - protein, 10 aa, synthetic construct
  REGION 1..10: note = Synthetic peptide
GGSVQIVYKS

SEQ ID NO: 536 - protein, 9 aa, synthetic construct
  REGION 1..9: note = Synthetic peptide
GSVQIVYKS

SEQ ID NO: 537 - protein, 8 aa, synthetic construct
  REGION 1..8: note = Synthetic peptide
SVQIVYKS

SEQ ID NO: 538 - protein, 7 aa, synthetic construct
  REGION 1..7: note = Synthetic peptide
VQIVYKS

SEQ ID NO: 539 - protein, 6 aa, synthetic construct
  REGION 1..6: note = Synthetic peptide
QIVYKS

SEQ ID NO: 540 - protein, 5 aa, synthetic construct
  REGION 1..5: note = Synthetic peptide
IVYKS

SEQ ID NO: 541 - protein, 4 aa, synthetic construct
  REGION 1..4: note = Synthetic peptide
VYKS

SEQ ID NO: 542 - skipped

SEQ ID NO: 543 - protein, 10 aa, synthetic construct
  REGION 1..10: note = Synthetic peptide
GSVQIVYKSV

SEQ ID NO: 544 - protein, 9 aa, synthetic construct
  REGION 1..9: note = Synthetic peptide
SVQIVYKSV

SEQ ID NO: 545 - protein, 8 aa, synthetic construct
  REGION 1..8: note = Synthetic peptide
VQIVYKSV

SEQ ID NO: 546 - protein, 7 aa, synthetic construct
  REGION 1..7: note = Synthetic peptide
QIVYKSV

SEQ ID NO: 547 - protein, 6 aa, synthetic construct
  REGION 1..6: note = Synthetic peptide
IVYKSV

SEQ ID NO: 548 - protein, 5 aa, synthetic construct
  REGION 1..5: note = Synthetic peptide
VYKSV

SEQ ID NO: 549 - protein, 4 aa, synthetic construct
  REGION 1..4: note = Synthetic peptide
YKSV

SEQ ID NO: 550 - protein, 10 aa, synthetic construct
  REGION 1..10: note = Synthetic peptide
SVQIVYKSVD

SEQ ID NO: 551 - protein, 9 aa, synthetic construct
  REGION 1..9: note = Synthetic peptide
VQIVYKSVD

SEQ ID NO: 552 - protein, 8 aa, synthetic construct
  REGION 1..8: note = Synthetic peptide
QIVYKSVD

SEQ ID NO: 553 - protein, 7 aa, synthetic construct
  REGION 1..7: note = Synthetic peptide
IVYKSVD

SEQ ID NO: 554 - protein, 6 aa, synthetic construct
  REGION 1..6: note = Synthetic peptide
VYKSVD

SEQ ID NO: 555 - protein, 5 aa, synthetic construct
  REGION 1..5: note = Synthetic peptide
YKSVD

SEQ ID NO: 556 - protein, 4 aa, synthetic construct
  REGION 1..4: note = Synthetic peptide
KSVD

SEQ ID NO: 557 - skipped

SEQ ID NO: 558 - protein, 10 aa, synthetic construct
  REGION 1..10: note = Synthetic peptide
VQIVYKSVDL

SEQ ID NO: 559 - protein, 9 aa, synthetic construct
  REGION 1..9: note = Synthetic peptide
QIVYKSVDL

SEQ ID NO: 560 - protein, 8 aa, synthetic construct
  REGION 1..8: note = Synthetic peptide
IVYKSVDL

SEQ ID NO: 561 - protein, 7 aa, synthetic construct
  REGION 1..7: note = Synthetic peptide
VYKSVDL

SEQ ID NO: 562 - protein, 6 aa, synthetic construct
  REGION 1..6: note = Synthetic peptide
YKSVDL

SEQ ID NO: 563 - protein, 5 aa, synthetic construct
  REGION 1..5: note = Synthetic peptide
KSVDL

SEQ ID NO: 564 - protein, 4 aa, synthetic construct
  REGION 1..4: note = Synthetic peptide
SVDL

SEQ ID NO: 565 - protein, 10 aa, synthetic construct
  REGION 1..10: note = Synthetic peptide
QIVYKSVDLS

SEQ ID NO: 566 - protein, 9 aa, synthetic construct
  REGION 1..9: note = Synthetic peptide
IVYKSVDLS

SEQ ID NO: 567 - protein, 8 aa, synthetic construct
  REGION 1..8: note = Synthetic peptide
VYKSVDLS

SEQ ID NO: 568 - protein, 7 aa, synthetic construct
  REGION 1..7: note = Synthetic peptide
YKSVDLS

SEQ ID NO: 569 - protein, 6 aa, synthetic construct
  REGION 1..6: note = Synthetic peptide
KSVDLS

SEQ ID NO: 570 - protein, 5 aa, synthetic construct
  REGION 1..5: note = Synthetic peptide
SVDLS

SEQ ID NO: 571 - protein, 10 aa, synthetic construct
  REGION 1..10: note = Synthetic peptide
IVYKSVDLSK

SEQ ID NO: 572 - protein, 9 aa, synthetic construct
  REGION 1..9: note = Synthetic peptide
VYKSVDLSK

SEQ ID NO: 573 - protein, 8 aa, synthetic construct
  REGION 1..8: note = Synthetic peptide
YKSVDLSK

SEQ ID NO: 574 - protein, 7 aa, synthetic construct
  REGION 1..7: note = Synthetic peptide
KSVDLSK

SEQ ID NO: 575 - protein, 10 aa, synthetic construct
  REGION 1..10: note = Synthetic peptide
VYKSVDLSKV

SEQ ID NO: 576 - protein, 9 aa, synthetic construct
  REGION 1..9: note = Synthetic peptide
YKSVDLSKV

SEQ ID NO: 577 - protein, 8 aa, synthetic construct
  REGION 1..8: note = Synthetic peptide
KSVDLSKV

SEQ ID NO: 578 - protein, 7 aa, synthetic construct
  REGION 1..7: note = Synthetic peptide
SVDLSKV

SEQ ID NO: 579 - protein, 10 aa, synthetic construct
  REGION 1..10: note = Synthetic peptide
YKSVDLSKVT

SEQ ID NO: 580 - protein, 9 aa, synthetic construct
  REGION 1..9: note = Synthetic peptide
KSVDLSKVT

SEQ ID NO: 581 - protein, 8 aa, synthetic construct
  REGION 1..8: note = Synthetic peptide
SVDLSKVT

SEQ ID NO: 582 - protein, 6 aa, synthetic construct
  REGION 1..6: note = Synthetic peptide
DLSKVT

SEQ ID NO: 583 - protein, 5 aa, synthetic construct
  REGION 1..5: note = Synthetic peptide
LSKVT

SEQ ID NO: 584 - protein, 4 aa, synthetic construct
  REGION 1..4: note = Synthetic peptide
SKVT

SEQ ID NO: 585 - skipped

SEQ ID NO: 586 - protein, 10 aa, synthetic construct
  REGION 1..10: note = Synthetic peptide
HGAEIVYKSV

SEQ ID NO: 587 - protein, 9 aa, synthetic construct
  REGION 1..9: note = Synthetic peptide
GAEIVYKSV

SEQ ID NO: 588 - protein, 8 aa, synthetic construct
  REGION 1..8: note = Synthetic peptide
AEIVYKSV

SEQ ID NO: 589 - protein, 10 aa, synthetic construct
  REGION 1..10: note = Synthetic peptide
GAEIVYKSVV

SEQ ID NO: 590 - protein, 9 aa, synthetic construct
  REGION 1..9: note = Synthetic peptide
AEIVYKSVV

SEQ ID NO: 591 - protein, 8 aa, synthetic construct
  REGION 1..8: note = Synthetic peptide
EIVYKSVV

SEQ ID NO: 592 - protein, 7 aa, synthetic construct
  REGION 1..7: note = Synthetic peptide
IVYKSVV

SEQ ID NO: 593 - protein, 6 aa, synthetic construct
  REGION 1..6: note = Synthetic peptide
VYKSVV

SEQ ID NO: 594 - protein, 5 aa, synthetic construct
  REGION 1..5: note = Synthetic peptide
YKSVV

SEQ ID NO: 595 - protein, 4 aa, synthetic construct
  REGION 1..4: note = Synthetic peptide
KSVV

SEQ ID NO: 596 - skipped

SEQ ID NO: 597 - protein, 10 aa, synthetic construct
  REGION 1..10: note = Synthetic peptide
AEIVYKSVVS

SEQ ID NO: 598 - protein, 9 aa, synthetic construct
  REGION 1..9: note = Synthetic peptide
EIVYKSVVS

SEQ ID NO: 599 - protein, 8 aa, synthetic construct
  REGION 1..8: note = Synthetic peptide
IVYKSVVS

SEQ ID NO: 600 - protein, 7 aa, synthetic construct
  REGION 1..7: note = Synthetic peptide
VYKSVVS

SEQ ID NO: 601 - protein, 6 aa, synthetic construct
  REGION 1..6: note = Synthetic peptide
YKSVVS

SEQ ID NO: 602 - protein, 5 aa, synthetic construct
  REGION 1..5: note = Synthetic peptide
KSVVS

SEQ ID NO: 603 - protein, 4 aa, synthetic construct
  REGION 1..4: note = Synthetic peptide
SVVS

SEQ ID NO: 604 - protein, 10 aa, synthetic construct
  REGION 1..10: note = Synthetic peptide
EIVYKSVVSG

SEQ ID NO: 605 - protein, 9 aa, synthetic construct
  REGION 1..9: note = Synthetic peptide
IVYKSVVSG

SEQ ID NO: 606 - protein, 8 aa, synthetic construct
  REGION 1..8: note = Synthetic peptide
VYKSVVSG

SEQ ID NO: 607 - protein, 7 aa, synthetic construct
  REGION 1..7: note = Synthetic peptide
YKSVVSG

SEQ ID NO: 608 - protein, 6 aa, synthetic construct
  REGION 1..6: note = Synthetic peptide
KSVVSG

SEQ ID NO: 609 - protein, 5 aa, synthetic construct
  REGION 1..5: note = Synthetic peptide
SVVSG

SEQ ID NO: 610 - protein, 4 aa, synthetic construct
  REGION 1..4: note = Synthetic peptide
VVSG

SEQ ID NO: 611 - skipped

SEQ ID NO: 612 - protein, 10 aa, synthetic construct
  REGION 1..10: note = Synthetic peptide
IVYKSVVSGD

SEQ ID NO: 613 - protein, 9 aa, synthetic construct
  REGION 1..9: note = Synthetic peptide
VYKSVVSGD

SEQ ID NO: 614 - protein, 8 aa, synthetic construct
  REGION 1..8: note = Synthetic peptide
YKSVVSGD

SEQ ID NO: 615 - protein, 7 aa, synthetic construct
  REGION 1..7: note = Synthetic peptide
KSVVSGD

SEQ ID NO: 616 - protein, 6 aa, synthetic construct
  REGION 1..6: note = Synthetic peptide
SVVSGD

SEQ ID NO: 617 - protein, 5 aa, synthetic construct
  REGION 1..5: note = Synthetic peptide
VVSGD

SEQ ID NO: 618 - protein, 10 aa, synthetic construct
  REGION 1..10: note = Synthetic peptide
VYKSVVSGDT

SEQ ID NO: 619 - protein, 9 aa, synthetic construct
  REGION 1..9: note = Synthetic peptide
YKSVVSGDT

SEQ ID NO: 620 - protein, 8 aa, synthetic construct
  REGION 1..8: note = Synthetic peptide
KSVVSGDT

SEQ ID NO: 621 - protein, 7 aa, synthetic construct
  REGION 1..7: note = Synthetic peptide
SVVSGDT

SEQ ID NO: 622 - protein, 10 aa, synthetic construct
  REGION 1..10: note = Synthetic peptide
YKSVVSGDTS

SEQ ID NO: 623 - protein, 9 aa, synthetic construct
  REGION 1..9: note = Synthetic peptide
KSVVSGDTS

SEQ ID NO: 624 - protein, 8 aa, synthetic construct
  REGION 1..8: note = Synthetic peptide
SVVSGDTS

SEQ ID NO: 625 - protein, 10 aa, synthetic construct
  REGION 1..10: note = Synthetic peptide
YKSVVSGDTS

SEQ ID NO: 626 - protein, 9 aa, synthetic construct
  REGION 1..9: note = Synthetic peptide
KSVVSGDTS

SEQ ID NO: 627 - protein, 8 aa, synthetic construct
  REGION 1..8: note = Synthetic peptide
SVVSGDTS

SEQ ID NO: 628 - protein, 7 aa, synthetic construct
  REGION 1..7: note = Synthetic peptide
VVSGDTS

SEQ ID NO: 629 - protein, 10 aa, synthetic construct
  REGION 1..10: note = Synthetic peptide
KSVVSGDTSP

SEQ ID NO: 630 - protein, 9 aa, synthetic construct
  REGION 1..9: note = Synthetic peptide
SVVSGDTSP

SEQ ID NO: 631 - protein, 10 aa, synthetic construct
  REGION 1..10: note = Synthetic peptide
SVVSGDTSPR

SEQ ID NO: 632 - protein, 10 aa, synthetic construct
  REGION 1..10: note = Synthetic peptide
DHGAEIVYKP

SEQ ID NO: 633 - protein, 9 aa, synthetic construct
  REGION 1..9: note = Synthetic peptide
HGAEIVYKP

SEQ ID NO: 634 - protein, 8 aa, synthetic construct
  REGION 1..8: note = Synthetic peptide
GAEIVYKP

SEQ ID NO: 635 - protein, 7 aa, synthetic construct
  REGION 1..7: note = Synthetic peptide
AEIVYKP

SEQ ID NO: 636 - protein, 10 aa, synthetic construct
  REGION 1..10: note = Synthetic peptide
HGAEIVYKPV

SEQ ID NO: 637 - protein, 9 aa, synthetic construct
  REGION 1..9: note = Synthetic peptide
GAEIVYKPV

SEQ ID NO: 638 - protein, 8 aa, synthetic construct
  REGION 1..8: note = Synthetic peptide
AEIVYKPV

SEQ ID NO: 639 - protein, 10 aa, synthetic construct
  REGION 1..10: note = Synthetic peptide
GAEIVYKPVV

SEQ ID NO: 640 - protein, 9 aa, synthetic construct
  REGION 1..9: note = Synthetic peptide
AEIVYKPVV

SEQ ID NO: 641 - protein, 8 aa, synthetic construct
  REGION 1..8: note = Synthetic peptide
EIVYKPVV

SEQ ID NO: 642 - protein, 7 aa, synthetic construct
  REGION 1..7: note = Synthetic peptide
IVYKPVV

SEQ ID NO: 643 - protein, 6 aa, synthetic construct
  REGION 1..6: note = Synthetic peptide
VYKPVV

SEQ ID NO: 644 - protein, 5 aa, synthetic construct
  REGION 1..5: note = Synthetic peptide
YKPVV

SEQ ID NO: 645 - protein, 4 aa, synthetic construct
  REGION 1..4: note = Synthetic peptide
KPVV

SEQ ID NO: 646 - protein, 10 aa, synthetic construct
  REGION 1..10: note = Synthetic peptide
AEIVYKPVVS

SEQ ID NO: 647 - protein, 9 aa, synthetic construct
  REGION 1..9: note = Synthetic peptide
EIVYKPVVS

SEQ ID NO: 648 - protein, 8 aa, synthetic construct
  REGION 1..8: note = Synthetic peptide
IVYKPVVS

SEQ ID NO: 649 - protein, 7 aa, synthetic construct
  REGION 1..7: note = Synthetic peptide
VYKPVVS

SEQ ID NO: 650 - protein, 6 aa, synthetic construct
  REGION 1..6: note = Synthetic peptide
YKPVVS

SEQ ID NO: 651 - protein, 5 aa, synthetic construct
  REGION 1..5: note = Synthetic peptide
KPVVS

SEQ ID NO: 652 - protein, 10 aa, synthetic construct
  REGION 1..10: note = Synthetic peptide
EIVYKPVVSG

SEQ ID NO: 653 - protein, 9 aa, synthetic construct
  REGION 1..9: note = Synthetic peptide
IVYKPVVSG

SEQ ID NO: 654 - protein, 8 aa, synthetic construct
  REGION 1..8: note = Synthetic peptide
VYKPVVSG

SEQ ID NO: 655 - protein, 7 aa, synthetic construct
  REGION 1..7: note = Synthetic peptide
YKPVVSG

SEQ ID NO: 656 - protein, 6 aa, synthetic construct
  REGION 1..6: note = Synthetic peptide
KPVVSG

SEQ ID NO: 657 - protein, 10 aa, synthetic construct
  REGION 1..10: note = Synthetic peptide
IVYKPVVSGD

SEQ ID NO: 658 - protein, 9 aa, synthetic construct
  REGION 1..9: note = Synthetic peptide
VYKPVVSGD

SEQ ID NO: 659 - protein, 8 aa, synthetic construct
  REGION 1..8: note = Synthetic peptide
YKPVVSGD

SEQ ID NO: 660 - protein, 7 aa, synthetic construct
  REGION 1..7: note = Synthetic peptide
KPVVSGD

SEQ ID NO: 661 - protein, 10 aa, synthetic construct
  REGION 1..10: note = Synthetic peptide
VYKPVVSGDT

SEQ ID NO: 662 - protein, 9 aa, synthetic construct
  REGION 1..9: note = Synthetic peptide
YKPVVSGDT

SEQ ID NO: 663 - protein, 8 aa, synthetic construct
  REGION 1..8: note = Synthetic peptide
KPVVSGDT

SEQ ID NO: 664 - protein, 10 aa, synthetic construct
  REGION 1..10: note = Synthetic peptide
YKPVVSGDTS

SEQ ID NO: 665 - protein, 9 aa, synthetic construct
  REGION 1..9: note = Synthetic peptide
KPVVSGDTS

SEQ ID NO: 666 - protein, 8 aa, synthetic construct
  REGION 1..8: note = Synthetic peptide
PVVSGDTS

SEQ ID NO: 667 - protein, 7 aa, synthetic construct
  REGION 1..7: note = Synthetic peptide
VVSGDTS

SEQ ID NO: 668 - protein, 10 aa, synthetic construct
  REGION 1..10: note = Synthetic peptide
KPVVSGDTSP

SEQ ID NO: 669 - protein, 4 aa, synthetic construct
  REGION 1..4: note = Synthetic peptide
CNIK

SEQ ID NO: 670 - protein, 5 aa, synthetic construct
  REGION 1..5: note = Synthetic peptide
CNIKH

SEQ ID NO: 671 - protein, 6 aa, synthetic construct
  REGION 1..6: note = Synthetic peptide
CNIKHV

SEQ ID NO: 672 - protein, 9 aa, synthetic construct
  REGION 1..9: note = Synthetic peptide
CNIKHVPGG

SEQ ID NO: 673 - protein, 10 aa, synthetic construct
  REGION 1..10: note = Synthetic peptide
CNIKHVPGGG

SEQ ID NO: 674 - protein, 11 aa, synthetic construct
  REGION 1..11: note = Synthetic peptide
CNIKHVPGGGS

SEQ ID NO: 675 - protein, 10 aa, synthetic construct
  REGION 1..10: note = Synthetic peptide
ENLKHQPGGG

SEQ ID NO: 676 - protein, 9 aa, synthetic construct
  REGION 1..9: note = Synthetic peptide
NLKHQPGGG

SEQ ID NO: 677 - protein, 8 aa, synthetic construct
  REGION 1..8: note = Synthetic peptide
LKHQPGGG

SEQ ID NO: 678 - protein, 7 aa, synthetic construct
  REGION 1..7: note = Synthetic peptide
KHQPGGG

SEQ ID NO: 679 - protein, 6 aa, synthetic construct
  REGION 1..6: note = Synthetic peptide
HQPGGG

SEQ ID NO: 680 - protein, 5 aa, synthetic construct
  REGION 1..5: note = Synthetic peptide
QPGGG

SEQ ID NO: 681 - protein, 10 aa, synthetic construct
  REGION 1..10: note = Synthetic peptide
TENLKHQPGG

SEQ ID NO: 682 - protein, 9 aa, synthetic construct
  REGION 1..9: note = Synthetic peptide
ENLKHQPGG

SEQ ID NO: 683 - protein, 8 aa, synthetic construct
  REGION 1..8: note = Synthetic peptide
NLKHQPGG

SEQ ID NO: 684 - protein, 7 aa, synthetic construct
  REGION 1..7: note = Synthetic peptide
LKHQPGG

SEQ ID NO: 685 - protein, 6 aa, synthetic construct
  REGION 1..6: note = Synthetic peptide
KHQPGG

SEQ ID NO: 686 - protein, 5 aa, synthetic construct
  REGION 1..5: note = Synthetic peptide
HQPGG

SEQ ID NO: 687 - protein, 4 aa, synthetic construct
  REGION 1..4: note = Synthetic peptide
QPGG

SEQ ID NO: 688 - protein, 9 aa, synthetic construct
  REGION 1..9: note = Synthetic peptide
TENLKHQPG

SEQ ID NO: 689 - protein, 8 aa, synthetic construct
  REGION 1..8: note = Synthetic peptide
ENLKHQPG

SEQ ID NO: 690 - protein, 7 aa, synthetic construct
  REGION 1..7: note = Synthetic peptide
NLKHQPG

SEQ ID NO: 691 - protein, 6 aa, synthetic construct
  REGION 1..6: note = Synthetic peptide
LKHQPG

SEQ ID NO: 692 - protein, 5 aa, synthetic construct
  REGION 1..5: note = Synthetic peptide
KHQPG

SEQ ID NO: 693 - protein, 4 aa, synthetic construct
  REGION 1..4: note = Synthetic peptide
HQPG

SEQ ID NO: 694 - skipped

SEQ ID NO: 695 - protein, 8 aa, synthetic construct
  REGION 1..8: note = Synthetic peptide
TENLKHQP

SEQ ID NO: 696 - protein, 7 aa, synthetic construct
  REGION 1..7: note = Synthetic peptide
ENLKHQP

SEQ ID NO: 697 - protein, 6 aa, synthetic construct
  REGION 1..6: note = Synthetic peptide
NLKHQP

SEQ ID NO: 698 - protein, 5 aa, synthetic construct
  REGION 1..5: note = Synthetic peptide
LKHQP

SEQ ID NO: 699 - protein, 4 aa, synthetic construct
  REGION 1..4: note = Synthetic peptide
KHQP

SEQ ID NO: 700 - skipped

SEQ ID NO: 701 - protein, 7 aa, synthetic construct
  REGION 1..7: note = Synthetic peptide
TENLKHQ

SEQ ID NO: 702 - protein, 6 aa, synthetic construct
  REGION 1..6: note = Synthetic peptide
ENLKHQ

SEQ ID NO: 703 - protein, 5 aa, synthetic construct
  REGION 1..5: note = Synthetic peptide
NLKHQ

SEQ ID NO: 704 - protein, 4 aa, synthetic construct
  REGION 1..4: note = Synthetic peptide
LKHQ

SEQ ID NO: 705 - skipped

SEQ ID NO: 706 - protein, 6 aa, synthetic construct
  REGION 1..6: note = Synthetic peptide
TENLKH

SEQ ID NO: 707 - protein, 5 aa, synthetic construct
  REGION 1..5: note = Synthetic peptide
ENLKH

SEQ ID NO: 708 - protein, 4 aa, synthetic construct
  REGION 1..4: note = Synthetic peptide
NLKH

SEQ ID NO: 709 - skipped

SEQ ID NO: 710 - protein, 5 aa, synthetic construct
  REGION 1..5: note = Synthetic peptide
TENLK

SEQ ID NO: 711 - protein, 4 aa, synthetic construct
  REGION 1..4: note = Synthetic peptide
ENLK

SEQ ID NO: 712 - skipped

SEQ ID NO: 713 - protein, 4 aa, synthetic construct
  REGION 1..4: note = Synthetic peptide
TENL

SEQ ID NO: 714-715 - skipped (2 entries)

SEQ ID NO: 716 - protein, 11 aa, synthetic construct
  REGION 1..11: note = Synthetic peptide
KDNIKHVPGGG

SEQ ID NO: 717 - protein, 4 aa, synthetic construct
  REGION 1..4: note = Synthetic peptide
KDNI

SEQ ID NO: 718 - skipped

SEQ ID NO: 719 - protein, 7 aa, synthetic construct
  REGION 1..7: note = Synthetic peptide
IKHVGGG

SEQ ID NO: 720 - protein, 6 aa, synthetic construct
  REGION 1..6: note = Synthetic peptide
IKHVGG

SEQ ID NO: 721 - protein, 5 aa, synthetic construct
  REGION 1..5: note = Synthetic peptide
IKHVG

SEQ ID NO: 722 - protein, 6 aa, synthetic construct
  REGION 1..6: note = Synthetic peptide
KHVGGG

SEQ ID NO: 723 - protein, 5 aa, synthetic construct
  REGION 1..5: note = Synthetic peptide
KHVGG

SEQ ID NO: 724 - protein, 4 aa, synthetic construct
  REGION 1..4: note = Synthetic peptide
KHVG

SEQ ID NO: 725 - protein, 11 aa, synthetic construct
  REGION 1..11: note = Synthetic peptide
LGNIHHKPGGG

SEQ ID NO: 726 - protein, 10 aa, synthetic construct
  REGION 1..10: note = Synthetic peptide
GNIHHKPGGG

SEQ ID NO: 727 - protein, 9 aa, synthetic construct
  REGION 1..9: note = Synthetic peptide
NIHHKPGGG

SEQ ID NO: 728 - protein, 8 aa, synthetic construct
  REGION 1..8: note = Synthetic peptide
IHHKPGGG

SEQ ID NO: 729 - protein, 7 aa, synthetic construct
  REGION 1..7: note = Synthetic peptide
HHKPGGG

SEQ ID NO: 730 - protein, 5 aa, synthetic construct
  REGION 1..5: note = Synthetic peptide
KPGGG

SEQ ID NO: 731 - protein, 10 aa, synthetic construct
  REGION 1..10: note = Synthetic peptide
LGNIHHKPGG

SEQ ID NO: 732 - protein, 9 aa, synthetic construct
  REGION 1..9: note = Synthetic peptide
GNIHHKPGG

SEQ ID NO: 733 - protein, 8 aa, synthetic construct
  REGION 1..8: note = Synthetic peptide
NIHHKPGG

SEQ ID NO: 734 - protein, 7 aa, synthetic construct
  REGION 1..7: note = Synthetic peptide
IHHKPGG

SEQ ID NO: 735 - protein, 6 aa, synthetic construct
  REGION 1..6: note = Synthetic peptide
HHKPGG

SEQ ID NO: 736 - protein, 4 aa, synthetic construct
  REGION 1..4: note = Synthetic peptide
KPGG

SEQ ID NO: 737 - protein, 9 aa, synthetic construct
  REGION 1..9: note = Synthetic peptide
LGNIHHKPG

SEQ ID NO: 738 - protein, 8 aa, synthetic construct
  REGION 1..8: note = Synthetic peptide
GNIHHKPG

SEQ ID NO: 739 - protein, 7 aa, synthetic construct
  REGION 1..7: note = Synthetic peptide
NIHHKPG

SEQ ID NO: 740 - protein, 6 aa, synthetic construct
  REGION 1..6: note = Synthetic peptide
IHHKPG

SEQ ID NO: 741 - protein, 5 aa, synthetic construct
  REGION 1..5: note = Synthetic peptide
HHKPG

SEQ ID NO: 742 - protein, 4 aa, synthetic construct
  REGION 1..4: note = Synthetic peptide
HKPG

SEQ ID NO: 743 - skipped

SEQ ID NO: 744 - protein, 8 aa, synthetic construct
  REGION 1..8: note = Synthetic peptide
LGNIHHKP

SEQ ID NO: 745 - protein, 7 aa, synthetic construct
  REGION 1..7: note = Synthetic peptide
GNIHHKP

SEQ ID NO: 746 - protein, 6 aa, synthetic construct
  REGION 1..6: note = Synthetic peptide
NIHHKP

SEQ ID NO: 747 - protein, 5 aa, synthetic construct
  REGION 1..5: note = Synthetic peptide
IHHKP

SEQ ID NO: 748 - protein, 4 aa, synthetic construct
  REGION 1..4: note = Synthetic peptide
HHKP

SEQ ID NO: 749 - skipped

SEQ ID NO: 750 - protein, 7 aa, synthetic construct
  REGION 1..7: note = Synthetic peptide
LGNIHHK

SEQ ID NO: 751 - protein, 6 aa, synthetic construct
  REGION 1..6: note = Synthetic peptide
GNIHHK

SEQ ID NO: 752 - protein, 5 aa, synthetic construct
  REGION 1..5: note = Synthetic peptide
NIHHK

SEQ ID NO: 753 - protein, 4 aa, synthetic construct
  REGION 1..4: note = Synthetic peptide
IHHK

SEQ ID NO: 754 - skipped

SEQ ID NO: 755 - protein, 6 aa, synthetic construct
  REGION 1..6: note = Synthetic peptide
LGNIHH

SEQ ID NO: 756 - protein, 5 aa, synthetic construct
  REGION 1..5: note = Synthetic peptide
GNIHH

SEQ ID NO: 757 - protein, 4 aa, synthetic construct
  REGION 1..4: note = Synthetic peptide
NIHH

SEQ ID NO: 758 - skipped

SEQ ID NO: 759 - protein, 5 aa, synthetic construct
  REGION 1..5: note = Synthetic peptide
LGNIH

SEQ ID NO: 760 - protein, 4 aa, synthetic construct
  REGION 1..4: note = Synthetic peptide
GNIH

SEQ ID NO: 761 - skipped

SEQ ID NO: 762 - protein, 4 aa, synthetic construct
  REGION 1..4: note = Synthetic peptide
LGNI

SEQ ID NO: 763-764 - skipped (2 entries)

SEQ ID NO: 765 - protein, 11 aa, synthetic construct
  REGION 1..11: note = Synthetic peptide
LDNITHVPGGG

SEQ ID NO: 766 - protein, 10 aa, synthetic construct
  REGION 1..10: note = Synthetic peptide
DNITHVPGGG

SEQ ID NO: 767 - protein, 9 aa, synthetic construct
  REGION 1..9: note = Synthetic peptide
NITHVPGGG

SEQ ID NO: 768 - protein, 8 aa, synthetic construct
  REGION 1..8: note = Synthetic peptide
ITHVPGGG

SEQ ID NO: 769 - protein, 7 aa, synthetic construct
  REGION 1..7: note = Synthetic peptide
THVPGGG

SEQ ID NO: 770 - protein, 10 aa, synthetic construct
  REGION 1..10: note = Synthetic peptide
LDNITHVPGG

SEQ ID NO: 771 - protein, 9 aa, synthetic construct
  REGION 1..9: note = Synthetic peptide
DNITHVPGG

SEQ ID NO: 772 - protein, 8 aa, synthetic construct
  REGION 1..8: note = Synthetic peptide
NITHVPGG

SEQ ID NO: 773 - protein, 7 aa, synthetic construct
  REGION 1..7: note = Synthetic peptide
ITHVPGG

SEQ ID NO: 774 - protein, 6 aa, synthetic construct
  REGION 1..6: note = Synthetic peptide
THVPGG

SEQ ID NO: 775 - protein, 9 aa, synthetic construct
  REGION 1..9: note = Synthetic peptide
LDNITHVPG

SEQ ID NO: 776 - protein, 8 aa, synthetic construct
  REGION 1..8: note = Synthetic peptide
DNITHVPG

SEQ ID NO: 777 - protein, 7 aa, synthetic construct
  REGION 1..7: note = Synthetic peptide
NITHVPG

SEQ ID NO: 778 - protein, 6 aa, synthetic construct
  REGION 1..6: note = Synthetic peptide
ITHVPG

SEQ ID NO: 779 - protein, 5 aa, synthetic construct
  REGION 1..5: note = Synthetic peptide
THVPG

SEQ ID NO: 780 - protein, 8 aa, synthetic construct
  REGION 1..8: note = Synthetic peptide
LDNITHVP

SEQ ID NO: 781 - protein, 7 aa, synthetic construct
  REGION 1..7: note = Synthetic peptide
DNITHVP

SEQ ID NO: 782 - protein, 6 aa, synthetic construct
  REGION 1..6: note = Synthetic peptide
NITHVP

SEQ ID NO: 783 - protein, 5 aa, synthetic construct
  REGION 1..5: note = Synthetic peptide
ITHVP

SEQ ID NO: 784 - protein, 4 aa, synthetic construct
  REGION 1..4: note = Synthetic peptide
THVP

SEQ ID NO: 785 - protein, 7 aa, synthetic construct
  REGION 1..7: note = Synthetic peptide
LDNITHV

SEQ ID NO: 786 - protein, 6 aa, synthetic construct
  REGION 1..6: note = Synthetic peptide
DNITHV

SEQ ID NO: 787 - protein, 5 aa, synthetic construct
  REGION 1..5: note = Synthetic peptide
NITHV

SEQ ID NO: 788 - protein, 4 aa, synthetic construct
  REGION 1..4: note = Synthetic peptide
ITHV

SEQ ID NO: 789 - skipped

SEQ ID NO: 790 - protein, 6 aa, synthetic construct
  REGION 1..6: note = Synthetic peptide
LDNITH

SEQ ID NO: 791 - protein, 5 aa, synthetic construct
  REGION 1..5: note = Synthetic peptide
DNITH

SEQ ID NO: 792 - protein, 4 aa, synthetic construct
  REGION 1..4: note = Synthetic peptide
NITH

SEQ ID NO: 793 - skipped

SEQ ID NO: 794 - protein, 5 aa, synthetic construct
  REGION 1..5: note = Synthetic peptide
LDNIT

SEQ ID NO: 795 - protein, 4 aa, synthetic construct
  REGION 1..4: note = Synthetic peptide
DNIT

SEQ ID NO: 796 - skipped

SEQ ID NO: 797 - protein, 4 aa, synthetic construct
  REGION 1..4: note = Synthetic peptide
LDNI

SEQ ID NO: 798 - skipped

SEQ ID NO: 799 - protein, 10 aa, synthetic construct
  REGION 1..10: note = Synthetic peptide
KNVKSKIGST

SEQ ID NO: 800 - protein, 9 aa, synthetic construct
  REGION 1..9: note = Synthetic peptide
NVKSKIGST

SEQ ID NO: 801 - protein, 8 aa, synthetic construct
  REGION 1..8: note = Synthetic peptide
VKSKIGST

SEQ ID NO: 802 - protein, 7 aa, synthetic construct
  REGION 1..7: note = Synthetic peptide
KSKIGST

SEQ ID NO: 803 - protein, 6 aa, synthetic construct
  REGION 1..6: note = Synthetic peptide
SKIGST

SEQ ID NO: 804 - protein, 5 aa, synthetic construct
  REGION 1..5: note = Synthetic peptide
KIGST

SEQ ID NO: 805 - protein, 4 aa, synthetic construct
  REGION 1..4: note = Synthetic peptide
IGST

SEQ ID NO: 806 - skipped

SEQ ID NO: 807 - protein, 10 aa, synthetic construct
  REGION 1..10: note = Synthetic peptide
NVKSKIGSTE

SEQ ID NO: 808 - protein, 9 aa, synthetic construct
  REGION 1..9: note = Synthetic peptide
VKSKIGSTE

SEQ ID NO: 809 - protein, 8 aa, synthetic construct
  REGION 1..8: note = Synthetic peptide
KSKIGSTE

SEQ ID NO: 810 - protein, 7 aa, synthetic construct
  REGION 1..7: note = Synthetic peptide
SKIGSTE

SEQ ID NO: 811 - protein, 6 aa, synthetic construct
  REGION 1..6: note = Synthetic peptide
KIGSTE

SEQ ID NO: 812 - protein, 5 aa, synthetic construct
  REGION 1..5: note = Synthetic peptide
IGSTE

SEQ ID NO: 813 - protein, 4 aa, synthetic construct
  REGION 1..4: note = Synthetic peptide
GSTE

SEQ ID NO: 814 - skipped

SEQ ID NO: 815 - protein, 10 aa, synthetic construct
  REGION 1..10: note = Synthetic peptide
VKSKIGSTEN

SEQ ID NO: 816 - protein, 9 aa, synthetic construct
  REGION 1..9: note = Synthetic peptide
KSKIGSTEN

SEQ ID NO: 817 - protein, 8 aa, synthetic construct
  REGION 1..8: note = Synthetic peptide
SKIGSTEN

SEQ ID NO: 818 - protein, 7 aa, synthetic construct
  REGION 1..7: note = Synthetic peptide
KIGSTEN

SEQ ID NO: 819 - protein, 6 aa, synthetic construct
  REGION 1..6: note = Synthetic peptide
IGSTEN

SEQ ID NO: 820 - protein, 5 aa, synthetic construct
  REGION 1..5: note = Synthetic peptide
GSTEN

SEQ ID NO: 821 - protein, 4 aa, synthetic construct
  REGION 1..4: note = Synthetic peptide
STEN

SEQ ID NO: 822 - protein, 10 aa, synthetic construct
  REGION 1..10: note = Synthetic peptide
KSKIGSTENL

SEQ ID NO: 823 - protein, 9 aa, synthetic construct
  REGION 1..9: note = Synthetic peptide
SKIGSTENL

SEQ ID NO: 824 - protein, 8 aa, synthetic construct
  REGION 1..8: note = Synthetic peptide
KIGSTENL

SEQ ID NO: 825 - protein, 7 aa, synthetic construct
  REGION 1..7: note = Synthetic peptide
IGSTENL

SEQ ID NO: 826 - protein, 6 aa, synthetic construct
  REGION 1..6: note = Synthetic peptide
GSTENL

SEQ ID NO: 827 - protein, 5 aa, synthetic construct
  REGION 1..5: note = Synthetic peptide
STENL

SEQ ID NO: 828 - protein, 10 aa, synthetic construct
  REGION 1..10: note = Synthetic peptide
SKIGSTENLK

SEQ ID NO: 829 - protein, 9 aa, synthetic construct
  REGION 1..9: note = Synthetic peptide
KIGSTENLK

SEQ ID NO: 830 - protein, 8 aa, synthetic construct
  REGION 1..8: note = Synthetic peptide
IGSTENLK

SEQ ID NO: 831 - protein, 7 aa, synthetic construct
  REGION 1..7: note = Synthetic peptide
GSTENLK

SEQ ID NO: 832 - protein, 6 aa, synthetic construct
  REGION 1..6: note = Synthetic peptide
STENLK

SEQ ID NO: 833 - protein, 10 aa, synthetic construct
  REGION 1..10: note = Synthetic peptide
KIGSTENLKH

SEQ ID NO: 834 - protein, 9 aa, synthetic construct
  REGION 1..9: note = Synthetic peptide
IGSTENLKH

SEQ ID NO: 835 - protein, 8 aa, synthetic construct
  REGION 1..8: note = Synthetic peptide
GSTENLKH

SEQ ID NO: 836 - protein, 7 aa, synthetic construct
  REGION 1..7: note = Synthetic peptide
STENLKH

SEQ ID NO: 837 - protein, 10 aa, synthetic construct
  REGION 1..10: note = Synthetic peptide
IGSTENLKHQ

SEQ ID NO: 838 - protein, 9 aa, synthetic construct
  REGION 1..9: note = Synthetic peptide
GSTENLKHQ

SEQ ID NO: 839 - protein, 8 aa, synthetic construct
  REGION 1..8: note = Synthetic peptide
STENLKHQ

SEQ ID NO: 840 - protein, 10 aa, synthetic construct
  REGION 1..10: note = Synthetic peptide
GSTENLKHQP

SEQ ID NO: 841 - protein, 9 aa, synthetic construct
  REGION 1..9: note = Synthetic peptide
STENLKHQP

SEQ ID NO: 842 - protein, 10 aa, synthetic construct
  REGION 1..10: note = Synthetic peptide
STENLKHQPG

SEQ ID NO: 843 - protein, 10 aa, synthetic construct
  REGION 1..10: note = Synthetic peptide
SNVQSKCGSK

SEQ ID NO: 844 - protein, 9 aa, synthetic construct
  REGION 1..9: note = Synthetic peptide
NVQSKCGSK

SEQ ID NO: 845 - protein, 8 aa, synthetic construct
  REGION 1..8: note = Synthetic peptide
VQSKCGSK

SEQ ID NO: 846 - protein, 7 aa, synthetic construct
  REGION 1..7: note = Synthetic peptide
QSKCGSK

SEQ ID NO: 847 - protein, 6 aa, synthetic construct
  REGION 1..6: note = Synthetic peptide
SKCGSK

SEQ ID NO: 848 - protein, 5 aa, synthetic construct
  REGION 1..5: note = Synthetic peptide
KCGSK

SEQ ID NO: 849 - protein, 4 aa, synthetic construct
  REGION 1..4: note = Synthetic peptide
CGSK

SEQ ID NO: 850 - skipped

SEQ ID NO: 851 - protein, 10 aa, synthetic construct
  REGION 1..10: note = Synthetic peptide
NVQSKCGSKD

SEQ ID NO: 852 - protein, 9 aa, synthetic construct
  REGION 1..9: note = Synthetic peptide
VQSKCGSKD

SEQ ID NO: 853 - protein, 8 aa, synthetic construct
  REGION 1..8: note = Synthetic peptide
QSKCGSKD

SEQ ID NO: 854 - protein, 7 aa, synthetic construct
  REGION 1..7: note = Synthetic peptide
SKCGSKD

SEQ ID NO: 855 - protein, 6 aa, synthetic construct
  REGION 1..6: note = Synthetic peptide
KCGSKD

SEQ ID NO: 856 - protein, 5 aa, synthetic construct
  REGION 1..5: note = Synthetic peptide
CGSKD

SEQ ID NO: 857 - protein, 4 aa, synthetic construct
  REGION 1..4: note = Synthetic peptide
GSKD

SEQ ID NO: 858 - skipped

SEQ ID NO: 859 - protein, 10 aa, synthetic construct
  REGION 1..10: note = Synthetic peptide
VQSKCGSKDN

SEQ ID NO: 860 - protein, 9 aa, synthetic construct
  REGION 1..9: note = Synthetic peptide
QSKCGSKDN

SEQ ID NO: 861 - protein, 8 aa, synthetic construct
  REGION 1..8: note = Synthetic peptide
SKCGSKDN

SEQ ID NO: 862 - protein, 7 aa, synthetic construct
  REGION 1..7: note = Synthetic peptide
KCGSKDN

SEQ ID NO: 863 - protein, 6 aa, synthetic construct
  REGION 1..6: note = Synthetic peptide
CGSKDN

SEQ ID NO: 864 - protein, 5 aa, synthetic construct
  REGION 1..5: note = Synthetic peptide
GSKDN

SEQ ID NO: 865 - protein, 4 aa, synthetic construct
  REGION 1..4: note = Synthetic peptide
SKDN

SEQ ID NO: 866 - protein, 10 aa, synthetic construct
  REGION 1..10: note = Synthetic peptide
QSKCGSKDNI

SEQ ID NO: 867 - protein, 9 aa, synthetic construct
  REGION 1..9: note = Synthetic peptide
SKCGSKDNI

SEQ ID NO: 868 - protein, 8 aa, synthetic construct
  REGION 1..8: note = Synthetic peptide
KCGSKDNI

SEQ ID NO: 869 - protein, 7 aa, synthetic construct
  REGION 1..7: note = Synthetic peptide
CGSKDNI

SEQ ID NO: 870 - protein, 6 aa, synthetic construct
  REGION 1..6: note = Synthetic peptide
GSKDNI

SEQ ID NO: 871 - protein, 5 aa, synthetic construct
  REGION 1..5: note = Synthetic peptide
SKDNI

SEQ ID NO: 872 - protein, 10 aa, synthetic construct
  REGION 1..10: note = Synthetic peptide
SKVTSKCGSL

SEQ ID NO: 873 - protein, 9 aa, synthetic construct
  REGION 1..9: note = Synthetic peptide
KVTSKCGSL

SEQ ID NO: 874 - protein, 8 aa, synthetic construct
  REGION 1..8: note = Synthetic peptide
VTSKCGSL

SEQ ID NO: 875 - protein, 7 aa, synthetic construct
  REGION 1..7: note = Synthetic peptide
TSKCGSL

SEQ ID NO: 876 - protein, 6 aa, synthetic construct
  REGION 1..6: note = Synthetic peptide
SKCGSL

SEQ ID NO: 877 - protein, 5 aa, synthetic construct
  REGION 1..5: note = Synthetic peptide
KCGSL

SEQ ID NO: 878 - protein, 4 aa, synthetic construct
  REGION 1..4: note = Synthetic peptide
CGSL

SEQ ID NO: 879 - skipped

SEQ ID NO: 880 - protein, 10 aa, synthetic construct
  REGION 1..10: note = Synthetic peptide
KVTSKCGSLG

SEQ ID NO: 881 - protein, 9 aa, synthetic construct
  REGION 1..9: note = Synthetic peptide
VTSKCGSLG

SEQ ID NO: 882 - protein, 8 aa, synthetic construct
  REGION 1..8: note = Synthetic peptide
TSKCGSLG

SEQ ID NO: 883 - protein, 7 aa, synthetic construct
  REGION 1..7: note = Synthetic peptide
SKCGSLG

SEQ ID NO: 884 - protein, 6 aa, synthetic construct
  REGION 1..6: note = Synthetic peptide
KCGSLG

SEQ ID NO: 885 - protein, 5 aa, synthetic construct
  REGION 1..5: note = Synthetic peptide
CGSLG

SEQ ID NO: 886 - protein, 4 aa, synthetic construct
  REGION 1..4: note = Synthetic peptide
GSLG

SEQ ID NO: 887 - skipped

SEQ ID NO: 888 - protein, 10 aa, synthetic construct
  REGION 1..10: note = Synthetic peptide
VTSKCGSLGN

SEQ ID NO: 889 - protein, 9 aa, synthetic construct
  REGION 1..9: note = Synthetic peptide
TSKCGSLGN

SEQ ID NO: 890 - protein, 8 aa, synthetic construct
  REGION 1..8: note = Synthetic peptide
SKCGSLGN

SEQ ID NO: 891 - protein, 7 aa, synthetic construct
  REGION 1..7: note = Synthetic peptide
KCGSLGN

SEQ ID NO: 892 - protein, 6 aa, synthetic construct
  REGION 1..6: note = Synthetic peptide
CGSLGN

SEQ ID NO: 893 - protein, 5 aa, synthetic construct
  REGION 1..5: note = Synthetic peptide
GSLGN

SEQ ID NO: 894 - protein, 4 aa, synthetic construct
  REGION 1..4: note = Synthetic peptide
SLGN

SEQ ID NO: 895 - protein, 10 aa, synthetic construct
  REGION 1..10: note = Synthetic peptide
TSKCGSLGNI

SEQ ID NO: 896 - protein, 9 aa, synthetic construct
  REGION 1..9: note = Synthetic peptide
SKCGSLGNI

SEQ ID NO: 897 - protein, 8 aa, synthetic construct
  REGION 1..8: note = Synthetic peptide
KCGSLGNI

SEQ ID NO: 898 - protein, 7 aa, synthetic construct
  REGION 1..7: note = Synthetic peptide
CGSLGNI

SEQ ID NO: 899 - protein, 6 aa, synthetic construct
  REGION 1..6: note = Synthetic peptide
GSLGNI

SEQ ID NO: 900 - protein, 5 aa, synthetic construct
  REGION 1..5: note = Synthetic peptide
SLGNI

SEQ ID NO: 901 - protein, 10 aa, synthetic construct
  REGION 1..10: note = Synthetic peptide
SKCGSLGNIH

SEQ ID NO: 902 - protein, 9 aa, synthetic construct
  REGION 1..9: note = Synthetic peptide
KCGSLGNIH

SEQ ID NO: 903 - protein, 8 aa, synthetic construct
  REGION 1..8: note = Synthetic peptide
CGSLGNIH

SEQ ID NO: 904 - protein, 7 aa, synthetic construct
  REGION 1..7: note = Synthetic peptide
GSLGNIH

SEQ ID NO: 905 - protein, 6 aa, synthetic construct
  REGION 1..6: note = Synthetic peptide
SLGNIH

SEQ ID NO: 906 - protein, 10 aa, synthetic construct
  REGION 1..10: note = Synthetic peptide
KCGSLGNIHH

SEQ ID NO: 907 - protein, 9 aa, synthetic construct
  REGION 1..9: note = Synthetic peptide
CGSLGNIHH

SEQ ID NO: 908 - protein, 8 aa, synthetic construct
  REGION 1..8: note = Synthetic peptide
GSLGNIHH

SEQ ID NO: 909 - protein, 7 aa, synthetic construct
  REGION 1..7: note = Synthetic peptide
SLGNIHH

SEQ ID NO: 910 - protein, 10 aa, synthetic construct
  REGION 1..10: note = Synthetic peptide
CGSLGNIHHK

SEQ ID NO: 911 - protein, 9 aa, synthetic construct
  REGION 1..9: note = Synthetic peptide
GSLGNIHHK

SEQ ID NO: 912 - protein, 8 aa, synthetic construct
  REGION 1..8: note = Synthetic peptide
SLGNIHHK

SEQ ID NO: 913 - protein, 10 aa, synthetic construct
  REGION 1..10: note = Synthetic peptide
GSLGNIHHKP

SEQ ID NO: 914 - protein, 9 aa, synthetic construct
  REGION 1..9: note = Synthetic peptide
SLGNIHHKP

SEQ ID NO: 915 - protein, 10 aa, synthetic construct
  REGION 1..10: note = Synthetic peptide
SLGNIHHKPG

SEQ ID NO: 916 - protein, 10 aa, synthetic construct
  REGION 1..10: note = Synthetic peptide
DRVQSKIGSL

SEQ ID NO: 917 - protein, 9 aa, synthetic construct
  REGION 1..9: note = Synthetic peptide
RVQSKIGSL

SEQ ID NO: 918 - protein, 8 aa, synthetic construct
  REGION 1..8: note = Synthetic peptide
VQSKIGSL

SEQ ID NO: 919 - protein, 7 aa, synthetic construct
  REGION 1..7: note = Synthetic peptide
QSKIGSL

SEQ ID NO: 920 - protein, 6 aa, synthetic construct
  REGION 1..6: note = Synthetic peptide
SKIGSL

SEQ ID NO: 921 - protein, 5 aa, synthetic construct
  REGION 1..5: note = Synthetic peptide
KIGSL

SEQ ID NO: 922 - protein, 4 aa, synthetic construct
  REGION 1..4: note = Synthetic peptide
IGSL

SEQ ID NO: 923 - protein, 10 aa, synthetic construct
  REGION 1..10: note = Synthetic peptide
RVQSKIGSLD

SEQ ID NO: 924 - protein, 9 aa, synthetic construct
  REGION 1..9: note = Synthetic peptide
VQSKIGSLD

SEQ ID NO: 925 - protein, 8 aa, synthetic construct
  REGION 1..8: note = Synthetic peptide
QSKIGSLD

SEQ ID NO: 926 - protein, 7 aa, synthetic construct
  REGION 1..7: note = Synthetic peptide
SKIGSLD

SEQ ID NO: 927 - protein, 6 aa, synthetic construct
  REGION 1..6: note = Synthetic peptide
KIGSLD

SEQ ID NO: 928 - protein, 5 aa, synthetic construct
  REGION 1..5: note = Synthetic peptide
IGSLD

SEQ ID NO: 929 - protein, 4 aa, synthetic construct
  REGION 1..4: note = Synthetic peptide
GSLD

SEQ ID NO: 930 - skipped

SEQ ID NO: 931 - protein, 10 aa, synthetic construct
  REGION 1..10: note = Synthetic peptide
VQSKIGSLDN

SEQ ID NO: 932 - protein, 9 aa, synthetic construct
  REGION 1..9: note = Synthetic peptide
QSKIGSLDN

SEQ ID NO: 933 - protein, 8 aa, synthetic construct
  REGION 1..8: note = Synthetic peptide
SKIGSLDN

SEQ ID NO: 934 - protein, 7 aa, synthetic construct
  REGION 1..7: note = Synthetic peptide
KIGSLDN

SEQ ID NO: 935 - protein, 6 aa, synthetic construct
  REGION 1..6: note = Synthetic peptide
IGSLDN

SEQ ID NO: 936 - protein, 5 aa, synthetic construct
  REGION 1..5: note = Synthetic peptide
GSLDN

SEQ ID NO: 937 - protein, 4 aa, synthetic construct
  REGION 1..4: note = Synthetic peptide
SLDN

SEQ ID NO: 938 - protein, 10 aa, synthetic construct
  REGION 1..10: note = Synthetic peptide
QSKIGSLDNI

SEQ ID NO: 939 - protein, 9 aa, synthetic construct
  REGION 1..9: note = Synthetic peptide
SKIGSLDNI

SEQ ID NO: 940 - protein, 8 aa, synthetic construct
  REGION 1..8: note = Synthetic peptide
KIGSLDNI

SEQ ID NO: 941 - protein, 7 aa, synthetic construct
  REGION 1..7: note = Synthetic peptide
IGSLDNI

SEQ ID NO: 942 - protein, 6 aa, synthetic construct
  REGION 1..6: note = Synthetic peptide
GSLDNI

SEQ ID NO: 943 - protein, 10 aa, synthetic construct
  REGION 1..10: note = Synthetic peptide
SKIGSLDNIT

SEQ ID NO: 944 - protein, 9 aa, synthetic construct
  REGION 1..9: note = Synthetic peptide
KIGSLDNIT

SEQ ID NO: 945 - protein, 8 aa, synthetic construct
  REGION 1..8: note = Synthetic peptide
IGSLDNIT

SEQ ID NO: 946 - protein, 7 aa, synthetic construct
  REGION 1..7: note = Synthetic peptide
GSLDNIT

SEQ ID NO: 947 - protein, 6 aa, synthetic construct
  REGION 1..6: note = Synthetic peptide
SLDNIT

SEQ ID NO: 948 - protein, 10 aa, synthetic construct
  REGION 1..10: note = Synthetic peptide
KIGSLDNITH

SEQ ID NO: 949 - protein, 9 aa, synthetic construct
  REGION 1..9: note = Synthetic peptide
IGSLDNITH

SEQ ID NO: 950 - protein, 8 aa, synthetic construct
  REGION 1..8: note = Synthetic peptide
GSLDNITH

SEQ ID NO: 951 - protein, 7 aa, synthetic construct
  REGION 1..7: note = Synthetic peptide
SLDNITH

SEQ ID NO: 952 - protein, 10 aa, synthetic construct
  REGION 1..10: note = Synthetic peptide
IGSLDNITHV

SEQ ID NO: 953 - protein, 9 aa, synthetic construct
  REGION 1..9: note = Synthetic peptide
GSLDNITHV

SEQ ID NO: 954 - protein, 8 aa, synthetic construct
  REGION 1..8: note = Synthetic peptide
SLDNITHV

SEQ ID NO: 955 - protein, 10 aa, synthetic construct
  REGION 1..10: note = Synthetic peptide
GSLDNITHVP

SEQ ID NO: 956 - protein, 9 aa, synthetic construct
  REGION 1..9: note = Synthetic peptide
SLDNITHVP

SEQ ID NO: 957 - protein, 10 aa, synthetic construct
  REGION 1..10: note = Synthetic peptide
SLDNITHVPG

SEQ ID NO: 958 - protein, 8 aa, synthetic construct
  REGION 1..8: note = Synthetic peptide
PDLKNVKS

SEQ ID NO: 959 - protein, 8 aa, synthetic construct
  REGION 1..8: note = Synthetic peptide
DLKNVKSK

SEQ ID NO: 960 - protein, 8 aa, synthetic construct
  REGION 1..8: note = Synthetic peptide
LKNVKSKI

SEQ ID NO: 961 - protein, 8 aa, synthetic construct
  REGION 1..8: note = Synthetic peptide
KNVKSKIG

SEQ ID NO: 962 - protein, 8 aa, synthetic construct
  REGION 1..8: note = Synthetic peptide
NVKSKIGS

SEQ ID NO: 963 - protein, 8 aa, synthetic construct
  REGION 1..8: note = Synthetic peptide
DLSNVQSK

SEQ ID NO: 964 - protein, 8 aa, synthetic construct
  REGION 1..8: note = Synthetic peptide
LSNVQSKC

SEQ ID NO: 965 - protein, 8 aa, synthetic construct
  REGION 1..8: note = Synthetic peptide
SNVQSKCG

SEQ ID NO: 966 - protein, 8 aa, synthetic construct
  REGION 1..8: note = Synthetic peptide
NVQSKCGS

SEQ ID NO: 967 - protein, 8 aa, synthetic construct
  REGION 1..8: note = Synthetic peptide
VDLSKVTS

SEQ ID NO: 968 - protein, 8 aa, synthetic construct
  REGION 1..8: note = Synthetic peptide
DLSKVTSK

SEQ ID NO: 969 - protein, 8 aa, synthetic construct
  REGION 1..8: note = Synthetic peptide
LSKVTSKC

SEQ ID NO: 970 - protein, 8 aa, synthetic construct
  REGION 1..8: note = Synthetic peptide
SKVTSKCG

SEQ ID NO: 971 - protein, 8 aa, synthetic construct
  REGION 1..8: note = Synthetic peptide
KVTSKCGS

SEQ ID NO: 972 - protein, 8 aa, synthetic construct
  REGION 1..8: note = Synthetic peptide
LDFKDRVQ

SEQ ID NO: 973 - protein, 8 aa, synthetic construct
  REGION 1..8: note = Synthetic peptide
DFKDRVQS

SEQ ID NO: 974 - protein, 8 aa, synthetic construct
  REGION 1..8: note = Synthetic peptide
FKDRVQSK

SEQ ID NO: 975 - protein, 8 aa, synthetic construct
  REGION 1..8: note = Synthetic peptide
KDRVQSKI

SEQ ID NO: 976 - protein, 8 aa, synthetic construct
  REGION 1..8: note = Synthetic peptide
DRVQSKIG

SEQ ID NO: 977 - protein, 8 aa, synthetic construct
  REGION 1..8: note = Synthetic peptide
RVQSKIGS

SEQ ID NO: 978 - protein, 11 aa, synthetic construct
  REGION 1..11: note = Synthetic peptide
SKIGSTENLKH

SEQ ID NO: 979 - protein, 11 aa, synthetic construct
  REGION 1..11: note = Synthetic peptide
SKIGSTENIKH

SEQ ID NO: 980 - protein, 11 aa, synthetic construct
  REGION 1..11: note = Synthetic peptide
SKIGSKDNLKH

SEQ ID NO: 981 - protein, 11 aa, synthetic construct
  REGION 1..11: note = Synthetic peptide
SKIGSKENIKH

SEQ ID NO: 982 - protein, 11 aa, synthetic construct
  REGION 1..11: note = Synthetic peptide
SKIGSLENLKH

SEQ ID NO: 983 - protein, 11 aa, synthetic construct
  REGION 1..11: note = Synthetic peptide
SKIGSLENIKH

SEQ ID NO: 984 - protein, 11 aa, synthetic construct
  REGION 1..11: note = Synthetic peptide
SKIGSTDNLKH

SEQ ID NO: 985 - protein, 11 aa, synthetic construct
  REGION 1..11: note = Synthetic peptide
SKIGSTDNIKH

SEQ ID NO: 986 - protein, 11 aa, synthetic construct
  REGION 1..11: note = Synthetic peptide
SKIGSKDNIKH

SEQ ID NO: 987 - protein, 11 aa, synthetic construct
  REGION 1..11: note = Synthetic peptide
SKIGSLDNLKH

SEQ ID NO: 988 - protein, 11 aa, synthetic construct
  REGION 1..11: note = Synthetic peptide
SKIGSLDNIKH

SEQ ID NO: 989 - protein, 11 aa, synthetic construct
  REGION 1..11: note = Synthetic peptide
SKIGSTGNLKH

SEQ ID NO: 990 - protein, 11 aa, synthetic construct
  REGION 1..11: note = Synthetic peptide
SKIGSTGNIKH

SEQ ID NO: 991 - protein, 11 aa, synthetic construct
  REGION 1..11: note = Synthetic peptide
SKIGSKGNLKH

SEQ ID NO: 992 - protein, 11 aa, synthetic construct
  REGION 1..11: note = Synthetic peptide
SKIGSKGNIKH

SEQ ID NO: 993 - protein, 11 aa, synthetic construct
  REGION 1..11: note = Synthetic peptide
SKIGSLGNLKH

SEQ ID NO: 994 - protein, 11 aa, synthetic construct
  REGION 1..11: note = Synthetic peptide
SKIGSLGNIKH

SEQ ID NO: 995 - protein, 10 aa, synthetic construct
  REGION 1..10: note = Synthetic peptide
  VAR_SEQ 2: note = MISC_FEATURE - Xaa is I or C
  VAR_SEQ 3: note = MISC_FEATURE - Xaa is G
  VAR_SEQ 5: note = MISC_FEATURE - Xaa is T or K or L
  VAR_SEQ 6: note = MISC_FEATURE - Xaa is E or D or G
  VAR_SEQ 8: note = MISC_FEATURE - Xaa is L or I
  VAR_SEQ 9: note = MISC_FEATURE - Xaa is K or H or T
KXXSXXNXXH

SEQ ID NO: 996 - protein, 6 aa, synthetic construct
  REGION 1..6: note = Synthetic peptide
  VAR_SEQ 1: note = MISC_FEATURE - Xaa can be Q or E
  VAR_SEQ 6: note = MISC_FEATURE - Xaa can be S or P
XIVYKX

SEQ ID NO: 997 - protein, 20 aa, synthetic construct
  REGION 1..20: note = Synthetic peptide
DAEFRHDRRVKSKIGSTGGC

SEQ ID NO: 998 - protein, 20 aa, synthetic construct
  REGION 1..20: note = Synthetic peptide
DAEFRHDRRSKIGSTENGGC

SEQ ID NO: 999 - protein, 20 aa, synthetic construct
  REGION 1..20: note = Synthetic peptide
DAEFRHDRRTENLKHQPGGC

SEQ ID NO: 1000 - protein, 20 aa, synthetic construct
  REGION 1..20: note = Synthetic peptide
DAEFRHDRRENLKHQPGGGC

SEQ ID NO: 1001 - protein, 23 aa, synthetic construct
  REGION 1..23: note = Synthetic peptide
DAEFRHDRRSKIGSKDNIKHGGC

SEQ ID NO: 1002 - protein, 10 aa, synthetic construct
  REGION 1..10: note = Synthetic peptide
VHHQKLVFFA

SEQ ID NO: 1003 - protein, 9 aa, synthetic construct
  REGION 1..9: note = Synthetic peptide
VHHQKLVFF

SEQ ID NO: 1004 - protein, 8 aa, synthetic construct
  REGION 1..8: note = Synthetic peptide
VHHQKLVF

SEQ ID NO: 1005 - protein, 7 aa, synthetic construct
  REGION 1..7: note = Synthetic peptide
VHHQKLV

SEQ ID NO: 1006 - protein, 6 aa, synthetic construct
  REGION 1..6: note = Synthetic peptide
VHHQKL

SEQ ID NO: 1007 - protein, 10 aa, synthetic construct
  REGION 1..10: note = Synthetic peptide
HHQKLVFFAE

SEQ ID NO: 1008 - protein, 9 aa, synthetic construct
  REGION 1..9: note = Synthetic peptide
HHQKLVFFA

SEQ ID NO: 1009 - protein, 8 aa, synthetic construct
  REGION 1..8: note = Synthetic peptide
HHQKLVFF

SEQ ID NO: 1010 - protein, 7 aa, synthetic construct
  REGION 1..7: note = Synthetic peptide
HHQKLVF

SEQ ID NO: 1011 - protein, 6 aa, synthetic construct
  REGION 1..6: note = Synthetic peptide
HHQKLV

SEQ ID NO: 1012 - protein, 5 aa, synthetic construct
  REGION 1..5: note = Synthetic peptide
HHQKL

SEQ ID NO: 1013 - protein, 10 aa, synthetic construct
  REGION 1..10: note = Synthetic peptide
HQKLVFFAED

SEQ ID NO: 1014 - protein, 9 aa, synthetic construct
  REGION 1..9: note = Synthetic peptide
HQKLVFFAE

SEQ ID NO: 1015 - protein, 8 aa, synthetic construct
  REGION 1..8: note = Synthetic peptide
HQKLVFFA

SEQ ID NO: 1016 - protein, 7 aa, synthetic construct
  REGION 1..7: note = Synthetic peptide
HQKLVFF

SEQ ID NO: 1017 - protein, 6 aa, synthetic construct
  REGION 1..6: note = Synthetic peptide
HQKLVF

SEQ ID NO: 1018 - protein, 5 aa, synthetic construct
  REGION 1..5: note = Synthetic peptide
HQKLV

SEQ ID NO: 1019 - protein, 4 aa, synthetic construct
  REGION 1..4: note = Synthetic peptide
HQKL

SEQ ID NO: 1020 - protein, 10 aa, synthetic construct
  REGION 1..10: note = Synthetic peptide
QKLVFFAEDV

SEQ ID NO: 1021 - protein, 9 aa, synthetic construct
  REGION 1..9: note = Synthetic peptide
QKLVFFAED

SEQ ID NO: 1022 - protein, 8 aa, synthetic construct
  REGION 1..8: note = Synthetic peptide
QKLVFFAE

SEQ ID NO: 1023 - protein, 7 aa, synthetic construct
  REGION 1..7: note = Synthetic peptide
QKLVFFA

SEQ ID NO: 1024 - protein, 6 aa, synthetic construct
  REGION 1..6: note = Synthetic peptide
QKLVFF

SEQ ID NO: 1025 - protein, 5 aa, synthetic construct
  REGION 1..5: note = Synthetic peptide
QKLVF

SEQ ID NO: 1026 - protein, 4 aa, synthetic construct
  REGION 1..4: note = Synthetic peptide
QKLV

SEQ ID NO: 1027 - skipped

SEQ ID NO: 1028 - protein, 10 aa, synthetic construct
  REGION 1..10: note = Synthetic peptide
KLVFFAEDVG

SEQ ID NO: 1029 - protein, 9 aa, synthetic construct
  REGION 1..9: note = Synthetic peptide
KLVFFAEDV

SEQ ID NO: 1030 - protein, 8 aa, synthetic construct
  REGION 1..8: note = Synthetic peptide
KLVFFAED

SEQ ID NO: 1031 - protein, 7 aa, synthetic construct
  REGION 1..7: note = Synthetic peptide
KLVFFAE

SEQ ID NO: 1032 - protein, 6 aa, synthetic construct
  REGION 1..6: note = Synthetic peptide
KLVFFA

SEQ ID NO: 1033 - protein, 5 aa, synthetic construct
  REGION 1..5: note = Synthetic peptide
KLVFF

SEQ ID NO: 1034 - protein, 4 aa, synthetic construct
  REGION 1..4: note = Synthetic peptide
KLVF

SEQ ID NO: 1035 - protein, 4 aa, synthetic construct
  REGION 1..4: note = Synthetic peptide
KLVF

SEQ ID NO: 1036 - protein, 9 aa, synthetic construct
  REGION 1..9: note = Synthetic peptide
LVFFAEDVG

SEQ ID NO: 1037 - protein, 8 aa, synthetic construct
  REGION 1..8: note = Synthetic peptide
LVFFAEDV

SEQ ID NO: 1038 - protein, 7 aa, synthetic construct
  REGION 1..7: note = Synthetic peptide
LVFFAED

SEQ ID NO: 1039 - protein, 6 aa, synthetic construct
  REGION 1..6: note = Synthetic peptide
LVFFAE

SEQ ID NO: 1040 - protein, 5 aa, synthetic construct
  REGION 1..5: note = Synthetic peptide
LVFFA

SEQ ID NO: 1041 - protein, 4 aa, synthetic construct
  REGION 1..4: note = Synthetic peptide
LVFF

SEQ ID NO: 1042 - skipped

SEQ ID NO: 1043 - protein, 8 aa, synthetic construct
  REGION 1..8: note = Synthetic peptide
VFFAEDVG

SEQ ID NO: 1044 - protein, 7 aa, synthetic construct
  REGION 1..7: note = Synthetic peptide
VFFAEDV

SEQ ID NO: 1045 - protein, 6 aa, synthetic construct
  REGION 1..6: note = Synthetic peptide
VFFAED

SEQ ID NO: 1046 - protein, 5 aa, synthetic construct
  REGION 1..5: note = Synthetic peptide
VFFAE

SEQ ID NO: 1047 - protein, 4 aa, synthetic construct
  REGION 1..4: note = Synthetic peptide
VFFA

SEQ ID NO: 1048 - skipped

SEQ ID NO: 1049 - protein, 7 aa, synthetic construct
  REGION 1..7: note = Synthetic peptide
FFAEDVG

SEQ ID NO: 1050 - protein, 6 aa, synthetic construct
  REGION 1..6: note = Synthetic peptide
FFAEDV

SEQ ID NO: 1051 - protein, 5 aa, synthetic construct
  REGION 1..5: note = Synthetic peptide
FFAED

SEQ ID NO: 1052 - protein, 4 aa, synthetic construct
  REGION 1..4: note = Synthetic peptide
FFAE

SEQ ID NO: 1053 - skipped

SEQ ID NO: 1054 - protein, 6 aa, synthetic construct
  REGION 1..6: note = Synthetic peptide
FAEDVG

SEQ ID NO: 1055 - protein, 5 aa, synthetic construct
  REGION 1..5: note = Synthetic peptide
FAEDV

SEQ ID NO: 1056 - protein, 4 aa, synthetic construct
  REGION 1..4: note = Synthetic peptide
FAED

SEQ ID NO: 1057 - skipped

SEQ ID NO: 1058 - protein, 31 aa, synthetic construct
  REGION 1..31: note = Synthetic peptide
QTAPVPMPDLKNVKSKIGSTENLKHQPGGGK

SEQ ID NO: 1059 - protein, 31 aa, synthetic construct
  REGION 1..31: note = Synthetic peptide
VQIINKKLDLSNVQSKCGSKDNIKHVPGGGS

SEQ ID NO: 1060 - protein, 31 aa, synthetic construct
  REGION 1..31: note = Synthetic peptide
VQIVYKPVDLSKVTSKCGSLGNIHHKPGGGQ

SEQ ID NO: 1061 - protein, 32 aa, synthetic construct
  REGION 1..32: note = Synthetic peptide
VEVKSEKLDFKDRVQSKIGSLDNITHVPGGGN

SEQ ID NO: 1062 - protein, 4 aa, synthetic construct
  REGION 1..4: note = linker
GGGS

SEQ ID NO: 1063 - protein, 5 aa, synthetic construct
  REGION 1..5: note = linker
GGGGS